SEQUENCE LISTING (23 sequences)

SEQ ID NO: 1 - protein, 894 aa, Homo Sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 2 - protein, 893 aa, Macaca Fascicularis
AWRCPRMGRVPLAWCLALCGWVCMAPRGTQAEESPFVGNPGNITGARGLTGTLRCQLQVQGEPPEVHWLRDGQILELADSTQTQVPLGEDEQDDWIVVSQLRIASLQLSDAGQYQCLVFLGHQNFVSQPGYVGLEGLPYFLEEPEDRTVAANTPFNLSCQAQGPPEPVDLLWLQDAVPLATAPGHGPQRNLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTLQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKETSAPAFSWPWWYILLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQLDPKDSCSCLTSAEVHPAGRYVLCPSTAPSPAQPADRGSPAAPGQEDGA

SEQ ID NO: 3 - protein, 1209 aa, Homo Sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 4 - protein, 1620 aa, Homo Sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 5 - protein, 981 aa, Homo Sapiens
MDGFAGSLDDSISAASTSDVQDRLSALESRVQQQEDEITVLKAALADVLRRLAISEDHVASVKKSVSSKGQPSPRAVIPMSCITNGSGANRKPSHTSAVSIAGKETLSSAAKSGTEKKKEKPQGQREKKEESHSNDQSPQIRASPSPQPSSQPLQIHRQTPESKNATPTKSIKRPSPAEKSHNSWENSDDSRNKLSKIPSTPKLIPKVTKTADKHKDVIINQEGEYIKMFMRGRPITMFIPSDVDNYDDIRTELPPEKLKLEWAYGYRGKDCRANVYLLPTGKIVYFIASVVVLFNYEERTQRHYLGHTDCVKCLAIHPDKIRIATGQIAGVDKDGRPLQPHVRVWDSVTLSTLQIIGLGTFERGVGCLDFSKADSGVHLCIIDDSNEHMLTVWDWQKKAKGAEIKTTNEVVLAVEFHPTDANTIITCGKSHIFFWTWSGNSLTRKQGIFGKYEKPKFVQCLAFLGNGDVLTGDSGGVMLIWSKTTVEPTPGKGPKGVYQISKQIKAHDGSVFTLCQMRNGMLLTGGGKDRKIILWDHDLNPEREIEVPDQYGTIRAVAEGKADQFLVGTSRNFILRGTFNDGFQIEVQGHTDELWGLATHPFKDLLLTCAQDRQVCLWNSMEHRLEWTRLVDEPGHCADFHPSGTVVAIGTHSGRWFVLDAETRDLVSIHTDGNEQLSVMRYSIDGTFLAVGSHDNFIYLYVVSENGRKYSRYGRCTGHSSYITHLDWSPDNKYIMSNSGDYEILYWDIPNGCKLIRNRSDCKDIDWTTYTCVLGFQVFGVWPEGSDGTDINALVRSHNRKVIAVADDFCKVHLFQYPCSKAKAPSHKYSAHSSHVTNVSFTHNDSHLISTGGKDMSIIQWKLVEKLSLPQNETVADTTLTKAPVSSTESVIQSNTPTPPPSQPLNETAEEESRISSSPTLLENSLEQTVEPSEDHSEEESEEGSGDLGEPLYEEPCNEISKEQAKATLLEDQQDPSPSS

SEQ ID NO: 6 - protein, 963 aa, Homo Sapiens
MADLAECNIKVMCRFRPLNESEVNRGDKYIAKFQGEDTVVIASKPYAFDRVFQSSTSQEQVYNDCAKKIVKDVLEGYNGTIFAYGQTSSGKTHTMEGKLHDPEGMGIIPRIVQDIFNYIYSMDENLEFHIKVSYFEIYLDKIRDLLDVSKTNLSVHEDKNRVPYVKGCTERFVCSPDEVMDTIDEGKSNRHVAVTNMNEHSSRSHSIFLINVKQENTQTEQKLSGKLYLVDLAGSEKVSKTGAEGAVLDEAKNINKSLSALGNVISALAEGSTYVPYRDSKMTRILQDSLGGNCRTTIVICCSPSSYNESETKSTLLFGQRAKTIKNTVCVNVELTAEQWKKKYEKEKEKNKILRNTIQWLENELNRWRNGETVPIDEQFDKEKANLEAFTVDKDITLTNDKPATAIGVIGNFTDAERRKCEEEIAKLYKQLDDKDEEINQQSQLVEKLKTQMLDQEELLASTRRDQDNMQAELNRLQAENDASKEEVKEVLQALEELAVNYDQKSQEVEDKTKEYELLSDELNQKSATLASIDAELQKLKEMTNHQKKRAAEMMASLLKDLAEIGIAVGNNDVKQPEGTGMIDEEFTVARLYISKMKSEVKTMVKRCKQLESTQTESNKKMEENEKELAACQLRISQHEAKIKSLTEYLQNVEQKKRQLEESVDALSEELVQLRAQEKVHEMEKEHLNKVQTANEVKQAVEQQIQSHRETHQKQISSLRDEVEAKAKLITDLQDQNQKMMLEQERLRVEHEKLKATDQEKSRKLHELTVMQDRREQARQDLKGLEETVAKELQTLHNLRKLFVQDLATRVKKSAEIDSDDTGGSAAQKQKISFLENNLEQLTKVHKQLVRDNADLRCELPKLEKRLRATAERVKALESALKEAKENASRDRKRYQQEVDRIKEAVRSKNMARRGHSAQIAKPIRPGQHPAASPTHPSAIRGGGAFVQNSQPVAVRGGGGKQV

SEQ ID NO: 7 - protein, 573 aa, Homo Sapiens
MYDNMSTMVYIKEDKLEKLTQDEIISKTKQVIQGLEALKNEHNSILQSLLETLKCLKKDDESNLVEEKSNMIRKSLEMLELGLSEAQVMMALSNHLNAVESEKQKLRAQVRRLCQENQWLRDELANTQQKLQKSEQSVAQLEEEKKHLEFMNQLKKYDDDISPSEDKDTDSTKEPLDDLFPNDEDDPGQGIQQQHSSAAAAAQQGGYEIPARLRTLHNLVIQYASQGRYEVAVPLCKQALEDLEKTSGHDHPDVATMLNILALVYRDQNKYKDAANLLNDALAIREKTLGKDHPAVAATLNNLAVLYGKRGKYKEAEPLCKRALEIREKVLGKDHPDVAKQLNNLALLCQNQGKYEEVEYYYQRALEIYQTKLGPDDPNVAKTKNNLASCYLKQGKFKQAETLYKEILTRAHEREFGSVDDENKPIWMHAEEREECKGKQKDGTSFGEYGGWYKACKVDSPTVTTTLKNLGALYRRQGKFEAAETLEEAAMRSRKQGLDNVHKQRVAEVLNDPENMEKRRSRESLNVDVVKYESGPDGGEEVSMSVEWNGGVSGRASFCGKRQQQQWPGRRHR

SEQ ID NO: 8 - protein, 400 aa, Homo Sapiens
MNGQLDLSGKLIIKAQLGEDIRRIPIHNEDITYDELVLMMQRVFRGKLLSNDEVTIKYKDEDGDLITIFDSSDLSFAIQCSRILKLTLFVNGQPRPLESSQVKYLRRELIELRNKVNRLLDSLEPPGEPGPSTNIPENDTVDGREEKSASDSSGKQSTQVMAASMSAFDPLKNQDEINKNVMSAFGLTDDQVSGPPSAPAEDRSGTPDSIASSSSAAHPPGVQPQQPPYTGAQTQAGQIEGQMYQQYQQQAGYGAQQPQAPPQQPQQYGIQYSASYSQQTGPQQPQQFQGYGQQPTSQAPAPAFSGQPQQLPAQPPQQYQASNYPAQTYTAQTSQPTNYTVAPASQPGMAPSQPGAYQPRPGFTSLPGSTMTPPPSGPNPYARNRPPFGQGYTQPGPGYR

SEQ ID NO: 9 - protein, 2363 aa, Homo Sapiens
MAAVLQQVLERTELNKLPKSVQNKLEKFLADQQSEIDGLKGRHEKFKVESEQQYFEIEKRLSHSQERLVNETRECQSLRLELEKLNNQLKALTEKNKELEIAQDRNIAIQSQFTRTKEELEAEKRDLIRTNERLSQELEYLTEDVKRLNEKLKESNTTKGELQLKLDELQASDVSVKYREKRLEQEKELLHSQNTWLNTELKTKTDELLALGREKGNEILELKCNLENKKEEVSRLEEQMNGLKTSNEHLQKHVEDLLTKLKEAKEQQASMEEKFHNELNAHIKLSNLYKSAADDSEAKSNELTRAVEELHKLLKEAGEANKAIQDHLLEVEQSKDQMEKEMLEKIGRLEKELENANDLLSATKRKGAILSEEELAAMSPTAAAVAKIVKPGMKLTELYNAYVETQDQLLLEKLENKRINKYLDEIVKEVEAKAPILKRQREEYERAQKAVASLSVKLEQAMKEIQRLQEDTDKANKQSSVLERDNRRMEIQVKDLSQQIRVLLMELEEARGNHVIRDEEVSSADISSSSEVISQHLVSYRNIEELQQQNQRLLVALRELGETREREEQETTSSKITELQLKLESALTELEQLRKSRQHQMQLVDSIVRQRDMYRILLSQTTGVAIPLHASSLDDVSLASTPKRPSTSQTVSTPAPVPVIESTEAIEAKAALKQLQEIFENYKKEKAENEKIQNEQLEKLQEQVTDLRSQNTKISTQLDFASKRYEMLQDNVEGYRREITSLHERNQKLTATTQKQEQIINTMTQDLRGANEKLAVAEVRAENLKKEKEMLKLSEVRLSQQRESLLAEQRGQNLLLTNLQTIQGILERSETETKQRLSSQIEKLEHEISHLKKKLENEVEQRHTLTRNLDVQLLDTKRQLDTETNLHLNTKELLKNAQKEIATLKQHLSNMEVQVASQSSQRTGKGQPSNKEDVDDLVSQLRQTEEQVNDLKERLKTSTSNVEQYQAMVTSLEESLNKEKQVTEEVRKNIEVRLKESAEFQTQLEKKLMEVEKEKQELQDDKRRAIESMEQQLSELKKTLSSVQNEVQEALQRASTALSNEQQARRDCQEQAKIAVEAQNKYERELMLHAADVEALQAAKEQVSKMASVRQHLEETTQKAESQLLECKASWEERERMLKDEVSKCVCRCEDLEKQNRLLHDQIEKLSDKVVASVKEGVQGPLNVSLSEEGKSQEQILEILRFIRREKEIAETRFEVAQVESLRYRQRVELLERELQELQDSLNAEREKVQVTAKTMAQHEELMKKTETMNVVMETNKMLREEKERLEQDLQQMQAKVRKLELDILPLQEANAELSEKSGMLQAEKKLLEEDVKRWKARNQHLVSQQKDPDTEEYRKLLSEKEVHTKRIQQLTEEIGRLKAEIARSNASLTNNQNLIQSLKEDLNKVRTEKETIQKDLDAKIIDIQEKVKTITQVKKIGRRYKTQYEELKAQQDKVMETSAQSSGDHQEQHVSVQEMQELKETLNQAETKSKSLESQVENLQKTLSEKETEARNLQEQTVQLQSELSRLRQDLQDRTTQEEQLRQQITEKEEKTRKAIVAAKSKIAHLAGVKDQLTKENEELKQRNGALDQQKDELDVRITALKSQYEGRISRLERELREHQERHLEQRDEPQEPSNKVPEQQRQITLKTTPASGERGIASTSDPPTANIKPTPVVSTPSKVTAAAMAGNKSTPRASIRPMVTPATVTNPTTTPTATVMPTTQVESQEAMQSEGPVEHVPVFGSTSGSVRSTSPNVQPSISQPILTVQQQTQATAFVQPTQQSHPQIEPANQELSSNIVEVVQSSPVERPSTSTAVFGTVSATPSSSLPKRTREEEEDSTIEASDQVSDDTVEMPLPKKLKSVTPVGTEEEVMAEESTDGEVETQVYNQDSQDSIGEGVTQGDYTPMEDSEETSQSLQIDLGPLQSDQQTTTSSQDGQGKGDDVIVIDSDDEEEDDDENDGEHEDYEEDEEDDDDDEDDTGMGDEGEDSNEGTGSADGNDGYEADDAEGGDGTDPGTETEESMGGGEGNHRAADSQNSGEGNTGAAESSFSQEVSREQQPSSASERQAPRAPQSPRRPPHPLPPRLTIHAPPQELGPPVQRIQMTRRQSVGRGLQLTPGIGGMQQHFFDDEDRTVPSTPTLVVPHRTDGFAEAIHSPQVAGVPRFRFGPPEDMPQTSSSHSDLGQLASQGGLGMYETPLFLAHEEESGGRSVPTTPLQVAAPVTVFTESTTSDASEHASQSVPMVTTSTGTLSTTNETATGDDGDEVFVEAESEGISSEAGLEIDSQQEEEPVQASDESDLPSTSQDPPSSSSVDTSSSQPKPFRRVRLQTTLRQGVRGRQFNRQRGVSHAMGGRGGINRGNIN

SEQ ID NO: 10 - protein, 1037 aa, Homo Sapiens
MDRMASSMKQVPNPLPKVLSRRGVGAGLEAAERESFERTQTVSINKAINTQEVAVKEKHARTCILGTHHEKGAQTFWSVVNRLPLSSNAVLCWKFCHVFHKLLRDGHPNVLKDSLRYRNELSDMSRMWGHLSEGYGQLCSIYLKLLRTKMEYHTKNPRFPGNLQMSDRQLDEAGESDVNNFFQLTVEMFDYLECELNLFQTVFNSLDMSRSVSVTAAGQCRLAPLIQVILDCSHLYDYTVKLLFKLHSCLPADTLQGHRDRFMEQFTKLKDLFYRSSNLQYFKRLIQIPQLPENPPNFLRASALSEHISPVVVIPAEASSPDSEPVLEKDDLMDMDASQQNLFDNKFDDIFGSSFSSDPFNFNSQNGVNKDEKDHLIERLYREISGLKAQLENMKTESQRVVLQLKGHVSELEADLAEQQHLRQQAADDCEFLRAELDELRRQREDTEKAQRSLSEIERKAQANEQRYSKLKEKYSELVQNHADLLRKNAEVTKQVSMARQAQVDLEREKKELEDSLERISDQGQRKTQEQLEVLESLKQELATSQRELQVLQGSLETSAQSEANWAAEFAELEKERDSLVSGAAHREEELSALRKELQDTQLKLASTEESMCQLAKDQRKMLLVGSRKAAEQVIQDALNQLEEPPLISCAGSADHLLSTVTSISSCIEQLEKSWSQYLACPEDISGLLHSITLLAHLTSDAIAHGATTCLRAPPEPADSLTEACKQYGRETLAYLASLEEEGSLENADSTAMRNCLSKIKAIGEELLPRGLDIKQEELGDLVDKEMAATSAAIETATARIEEMLSKSRAGDTGVKLEVNERILGCCTSLMQAIQVLIVASKDLQREIVESGRGTASPKEFYAKNSRWTEGLISASKAVGWGATVMVDAADLVVQGRGKFEELMVCSHEIAASTAQLVAASKVKADKDSPNLAQLQQASRGVNQATAGVVASTISGKSQIEETDNMDFSSMTLTQIKRQEMDSQVRVLELENELQKERQKLGELRKKHYELAGVAEGWEEGTEASPPTLQEVVTEKE

SEQ ID NO: 11 - protein, 780 aa, Homo Sapiens
MDEQAGPGVFFSNNHPGAGGAKGLGPLAEAAAAGDGAAAAGAARAQYSLPGILHFLQHEWARFEVERAQWEVERAELQAQIAFLQGERKGQENLKKDLVRRIKMLEYALKQERAKYHKLKYGTELNQGDMKPPSYDSDEGNETEVQPQQNSQLMWKQGRQLLRQYLQEVGYTDTILDVKSKRVRALLGFSSDVTDREDDKNQDSVVNGTEAEVKETAMIAKSELTDSASVLDNFKFLESAAADFSDEDEDDDVDGREKSVIDTSTIVRKKALPDSGEDRDTKEALKEFDFLVTSEEGDNESRSAGDGTDWEKEDQCLMPEAWNVDQGVITKLKEQYKKERKGKKGVKRPNRSKLQDMLANLRDVDELPSLQPSVGSPSRPSSSRLPEHEINRADEVEALTFPPSSGKSFIMGADEALESELGLGELAGLTVANEADSLTYDIANNKDALRKTWNPKFTLRSHFDGIRALAFHPIEPVLITASEDHTLKMWNLQKTAPAKKSTSLDVEPIYTFRAHKGPVLCVVMSSNGEQCYSGGTDGLIQGWNTTNPNIDPYDSYDPSVLRGPLLGHTDAVWGLAYSAAHQRLLSCSADGTLRLWNTTEVAPALSVFNDTKELGIPASVDLVSSDPSHMVASFSKGYTSIFNMETQQRILTLESNVDTTANSSCQINRVISHPTLPISITAHEDRHIKFYDNNTGKLIHSMVAHLEAVTSLAVDPNGLYLMSGSHDCSIRLWNLESKTCIQEFTAHRKKFEESIHDVAFHPSKCYIASAGADALAKVFV

SEQ ID NO: 12 - protein, 1278 aa, Homo Sapiens
MAQSKRHVYSRTPSGSRMSAEASARPLRVGSRVEVIGKGHRGTVAYVGATLFATGKWVGVILDEAKGKNDGTVQGRKYFTCDEGHGIFVRQSQIQVFEDGADTTSPETPDSSASKVLKREGTDTTAKTSKLRGLKPKKAPTARKTTTRRPKPTRPASTGVAGASSSLGPSGSASAGELSSSEPSTPAQTPLAAPIIPTPVLTSPGAVPPLPSPSKEEEGLRAQVRDLEEKLETLRLKRAEDKAKLKELEKHKIQLEQVQEWKSKMQEQQADLQRRLKEARKEAKEALEAKERYMEEMADTADAIEMATLDKEMAEERAESLQQEVEALKERVDELTTDLEILKAEIEEKGSDGAASSYQLKQLEEQNARLKDALVRMRDLSSSEKQEHVKLQKLMEKKNQELEVVRQQRERLQEELSQAESTIDELKEQVDAALGAEEMVEMLTDRNLNLEEKVRELRETVGDLEAMNEMNDELQENARETELELREQLDMAGARVREAQKRVEAAQETVADYQQTIKKYRQLTAHLQDVNRELTNQQEASVERQQQPPPETFDFKIKFAETKAHAKAIEMELRQMEVAQANRHMSLLTAFMPDSFLRPGGDHDCVLVLLLMPRLICKAELIRKQAQEKFELSENCSERPGLRGAAGEQLSFAAGLVYSLSLLQATLHRYEHALSQCSVDVYKKVGSLYPEMSAHERSLDFLIELLHKDQLDETVNVEPLTKAIKYYQHLYSIHLAEQPEDCTMQLADHIKFTQSALDCMSVEVGRLRAFLQGGQEATDIALLLRDLETSCSDIRQFCKKIRRRMPGTDAPGIPAALAFGPQVSDTLLDCRKHLTWVVAVLQEVAAAAAQLIAPLAENEGLLVAALEELAFKASEQIYGTPSSSPYECLRQSCNILISTMNKLATAMQEGEYDAERPPSKPPPVELRAAALRAEITDAEGLGLKLEDRETVIKELKKSLKIKGEELSEANVRLSLLEKKLDSAAKDADERIEKVQTRLEETQALLRKKEKEFEETMDALQADIDQLEAEKAELKQRLNSQSKRTIEGLRGPPPSGIATLVSGIAGEEQQRGAIPGQAPGSVPGPGLVKDSPLLLQQISAMRLHISQLQHENSILKGAQMKASLASLPPLHVAKLSHEGPGSELPAGALYRKTSQLLETLNQLSTHTHVVDITRTSPAAKSPSAQLMEQVAQLKSLSDTVEKLKDEVLKETVSQRPGATVPTDFATFPSSAFLRAKEEQQDDTVYMGKVTFSCAAGFGQRHRLVLTQEQLHQLHSRLIS

SEQ ID NO: 13 - protein, 440 aa, Homo Sapiens
MASLTVKAYLLGKEDAAREIRRFSFCCSPEPEAEAEAAAGPGPCERLLSRVAALFPALRPGGFQAHYRDEDGDLVAFSSDEELTMAMSYVKDDIFRIYIKEKKECRRDHRPPCAQEAPRNMVHPNVICDGCNGPVVGTRYKCSVCPDYDLCSVCEGKGLHRGHTKLAFPSPFGHLSEGFSHSRWLRKVKHGHFGWPGWEMGPPGNWSPRPPRAGEARPGPTAESASGPSEDPSVNFLKNVGESVAAALSPLGIEVDIDVEHGGKRSRLTPVSPESSSTEEKSSSQPSSCCSDPSKPGGNVEGATQSLAEQMRKIALESEGRPEEQMESDNCSGGDDDWTHLSSKEVDPSTGELQSLQMPESEGPSSLDPSQEGPTGLKEAALYPHLPPEADPRLIESLSQMLSMGFSDEGGWLTRLLQTKNYDIGAALDTIQYSKHPPPL

SEQ ID NO: 14 - protein, 294 aa, Homo Sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 15 - protein, 835 aa, Homo Sapiens
MSRRKQGKPQHLSKREFSPEPLEAILTDDEPDHGPLGAPEGDHDLLTCGQCQMNFPLGDILIFIEHKRKQCNGSLCLEKAVDKPPSPSPIEMKKASNPVEVGIQVTPEDDDCLSTSSRGICPKQEHIADKLLHWRGLSSPRSAHGALIPTPGMSAEYAPQGICKDEPSSYTCTTCKQPFTSAWFLLQHAQNTHGLRIYLESEHGSPLTPRVGIPSGLGAECPSQPPLHGIHIADNNPFNLLRIPGSVSREASGLAEGRFPPTPPLFSPPPRHHLDPHRIERLGAEEMALATHHPSAFDRVLRLNPMAMEPPAMDFSRRLRELAGNTSSPPLSPGRPSPMQRLLQPFQPGSKPPFLATPPLPPLQSAPPPSQPPVKSKSCEFCGKTFKFQSNLVVHRRSHTGEKPYKCNLCDHACTQASKLKRHMKTHMHKSSPMTVKSDDGLSTASSPEPGTSDLVGSASSALKSVVAKFKSENDPNLIPENGDEEEEEDDEEEEEEEEEEEEELTESERVDYGFGLSLEAARHHENSSRGAVVGVGDESRALPDVMQGMVLSSMQHFSEAFHQVLGEKHKRGHLAEAEGHRDTCDEDSVAGESDRIDDGTVNGRGCSPGESASGGLSKKLLLGSPSSLSPFSKRIKLEKEFDLPPAAMPNTENVYSQWLAGYAASRQLKDPFLSFGDSRQSPFASSSEHSSENGSLRFSTPPGELDGGISGRSGTGSGGSTPHISGPGPGRPSSKEGRRSDTCEYCGKVFKNCSNLTVHRRSHTGERPYKCELCNYACAQSSKLTRHMKTHGQVGKDVYKCEICKMPFSVYSTLEKHMKKWHSDRVLNNDIKTE

SEQ ID NO: 16 - protein, 4857 aa, Homo Sapiens
MVTGGGAAPPGTVTEPLPSVIVLSAGRKMAAAAAAASGPGCSSAAGAGAAGVSEWLVLRDGCMHCDADGLHSLSYHPALNAILAVTSRGTIKVIDGTSGATLQASALSAKPGGQVKCQYISAVDKVIFVDDYAVGCRKDLNGILLLDTALQTPVSKQDDVVQLELPVTEAQQLLSACLEKVDISSTEGYDLFITQLKDGLKNTSHETAANHKVAKWATVTFHLPHHVLKSIASAIVNELKKINQNVAALPVASSVMDRLSYLLPSARPELGVGPGRSVDRSLMYSEANRRETFTSWPHVGYRWAQPDPMAQAGFYHQPASSGDDRAMCFTCSVCLVCWEPTDEPWSEHERHSPNCPFVKGEHTQNVPLSVTLATSPAQFPCTDGTDRISCFGSGSCPHFLAAATKRGKICIWDVSKLMKVHLKFEINAYDPAIVQQLILSGDPSSGVDSRRPTLAWLEDSSSCSDIPKLEGDSDDLLEDSDSEEHSRSDSVTGHTSQKEAMEVSLDITALSILQQPEKLQWEIVANVLEDTVKDLEELGANPCLTNSKSEKTKEKHQEQHNIPFPCLLAGGLLTYKSPATSPISSNSHRSLDGLSRTQGESISEQGSTDNESCTNSELNSPLVRRTLPVLLLYSIKESDEKAGKIFSQMNNIMSKSLHDDGFTVPQIIEMELDSQEQLLLQDPPVTYIQQFADAAANLTSPDSEKWNSVFPKPGTLVQCLRLPKFAEEENLCIDSITPCADGIHLLVGLRTCPVESLSAINQVEALNNLNKLNSALCNRRKGELESNLAVVNGANISVIQHESPADVQTPLIIQPEQRNVSGGYLVLYKMNYATRIVTLEEEPIKIQHIKDPQDTITSLILLPPDILDNREDDCEEPIEDMQLTSKNGFEREKTSDISTLGHLVITTQGGYVKILDLSNFEILAKVEPPKKEGTEEQDTFVSVIYCSGTDRLCACTKGGELHFLQIGGTCDDIDEADILVDGSLSKGIEPSSEGSKPLSNPSSPGISGVDLLVDQPFTLEILTSLVELTRFETLTPRFSATVPPCWVEVQQEQQQRRHPQHLHQQHHGDAAQHTRTWKLQTDSNSWDEHVFELVLPKACMVGHVDFKFVLNSNITNIPQIQVTLLKNKAPGLGKVNALNIEVEQNGKPSLVDLNEEMQHMDVEESQCLRLCPFLEDHKEDILCGPVWLASGLDLSGHAGMLTLTSPKLVKGMAGGKYRSFLIHVKAVNERGTEEICNGGMRPVVRLPSLKHQSNKGYSLASLLAKVAAGKEKSSNVKNENTSGTRKSENLRGCDLLQEVSVTIRRFKKTSISKERVQRCAMLQFSEFHEKLVNTLCRKTDDGQITEHAQSLVLDTLCWLAGVHSNGPGSSKEGNENLLSKTRKFLSDIVRVCFFEAGRSIAHKCARFLALCISNGKCDPCQPAFGPVLLKALLDNMSFLPAATTGGSVYWYFVLLNYVKDEDLAGCSTACASLLTAVSRQLQDRLTPMEALLQTRYGLYSSPFDPVLFDLEMSGSSCKNVYNSSIGVQSDEIDLSDVLSGNGKVSSCTAAEGSFTSLTGLLEVEPLHFTCVSTSDGTRIERDDAMSSFGVTPAVGGLSSGTVGEASTALSSAAQVALQSLSHAMASAEQQLQVLQEKQQQLLKLQQQKAKLEAKLHQTTAAAAAAASAVGPVHNSVPSNPVAAPGFFIHPSDVIPPTPKTTPLFMTPPLTPPNEAVSVVINAELAQLFPGSVIDPPAVNLAAHNKNSNKSRMNPLGSGLALAISHASHFLQPPPHQSIIIERMHSGARRFVTLDFGRPILLTDVLIPTCGDLASLSIDIWTLGEEVDGRRLVVATDISTHSLILHDLIPPPVCRFMKITVIGRYGSTNARAKIPLGFYYGHTYILPWESELKLMHDPLKGEGESANQPEIDQHLAMMVALQEDIQCRYNLACHRLETLLQSIDLPPLNSANNAQYFLRKPDKAVEEDSRVFSAYQDCIQLQLQLNLAHNAVQRLKVALGASRKMLSETSNPEDLIQTSSTEQLRTIIRYLLDTLLSLLHASNGHSVPAVLQSTFHAQACEELFKHLCISGTPKIRLHTGLLLVQLCGGERWWGQFLSNVLQELYNSEQLLIFPQDRVFMLLSCIGQRSLSNSGVLESLLNLLDNLLSPLQPQLPMHRRTEGVLDIPMISWVVMLVSRLLDYVATVEDEAAAAKKPLNGNQWSFINNNLHTQSLNRSSKGSSSLDRLYSRKIRKQLVHHKQQLNLLKAKQKALVEQMEKEKIQSNKGSSYKLLVEQAKLKQATSKHFKDLIRLRRTAEWSRSNLDTEVTTAKESPEIEPLPFTLAHERCISVVQKLVLFLLSMDFTCHADLLLFVCKVLARIANATRPTIHLCEIVNEPQLERLLLLLVGTDFNRGDISWGGAWAQYSLTCMLQDILAGELLAPVAAEAMEEGTVGDDVGATAGDSDDSLQQSSVQLLETIDEPLTHDITGAPPLSSLEKDKEIDLELLQDLMEVDIDPLDIDLEKDPLAAKVFKPISSTWYDYWGADYGTYNYNPYIGGLGIPVAKPPANTEKNGSQTVSVSVSQALDARLEVGLEQQAELMLKMMSTLEADSILQALTNTSPTLSQSPTGTDDSLLGGLQAANQTSQLIIQLSSVPMLNVCFNKLFSMLQVHHVQLESLLQLWLTLSLNSSSTGNKENGADIFLYNANRIPVISLNQASITSFLTVLAWYPNTLLRTWCLVLHSLTLMTNMQLNSGSSSAIGTQESTAHLLVSDPNLIHVLVKFLSGTSPHGTNQHSPQVGPTATQAMQEFLTRLQVHLSSTCPQIFSEFLLKLIHILSTERGAFQTGQGPLDAQVKLLEFTLEQNFEVVSVSTISAVIESVTFLVHHYITCSDKVMSRSGSDSSVGARACFGGLFANLIRPGDAKAVCGEMTRDQLMFDLLKLVNILVQLPLSGNREYSARVSVTTNTTDSVSDEEKVSGGKDGNGSSTSVQGSPAYVADLVLANQQIMSQILSALGLCNSSAMAMIIGASGLHLTKHENFHGGLDAISVGDGLFTILTTLSKKASTVHMMLQPILTYMACGYMGRQGSLATCQLSEPLLWFILRVLDTSDALKAFHDMGGVQLICNNMVTSTRAIVNTARSMVSTIMKFLDSGPNKAVDSTLKTRILASEPDNAEGIHNFAPLGTITSSSPTAQPAEVLLQATPPHRRARSAAWSYIFLPEEAWCDLTIHLPAAVLLKEIHIQPHLASLATCPSSVSVEVSADGVNMLPLSTPVVTSGLTYIKIQLVKAEVASAVCLRLHRPRDASTLGLSQIKLLGLTAFGTTSSATVNNPFLPSEDQVSKTSIGWLRLLHHCLTHISDLEGMMASAAAPTANLLQTCAALLMSPYCGMHSPNIEVVLVKIGLQSTRIGLKLIDILLRNCAASGSDPTDLNSPLLFGRLNGLSSDSTIDILYQLGTTQDPGTKDRIQALLKWVSDSARVAAMKRSGRMNYMCPNSSTVEYGLLMPSPSHLHCVAAILWHSYELLVEYDLPALLDQELFELLFNWSMSLPCNMVLKKAVDSLLCSMCHVHPNYFSLLMGWMGITPPPVQCHHRLSMTDDSKKQDLSSSLTDDSKNAQAPLALTESHLATLASSSQSPEAIKQLLDSGLPSLLVRSLASFCFSHISSSESIAQSIDISQDKLRRHHVPQQCNKMPITADLVAPILRFLTEVGNSHIMKDWLGGSEVNPLWTALLFLLCHSGSTSGSHNLGAQQTSARSASLSSAATTGLTTQQRTAIENATVAFFLQCISCHPNNQKLMAQVLCELFQTSPQRGNLPTSGNISGFIRRLFLQLMLEDEKVTMFLQSPCPLYKGRINATSHVIQHPMYGAGHKFRTLHLPVSTTLSDVLDRVSDTPSITAKLISEQKDDKEKKNHEEKEKVKAENGFQDNYSVVVASGLKSQSKRAVSATPPRPPSRRGRTIPDKIGSTSGAEAANKIITVPVFHLFHKLLAGQPLPAEMTLAQLLTLLYDRKLPQGYRSIDLTVKLGSRVITDPSLSKTDSYKRLHPEKDHGDLLASCPEDEALTPGDECMDGILDESLLETCPIQSPLQVFAGMGGLALIAERLPMLYPEVIQQVSAPVVTSTTQEKPKDSDQFEWVTIEQSGELVYEAPETVAAEPPPIKSAVQTMSPIPAHSLAAFGLFLRLPGYAEVLLKERKHAQCLLRLVLGVTDDGEGSHILQSPSANVLPTLPFHVLRSLFSTTPLTTDDGVLLRRMALEIGALHLILVCLSALSHHSPRVPNSSVNQTEPQVSSSHNPTSTEEQQLYWAKGTGFGTGSTASGWDVEQALTKQRLEEEHVTCLLQVLASYINPVSSAVNGEAQSSHETRGQNSNALPSVLLELLSQSCLIPAMSSYLRNDSVLDMARHVPLYRALLELLRAIASCAAMVPLLLPLSTENGEEEEEQSECQTSVGTLLAKMKTCVDTYTNRLRSKRENVKTGVKPDASDQEPEGLTLLVPDIQKTAEIVYAATTSLRQANQEKKLGEYSKKAAMKPKPLSVLKSLEEKYVAVMKKLQFDTFEMVSEDEDGKLGFKVNYHYMSQVKNANDANSAARARRLAQEAVTLSTSLPLSSSSSVFVRCDEERLDIMKVLITGPADTPYANGCFEFDVYFPQDYPSSPPLVNLETTGGHSVRFNPNLYNDGKVCLSILNTWHGRPEEKWNPQTSSFLQVLVSVQSLILVAEPYFNEPGYERSRGTPSGTQSSREYDGNIRQATVKWAMLEQIRNPSPCFKEVIHKHFYLKRVEIMAQCEEWIADIQQYSSDKRVGRTMSHHAAALKRHTAQLREELLKLPCPEGLDPDTDDAPEVCRATTGAEETLMHDQVKPSSSKELPSDFQL

SEQ ID NO: 17 - protein, 116 aa, Homo Sapiens
EVQLLESGGGLVQPGGSLRLSCAASGFTFSSYAMNWVRQAPGKGLEWVSTTSGSGASTYYADSVKGRFTISRDNSKNTLYLQMNSLRAEDTAVYYCAKIWIAFDIWGQGTMVTVSS

SEQ ID NO: 18 - protein, 108 aa, Homo Sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIK

SEQ ID NO: 19 - protein, 8 aa, Homo Sapiens
GFTFSSYA

SEQ ID NO: 20 - protein, 8 aa, Homo Sapiens
TSGSGAST

SEQ ID NO: 21 - protein, 9 aa, Homo Sapiens
AKIWIAFDI

SEQ ID NO: 22 - protein, 7 aa, Homo Sapiens
QSVSSSY

SEQ ID NO: 23 - protein, 9 aa, Homo Sapiens
QQYGSSPYT

(19) United States
(12) Patent Application Publication
(10) Pub. No.: US 2021/0393793 A1
(43) Pub. Date: Dec. 23, 2021

(54) AXL-SPECIFIC ANTIBODIES FOR TREATMENT OF NON-SMALL CELL LUNG CANCER

(71) Applicant: GENMAB A/S, Copenhagen V (DK)

(72) Inventors: Esther BREIJ, Utrecht (NL); Ulf FORSSMANN, Hannover (DE); Tahamtan AHMADI, Rydal, PA (US)

(21) Appl. No.: 17/279,359

(22) PCT Filed: Sep. 26, 2019
(86) PCT No.: PCT/IB2019/001040

§ 371 Date: Mar. 24, 2021

Related U.S. Application Data
(60) Provisional application No. 62/804,551, filed on Feb. 12, 2019.
(60) Provisional application No. 62/736,950, filed on Sep. 26, 2018.

(51) Int. Cl. A61K 47/68; C07K 16/28; A61P 35/00; A61K 9/00

(52) U.S. Cl. CPC A61K 47/6857; A61K 47/6803; C07K 16/2863; A61K 2039/505; A61K 9/0019; C07K 2317/565; A61P 35/00

(57) ABSTRACT

The present invention relates to immunoconjugates anti-AXL antibodies and compositions for treatment of non-small cell lung cancer.

### FIELD OF INVENTION

The present invention relates to the use of immunoconjugates of antibodies binding AXL, and compositions comprising such immunoconjugates for treatment of non-small cell lung cancer; in particular for the treatment of non-small cell lung cancer.

### BACKGROUND

AXL is a 104-140 kDa transmembrane protein which belongs to the TAM subfamily of mammalian Receptor Tyrosine Kinases (RTKs) and which has transforming abilities. The AXL extracellular domain is composed of a combination of two membrane-distal N-terminal immunoglobulin (Ig)-like domains (Ig1 and Ig2 domains) and two membrane-proximal fibronectin type III (FNIII) repeats (the FN1- and FN2-domains). Enhanced or de novo expression of AXL has been reported in a variety of cancers, including gastric, prostate, ovarian, and lung cancer.

AXL can be activated upon binding of its ligand, the vitamin K-dependent growth arrest-specific factor 6 (Gas6). Gas6-binding to AXL leads to AXL dimerization, autophosphorylation and subsequent activation of intracellular signaling pathways, such as the PI3K/AKT, mitogen-activated protein kinase (MAPK), STAT and NF-κB cascades. In cancer cells, AXL expression has been associated with tumor cell motility, invasion, migration, and is involved in epithelial-to-mesenchymal transition (EMT).

Targeted inhibition of AXL and/or its ligand Gas6 may be effective as anti-tumor therapy using, e.g., small molecules or anti-AXL antibodies. Anti-AXL antibodies have been described that attenuate NSCLC and breast cancer xenograft growth in vivo by downregulation of receptor expression, reducing tumor cell proliferation and inducing apoptosis. Various other anti-AXL antibodies have also been reported, including an ADC based on an anti-AXL antibody and a pyrrolobenzo-diazepine (PBD) dimer.

Lung cancer is the most frequently diagnosed cancer and also the most common cause of cancer mortality. Lung cancer is classified according to the World Health Organization depending on its origin. The majority of lung cancers arise from the bronchial epithelium and include Non-small cell lung cancer (NSCLC) and Small-cell lung cancer and account for approximately 90% of all lung cancers, whereas the remaining are of different origin, e.g., mesotheliomas, lymphomas and stromal tumors. NSCLC represents approximately 80% of lung cancer and includes adenocarcinomas which account for approximately 50% of lung cancers, squamous cell carcinomas (SCC) which account for approximately 20% and large cell carcinomas which account for approximately 10% of lung cancers.

In the US, 133,703 new cases of NSCLC were diagnosed in 2014′ and 211,401 patients were diagnosed in the EU in 2012. Sixty-six percent of the patients had advanced or metastatic disease (stage IIIA, IIIB or IV) at the time of diagnosis and the 5-years overall survival was 17% in 2014.

There remain a need for improved methods of treating cancers, including NSCLC.

### SUMMARY OF THE INVENTION

It is an object of the present invention to provide a conjugate of monomethyl auristatin or a functional analog or derivative thereof and an antibody or antigen-binding fragment thereof capable of binding to human Axl (SEQ ID NO: 1), comprising
a heavy chain variable (VH) region comprising the CDR1, CDR2, and CDR3 sequences of SEQ ID Nos.: 19, 20, and 21, respectively, and
a light chain variable (VL) region comprising the CDR1, CDR2, and CDR3 sequences of SEQ ID Nos.: 22, GAS, and 23, respectively;

for use in treating cancer in a subject, wherein

the cancer is non-small cell lung cancer (NSCLC), and

the conjugate is administered to the subject at a dose of about 1.8-about 2.6 mg/kg body weight once every three weeks or by weekly dosing of about 0.8-about 1.2 mg/kg body weight for three weeks, optionally followed by one treatment-free week.

Another aspect of the invention provides a method of treating a cancer in a subject, the method comprising administering to the subject a conjugate of monomethyl auristatin or a functional analog or derivative thereof and an antibody or antigen-binding fragment thereof capable of binding to human Axl (SEQ ID NO: 1), comprising
a heavy chain variable (VH) region comprising the CDR1, CDR2, and CDR3 sequences of SEQ ID Nos.: 19, 20, and 21, respectively, and
a light chain variable (VL) region comprising the CDR1, CDR2, and CDR3 sequences of SEQ ID Nos.: 22, GAS, and 23, respectively; wherein

the cancer is non-small cell lung cancer (NSCLC), and

the conjugate is administered to the subject at a dose of about 1.8-about 2.6 mg/kg body weight once every three weeks or by weekly dosing of about 0.8-about 1.2 mg/kg body weight for three weeks, optionally followed by one treatment-free week.

Finally, the invention provides a kit comprising a conjugate of monomethyl auristatin or a functional analog or derivative thereof and an antibody or antigen-binding fragment thereof capable of binding to human Axl (SEQ ID NO: 1), comprising
a heavy chain variable (VH) region comprising the CDR1, CDR2, and CDR3 sequences of SEQ ID Nos.: 19, 20, and 21, respectively, and
a light chain variable (VL) region comprising the CDR1, CDR2, and CDR3 sequences of SEQ ID Nos.: 22, GAS, and 23, respectively;

and instructions for using the conjugate according to the invention and as disclosed herein.

### DRAWINGS

FIG. 1: Design of phase 2 study including dose excalation and expansion.

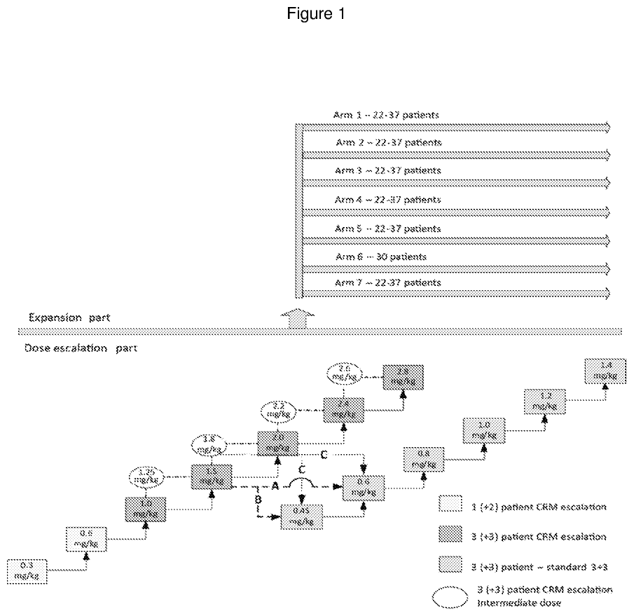

FIG. 2: Design of 1Q3W dosage regimen: Dosing once every 3 weeks.

FIG. 3: Design of 3Q4W dosage regimen: Weekly dosing for 3 weeks followed by one treatment-free week.

FIG. 4: Subject 403 lesion snapshots.

### DETAILED DESCRIPTION

### Definitions

In a first aspect the present invention provides an antibody binding to human AXL or an antibody-drug conjugate (ADC) comprising an antibody binding to human AXL as defined in any aspect or embodiment herein, for use in treating cancer in a subject. In particular the antibody or ADC is for use in treating cancer in which prior treatment has not been effective.

The term “AXL” or “Axl” as used herein, refers to the protein entitled AXL, which is also referred to as UFO or JTK11, a 894 amino acid protein with a molecular weight of 104-140 kDa that is part of the subfamily of mammalian TAM Receptor Tyrosine Kinases (RTKs). The molecular weight is variable due to potential differences in glycosylation of the protein. The AXL protein consists of two extracellular immunoglobulin-like (Ig-like) domains on the N-terminal end of the protein, two membrane-proximal extracellular fibronectin type III (FNIII) domains, a transmembrane domain and an intracellular kinase domain. AXL is activated upon binding of its ligand Gas6, by ligand-independent homophilic interactions between AXL extracellular domains, by autophosphorylation in presence of reactive oxygen species or by transactivation through EGFR (Meyer et al., 2013), and is aberrantly expressed in several tumor types. In humans, the AXL protein is encoded by a nucleic acid sequence encoding the amino acid sequence shown in SEQ ID NO: 1 (human AXL protein: Swissprot P30530). For cynomolgus AXL protein, see Genbank accession HB387229.1 (SEQ ID NO: 2).

The term “antibody” as used herein is intended to refer to an immunoglobulin molecule, a fragment of an immunoglobulin molecule, or a derivative of either thereof, which has the ability to specifically bind to an antigen under typical physiological and/or tumor-specific conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to be internalized). The binding region (or binding domain which may be used herein, both having the same meaning) which interacts with an antigen, comprises variable regions of both the heavy and light chains of the immunoglobulin molecule. The constant regions of the antibodies (Abs) may mediate the binding of the immunoglobulin to host tissues or factors, including various cells of the immune system (such as effector cells) and components of the complement system such as C1q, the first component in the classical pathway of complement activation. As indicated above, the term antibody as used herein, unless otherwise stated or clearly contradicted by context, includes fragments of an antibody that retain the ability to specifically interact, such as bind, to the antigen. It has been shown that the antigen-binding function of an antibody may be performed by fragments of a full-length antibody. Examples of binding fragments encompassed within the term “antibody” include (i) a Fab′ or Fab fragment, a monovalent fragment consisting of the VL, VH, CL and CH1 domains, or a monovalent antibody as described in WO 2007/059782; (ii) F(ab′)2 fragments, bivalent fragments comprising two Fab fragments linked by a disulfide bridge at the hinge region; (iii) an Fd fragment consisting essentially of the VH and CH1 domains; (iv) an Fv fragment consisting essentially of the VL and VH domains of a single arm of an antibody, (v) a dAb fragment, which consists essentially of a VH domain and is also called domain antibody; (vi) camelid or nanobodies and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. These and other useful antibody fragments in the context of the present invention are discussed further herein. It also should be understood that the term antibody, unless specified otherwise, also includes polyclonal antibodies, monoclonal antibodies (mAbs), antibody-like polypeptides, such as chimeric antibodies and humanized antibodies, as well as ‘antibody fragments’ or ‘fragments thereof’ retaining the ability to specifically bind to the antigen (antigen-binding fragments) provided by any known technique, such as enzymatic cleavage, peptide synthesis, and recombinant techniques, and retaining the ability to be conjugated to a toxin. An antibody as generated can possess any isotype.

The term “immunoglobulin” as used herein is intended to refer to a class of structurally related glycoproteins consisting of two pairs of polypeptide chains, one pair of light (L) low molecular weight chains and one pair of heavy (H) chains, all four potentially inter-connected by disulfide bonds. The structure of immunoglobulins has been well characterized (see for instance Fundamental Immunology Ch. 7 (Paul, W., ed., 2nd ed. Raven Press, N.Y. (1989). Within the structure of the immunoglobulin, the two heavy chains are inter-connected via disulfide bonds in the so-called “hinge region”. Equally to the heavy chains each light chain is typically comprised of several regions; a light chain variable region (abbreviated herein as VL region) and a light chain constant region. Furthermore, the VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4. CDR sequences are defined according to IMGT (see Lefranc et al. (1999) and Brochet et al. (2008)).

The term “immunoglobulin heavy chain” or “heavy chain of an immunoglobulin” as used herein is intended to refer to one of the heavy chains of an immunoglobulin. A heavy chain is typically comprised of a heavy chain variable (abbreviated herein as VH) region and a heavy chain constant region (abbreviated herein as CH) which defines the isotype of the immunoglobulin. The heavy chain constant region typically is comprised of three domains, CH1, CH2, and CH3.

The term “immunoglobulin light chain” or “light chain of an immunoglobulin” as used herein is intended to refer to one of the light chains of an immunoglobulin. A light chain is typically comprised of a light chain variable (abbreviated herein as VL) region and a light chain constant region (abbreviated herein as CL). The light chain constant region typically is comprised of one domain, CL.

The terms “monoclonal antibody”, “monoclonal Ab”, “monoclonal antibody composition”, “mAb”, or the like, as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be produced by a hybridoma which includes a B cell obtained from a transgenic or transchromosomal non-human animal, such as a transgenic mouse, having a genome comprising a human heavy chain transgene and a light chain transgene, fused to an immortalized cell.

The term “full-length antibody” when used herein, refers to an antibody (e.g., a parent or variant antibody) which contains all heavy and light chain constant and variable domains corresponding to those that are normally found in a wild-type antibody of that isotype.

As used herein, “isotype” refers to the immunoglobulin class (for instance IgG1, IgG2, IgG3, IgG4, IgD, IgA, IgE, or IgM) that is encoded by heavy chain constant region genes.

The term “antigen-binding region” or “binding region” as used herein, refers to a region of an antibody which is capable of binding to the antigen. The antigen can be in solution, adhered to or bound to a surface or, e.g., present on a cell, bacterium, or virion. The terms “antigen” and “target” may, unless contradicted by the context, be used interchangeably in the context of the present invention.

The term “epitope” means a protein determinant capable of specific binding to an antibody. Epitopes usually consist of surface groupings of molecules such as amino acids, sugar side chains or a combination thereof and usually have specific three dimensional structural characteristics, as well as specific charge characteristics. Conformational and non conformational epitopes are distinguished in that the binding to the former but not the latter is lost in the presence of denaturing solvents. The epitope may comprise amino acid residues which are directly involved in the binding, and other amino acid residues, which are not directly involved in the binding, such as amino acid residues which are effectively blocked or covered by the specific antigen binding peptide (in other words, the amino acid residue is within the footprint of the specific antigen binding peptide).

The term “binding” as used herein refers to the binding of an antibody to a predetermined antigen or target, typically with a binding affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less when determined by for instance surface plasmon resonance (SPR) technology in a BIAcore 3000 instrument using the antigen as the ligand and the protein as the analyte, and binds to the predetermined antigen with an affinity corresponding to a K_{D} that is at least ten-fold lower, such as at least 100 fold lower, for instance at least 1,000 fold lower, such as at least 10,000 fold lower, for instance at least 100,000 fold lower than its affinity for binding to a non-specific antigen (e.g., BSA, casein) other than the predetermined antigen or a closely-related antigen. The amount with which the affinity is lower is dependent on the K_{D} of the protein, so that when the K_{D} of the protein is very low (that is, the protein is highly specific), then the amount with which the affinity for the antigen is lower than the affinity for a non-specific antigen may be at least 10,000 fold. The term “K_{D}” (M), as used herein, refers to the dissociation equilibrium constant of a particular antibody-antigen interaction, and is obtained by dividing k_{d} by kₐ.

The term “k_{d}” (sec⁻¹), as used herein, refers to the dissociation rate constant of a particular antibody-antigen interaction. Said value is also referred to as the k_{off} value.

The term “kₐ” (M⁻¹×sec⁻¹), as used herein, refers to the association rate constant of a particular antibody-antigen interaction.

The term “K_{D}” (M), as used herein, refers to the dissociation equilibrium constant of a particular antibody-antigen interaction.

The term “K_{A}” (M⁻¹), as used herein, refers to the association equilibrium constant of a particular antibody-antigen interaction and is obtained by dividing the kₐ by the k_{d}.

The terms “antibody binding AXL”, “AXL-antibody” or “anti-AXL antibody” as used herein, refers to any antibody binding an epitope on the extracellular part of AXL.

“Treatment” refers to the administration of an effective amount of a therapeutically active compound as described herein to a subject with the purpose of easing, ameliorating, arresting or eradicating (curing) symptoms or disease states of the subject.

As used herein, the term “subject” is typically a human to whom an antibody binding to AXL or an ADC comprising such antibody is administered, and who may benefit from the administration of the antibody binding to AXL or the ADC comprising such antibody, including for instance human patients diagnosed as having a cancer that may be treated by killing of AXL-expressing cells, directly or indirectly.

The term “isotype” as used herein refers to the immunoglobulin class (for instance IgG1, IgG2, IgG3, IgG4, IgD, IgA, IgE, or IgM) or any allotypes thereof, such as IgG1m(za) and IgG1m(f)) that is encoded by heavy chain constant region genes. Further, each heavy chain isotype can be combined with either a kappa (κ) or lambda (λ) light chain.

The term “full-length antibody” when used herein, refers to an antibody (e.g., a parent or variant antibody) which contains all heavy and light chain constant and variable domains corresponding to those that are normally found in a wild-type antibody of that isotype. A full-length antibody according to the present invention may be produced by a method comprising the steps of (i) cloning the CDR sequences into a suitable vector comprising complete heavy chain sequences and complete light chain sequence, and (ii) expressing the complete heavy and light chain sequences in suitable expression systems. It is within the knowledge of the skilled person to produce a full-length antibody when starting out from either CDR sequences or full variable region sequences. Thus, the skilled person would know how to generate a full-length antibody for use according to the present invention.

The percent identity between two nucleotide sequences may be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences may also be determined using the algorithm of E. Meyers and W. Miller, Comput. Appl. Biosci 4, 11-17 (1988)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444 453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The term “amino acid substitution” embraces a substitution into any one or the other nineteen natural amino acids, or into other amino acids, such as non-natural amino acids. For example, an amino acid may be substituted for another conservative or non-conservative amino acid. Amino acid residues may also be divided into classes defined by alternative physical and functional properties. Thus, classes of amino acids may be reflected in one or both of the following lists:

Amino acid residue of conservative class:

Acidic Residues: D and E

Basic Residues: K, R, and H

Hydrophilic Uncharged Residues: S, T, N, and Q

Aliphatic Uncharged Residues: G, A, V, L, and I

Non-polar Uncharged Residues: C, M, and P

Aromatic Residues: F, Y, and W

Alternative Physical and Functional Classifications of Amino Acid Residues:

Alcohol group-containing residues: S and T

Aliphatic residues: I, L, V, and M

Cycloalkenyl-associated residues: F, H, W, and Y

Hydrophobic residues: A, C, F, G, H, I, L, M, R, T, V, W, and Y

Negatively charged residues: D and E

Polar residues: C, D, E, H, K, N, Q, R, S, and T

Positively charged residues: H, K, and R

Small residues: A, C, D, G, N, P, S, T, and V

Very small residues: A, G, and S

Residues involved in turn formation: A, C, D, E, G, H, K, N, Q, R, S, P, and T

Flexible residues: Q, T, K, S, G, P, D, E, and R

The term “buffer” as used herein denotes a pharmaceutically acceptable buffer. The term “buffer” encompasses those agents which maintain the pH value of a solution, e.g., in an acceptable range and includes, but is not limited to, histidine, citrate, MES, phosphate, TRIS© (tris (hydroxymethyl)aminomethane), carbonic acid, succinate, glycolate and the like, as described herein. Generally, the “buffer” as used herein has a pKa and buffering capacity suitable for the pH range of about 5 to about 7, preferably of about 5.5 to 6.5, preferably about 5.8 to 6.2, such as about pH 6 or about pH 6.0.

The term “bulking agent” includes agents that can provide additional structure to a freeze-dried product (e.g., to provide a pharmaceutically acceptable cake). Commonly used bulking agents include mannitol, glycine, and the like. In addition to providing a pharmaceutically acceptable cake, bulking agents also typically impart useful qualities to the lyophilized composition such as modifying the collapse temperature, providing freeze-thaw protection, further enhancing the protein stability over long-term storage, and the like. These agents can also serve as tonicity modifiers.

The term “stabilizer” as used herein includes agents that provide stability to a protein, e.g., serving as a cryoprotectant during freezing and/or a lyoprotectant during a (freeze-) drying or ‘dehydration’ process. Suitable stabilizers include non-reducing sugars or saccharides and sugar alcohols such as sucrose, trehalose, mannitol, xylitol and the like, as well as amino acids such as glycine, alanine and lysine. Stabilizers can also serve as bulking agents, tonicity-modifying and/or viscosity-increasing agents.

A “surfactant” as used herein is a compound that is typically used in pharmaceutical formulations to prevent drug adsorption to surfaces and or aggregation. Furthermore, surfactants lower the surface tension (or interfacial tension) between two liquids or between a liquid and a solid. For example, an exemplary surfactant can significantly lower the surface tension when present at very low concentrations (e.g., 5% w/w or less, such as 3% w/w or less, such as 1% w/w or less). Surfactants are amphiphilic, which means they are usually composed of both hydrophilic and hydrophobic or lipophilic groups, thus being capable of forming micelles or similar self-assembled structures in aqueous solutions. Known surfactants for pharmaceutical use include glycerol monooleat, benzethonium chloride, sodium docusate, phospholipids, polyethylene alkyl ethers, sodium lauryl sulfate and tricaprylin (anionic surfactants); benzalkonium chloride, citrimide, cetylpyridinium chloride and phospholipids (cationic surfactants); and alpha tocopherol, glycerol monooleate, myristyl alcohol, phospholipids, poloxamers, polyoxyethylene alkyl ethers, polyoxyethylene castor oil derivatives, polyoxyethylene sorbintan fatty acid esters, polyoxyethylene sterarates, polyoxyl 15 hydroxystearate, polyoxylglycerides, polysorbates, propylene glycol dilaurate, propylene glycol monolaurate, sorbitan esters sucrose palmitate, sucrose stearate, tricaprylin and TPGS (Nonionic and zwitterionic surfactants).

A “diluent” of interest herein is one which is pharmaceutically acceptable (safe and non-toxic for administration to a human) and is useful for the preparation of a reconstituted formulation. Exemplary diluents are liquids, preferably aqueous, and include sterile water, bacteriostatic water for injection (BWFI), a pH buffered solution (e.g. phosphate-buffered saline), sterile saline solution, Ringer's solution or dextrose solution.

TABLE 1
SEQUENCES

| SEQ ID NO: | Name | Amino acid sequence | Comments |
|---|---|---|---|
| SEQ ID NO: 1 | Homo Sapiens AXL | MAWRCPRMGRVPLAWCLALCGWACMAPRGTQAEES | |
| | protein (Swissprot | PFVGNPGNITGARGLTGTLRCQLQVQGEPPEVHWLRD | |
| | P30530) | GQILELADSTQTQVPLGEDEQDDWIVVSQLRITSLQLSD | |
| | | TGQYQCLVFLGHQTFVSQPGYVGLEGLPYFLEEPEDRTV | |
| | | AANTPFNLSCQAQGPPEPVDLLWLQDAVPLATAPGHG | |
| | | PQRSLHVPGLNKTSSFSCEAHNAKGVTTSRTATITVLPQ | |
| | | QPRNLHLVSRQPTELEVAWTPGLSGIYPLTHCTLQAVLS | |
| | | DDGMGIQAGEPDPPEEPLTSQASVPPHQLRLGSLHPHT | |
| | | PYHIRVACTSSQGPSSWTHWLPVETPEGVPLGPPENISA | |
| | | TRNGSQAFVHWQEPRAPLQGTLLGYRLAYQGQDTPEV | |
| | | LMDIGLRQEVTLELQGDGSVSNLTVCVAAYTAAGDGP | |
| | | WSLPVPLEAWRPGQAQPVHQLVKEPSTPAFSWPWWY | |
| | | VLLGAVVAAACVLILALFLVHRRKKETRYGEVFEPTVERG | |
| | | ELVVRYRVRKSYSRRTTEATLNSLGISEELKEKLRDVMVD | |
| | | RHKVALGKTLGEGEFGAVMEGQLNQDDSILKVAVKTM | |
| | | KIAICTRSELEDFLSEAVCMKEFDHPNVMRLIGVCFQGS | |
| | | ERESFPAPVVILPFMKHGDLHSFLLYSRLGDQPVYLPTQ | |
| | | MLVKFMADIASGMEYLSTKRFIHRDLAARNCMLNENM | |
| | | SVCVADFGLSKKIYNGDYYRQGRIAKMPVKWIAIESLAD | |
| | | RVYTSKSDVWSFGVTMWEIATRGQTPYPGVENSEIYDY | |
| | | LRQGNRLKQPADCLDGLYALMSRCWELNPQDRPSFTE | |
| | | LREDLENTLKALPPAQEPDEILYVNMDEGGGYPEPPGA | |
| | | AGGADPPTQPDPKDSCSCLTAAEVHPAGRYVLCPSTTP | |
| | | SPAQPADRGSPAAPGQEDGA | |
| SEQ ID NO: 2 | Cynomolgus | AWRCPRMGRVPLAWCLALCGWVCMAPRGTQAEESP | |
| | monkey AXL | FVGNPGNITGARGLTGTLRCQLQVQGEPPEVHWLRDG | |
| | (GenBank number | QILELADSTQTQVPLGEDEQDDWIVVSQLRIASLQLSDA | |
| | HB387229.1) | GQYQCLVFLGHQNFVSQPGYVGLEGLPYFLEEPEDRIV | |
| | | AANTPFNLSCQAQGPPEPVDLLWLQDAVPLATAPGHG | |
| | | PQRNLHVPGLNKTSSFSCEAHNAKGVTTSRTATITVLPQ | |
| | | QPRNLHLVSRQPTELEVAWTPGLSGIYPLTHCTLQAVLS | |
| | | DDGMGIQAGEPDPPEEPLTLQASVPPHQLRLGSLHPHT | |
| | | PYHIRVACTSSQGPSSWTHWLPVETPEGVPLGPPENISA | |
| | | TRNGSQAFVHWQEPRAPLQGTLLGYRLAYQGQDTPEV | |
| | | LMDIGLRQEVTLELQGDGSVSNLTVCVAAYTAAGDGP | |
| | | WSLPVPLEAWRPGQAQPVHQLVKETSAPAFSWPWW | |
| | | YILLGAVVAAACVLILALFLVHRRKKETRYGEVFEPTVER | |
| | | GELVVRYRVRKSYSRRTTEATLNSLGISEELKEKLRDVMV | |
| | | DRHKVALGKTLGEGEFGAVMEGQLNQDDSILKVAVKT | |
| | | MKIAICTRSELEDFLSEAVCMKEFDHPNVMRLIGVCFQG | |
| | | SERESFPAPVVILPFMKHGDLHSFLLYSRLGDQPVYLPTQ | |
| | | MLVKFMADIASGMEYLSTKRFIHRDLAARNCMLNENM | |
| | | SVCVADFGLSKKIYNGDYYRQGRIAKMPVKWIAIESLAD | |
| | | RVYTSKSDVWSFGVTMWEIATRGQTPYPGVENSEIYDY | |
| | | LRQGNRLKQPADCLDGLYALMSRCWELNPQDRPSFTE | |
| | | LREDLENTLKALPPAQEPDEILYVNMDEGGGYPEPPGA | |
| | | AGGADPPTQLDPKDSCSCLTSAEVHPAGRYVLCPSTAPS | |
| | | PAQPADRGSPAAPGQEDGA | |
| SEQ ID NO: 3 | Homo Sapiens | MRPSGTAGAALLALLAALCPASRALEEKKVCQGTSNKLT | |
| | EGFR | QLGTFEDHFLSLQRMFNNCEVVLGNLEITYVQRNYDLSF | |
| | | LKTIQEVAGYVLIALNTVERIPLENLQIIRGNMYYENSYAL | |
| | | AVLSNYDANKTGLKELPMRNLQEILHGAVRFSNNPALC | |
| | | NVESIQWRDIVSSDFLSNMSMDFQNHLGSCQKCDPSC | |
| | | PNGSCWGAGEENCQKLTKIICAQQCSGRCRGKSPSDCC | |
| | | HNQCAAGCTGPRESDCLVCRKFRDEATCKDTCPPLMLY | |
| | | NPTTYQMDVNPEGKYSFGATCVKKCPRNYVVTDHGSC | |
| | | VRACGADSYEMEEDGVRKCKKCEGPCRKVCNGIGIGEF | |
| | | KDSLSINATNIKHFKNCTSISGDLHILPVAFRGDSFTHT | |
| | | PPLDPQELDILKTVKEITGFLLIQAWPENRTDLHAFENLEI | |
| | | IRGRTKQHGQFSLAVVSLNITSLGLRSLKEISDGDVIISGN | |
| | | KNLCYANTINWKKLFGTSGQKTKIISNRGENSCKATGQ | |
| | | VCHALCSPEGCWGPEPRDCVSCRNVSRGRECVDKCNLL | |
| | | EGEPREFVENSECIQCHPECLPQAMNITCTGRGPDNCI | |
| | | QCAHYIDGPHCVKTCPAGVMGENNTLVWKYADAGHV | |
| | | CHLCHPNCTYGCTGPGLEGCPTNGPKIPSIATGMVGALL | |
| | | LLLVVALGIGLFMRRRHIVRKRTLRRLLQERELVEPLTPSG | |
| | | EAPNQALLRILKETEFKKIKVLGSGAFGTVYKGLWIPEGE | |
| | | KVKIPVAIKELREATSPKANKEILDEAYVMASVDNPHVC | |
| | | RLLGICLTSTVQLITQLMPFGCLLDYVREHKDNIGSQYLL | |
| | | NWCVQIAKGMNYLEDRRLVHRDLAARNVLVKTPQHVK | |
| | | ITDFGLAKLLGAEEKEYHAEGGKVPIKWMALESILHRIYT | |
| | | QSDVWSYGVTVWELMTFGSKPYDGIPASEISSILEKGER | |
| | | LPQPPICTIDVYMIMVKCWMIDADSRPKFRELIIEFSKM | |
| | | ARDPQRYLVIQGDERMHLPSPTDSNFYRALMDEEDMD | |
| | | DVVDADEYLIPQQGFFSSPSTSRTPLLSSLSATSNNSTVA | |
| | | CIDRNGLQSCPIKEDSFLQRYSSDPTGALTEDSIDDTFLP | |
| | | VPEYINQSVPKRPAGSVQNPVYHNQPLNPAPSRDPHY | |
| | | QDPHSTAVGNPEYLNTVQPTCVNSTFDSPAHWAQKGS | |
| | | HQISLDNPDYQQDFFPKEAKPNGIFKGSTAENAEYLRVA | |
| | | PQSSEFIGA | |
| SEQ ID NO: 4 | Homo Sapiens ALK | MGAIGLLWLLPLLLSTAAVGSGMGTGQRAGSPAAGPP | |
| | tyrosine kinase | LQPREPLSYSRLQRKSLAVDFVVPSLFRVYARDLLLPPSSS | |
| | | ELKAGRPEARGSLALDCAPLLRLLGPAPGVSWTAGSPAP | |
| | | AEARTLSRVLKGGSVRKLRRAKQLVLELGEEAILEGCVGP | |
| | | PGEAAVGLLQFNLSELFSWWIRQGEGRLRIRLMPEKKA | |
| | | SEVGREGRLSAAIRASQPRLLFQIFGTGHSSLESPTNMPS | |
| | | PSPDYFTWNLTWIMKDSFPFLSHRSRYGLECSFDFPCEL | |
| | | EYSPPLHDLRNQSWSWRRIPSEEASQMDLLDGPGAERS | |
| | | KEMPRGSFLLLNTSADSKHTILSPWMRSSSEHCTLAVSV | |
| | | HRHLQPSGRYIAQLLPHNEAAREILLMPTPGKHGWTVL | |
| | | QGRIGRPDNPFRVALEYISSGNRSLSAVDFFALKNCSEGT | |
| | | SPGSKMALQSSFTCWNGTVLQLGQACDFHQDCAQGE | |
| | | DESQMCRKLPVGFYCNFEDGFCGWTQGTLSPHTPQW | |
| | | QVRTLKDARFQDHQDHALLLSTTDVPASESATVTSATFP | |
| | | APIKSSPCELRMSWLIRGVLRGNVSLVLVENKTGKEQGR | |
| | | MVWHVAAYEGLSLWQWMVLPLLDVSDRFWLQMVA | |
| | | WWGQGSRAIVAFDNISISLDCYLTISGEDKILQNTAPKSR | |
| | | NLFERNPNKELKPGENSPRQTPIFDPTVHWLFTTCGASG | |
| | | PHGPTQAQCNNAYQNSNLSVEVGSEGPLKGIQIWKVP | |
| | | ATDTYSISGYGAAGGKGGKNTMMRSHGVSVLGIFNLEK | |
| | | DDMLYILVGQQGEDACPSTNQLIQKVCIGENNVIEEEIR | |
| | | VNRSVHEWAGGGGGGGGATYVFKMKDGVPVPLIIAA | |
| | | GGGGRAYGAKTDTFHPERLENNSSVLGLNGNSGAAGG | |
| | | GGGWNDNTSLLWAGKSLQEGATGGHSCPQAMKKW | |
| | | GWETRGGFGGGGGGCSSGGGGGGYIGGNAASNNDP | |
| | | EMDGEDGVSFISPLGILYTPALKVMEGHGEVNIKHYLNC | |
| | | SHCEVDECHMDPESHKVICFCDHGTVLAEDGVSCIVSPT | |
| | | PEPHLPLSLILSVVTSALVAALVLAFSGIMIVYRRKHQELQ | |
| | | AMQMELQSPEYKLSKLRTSTIMTDYNPNYCFAGKTSSIS | |
| | | DLKEVPRKNITLIRGLGHGAFGEVYEGQVSGMPNDPSP | |
| | | LQVAVKTLPEVCSEQDELDFLMEALIISKFNHQNIVRCIG | |
| | | VSLQSLPRFILLELMAGGDLKSFLRETRPRPSQPSSLAML | |
| | | DLLHVARDIACGCQYLEENHFIHRDIAARNCLLTCPGPG | |
| | | RVAKIGDFGMARDIYRASYYRKGGCAMLPVKWMPPEA | |
| | | FMEGIFTSKTDTWSFGVLLWEIFSLGYMPYPSKSNQEV | |
| | | LEFVTSGGRMDPPKNCPGPVYRIMTQCWQHQPEDRP | |
| | | NFAIILERIEYCTQDPDVINTALPIEYGPLVEEEEKVPVRP | |
| | | KDPEGVPPLLVSQQAKREEERSPAAPPPLPTTSSGKAAKK | |
| | | PTAAEISVRVPRGPAVEGGHVNMAFSQSNPPSELHKVH | |
| | | GSRNKPTSLWNPTYGSWFTEKPTKKNNPIAKKEPHDRG | |
| | | NLGLEGSCTVPPNVATGRLPGASLLLEPSSLTANMKEVP | |
| | | LFRLRHFPCGNVNYGYQQQGLPLEAATAPGAGHYEDTI | |
| | | LKSKNSMNQPGP | |
| SEQ ID NO: 5 | Homo Sapiens | MDGFAGSLDDSISAASTSDVQDRLSALESRVQQQEDEI | |
| | EMAPL4 | TVLKAALADVLRRLAISEDHVASVKKSVSSKGQPSPRAVI | |
| | | PMSCITNGSGANRKPSHTSAVSIAGKETLSSAAKSGTEK | |
| | | KKEKPQGQREKKEESHSNDQSPQIRASPSPQPSSQPLQI | |
| | | HRQTPESKNATPTKSIKRPSPAEKSHNSWENSDDSRNKL | |
| | | SKIPSTPKLIPKVTKTADKHKDVIINQEGEYIKMFMRGRPI | |
| | | TMFIPSDVDNYDDIRTELPPEKLKLEWAYGYRGKDCRA | |
| | | NVYLLPTGKIVYFIASVVVLFNYEERTQRHYLGHTDCVKC | |
| | | LAIHPDKIRIATGQIAGVDKDGRPLQPHVRVWDSVTLST | |
| | | LQIIGLGTFERGVGCLDFSKADSGVHLCIIDDSNEHMLTV | |
| | | WDWQKKAKGAEIKTTNEVVLAVEFHPTDANTIITCGKS | |
| | | HIFFWTWSGNSLTRKQGIFGKYEKPKFVQCLAFLGNGD | |
| | | VLTGDSGGVMLIWSKTTVEPTPGKGPKGVYQISKQIKA | |
| | | HDGSVFTLCQMRNGMLLTGGGKDRKIILWDHDLNPER | |
| | | EIEVPDQYGTIRAVAEGKADQFLVGTSRNFILRGTFNDG | |
| | | FQIEVQGHTDELWGLATHPFKDLLLTCAQDRQVCLWN | |
| | | SMEHRLEWTRLVDEPGHCADFHPSGTVVAIGTHSGRW | |
| | | FVLDAETRDLVSIHTDGNEQLSVMRYSIDGTFLAVGSHD | |
| | | NFIYLYVVSENGRKYSRYGRCTGHSSYITHLDWSPDNKYI | |
| | | MSNSGDYEILYWDIPNGCKLIRNRSDCKDIDWTTYTCVL | |
| | | GFQVFGVWPEGSDGTDINALVRSHNRKVIAVADDFCK | |
| | | VHLFQYPCSKAKAPSHKYSAHSSHVTNVSFTHNDSHLIS | |
| | | TGGKDMSIIQWKLVEKLSLPQNETVADTTLTKAPVSSTE | |
| | | SVIQSNTPTPPPSQPLNETAEEESRISSSPTLLENSLEQTV | |
| | | EPSEDHSEEESEEGSGDLGEPLYEEPCNEISKEQAKATLL | |
| | | EDQQDPSPSS | |
| SEQ ID NO: 6 | Homo Sapiens | MADLAECNIKVMCRFRPLNESEVNRGDKYIAKFQGEDT | |
| | Kinesin-1 heavy | VVIASKPYAFDRVFQSSTSQEQVYNDCAKKIVKDVLEGY | |
| | chain (KINH) | NGTIFAYGQTSSGKTHTMEGKLHDPEGMGIIPRIVQDIF | |
| | | NYIYSMDENLEFHIKVSYFEIYLDKIRDLLDVSKTNLSVHE | |
| | | DKNRVPYVKGCTERFVCSPDEVMDTIDEGKSNRHVAVT | |
| | | NMNEHSSRSHSIFLINVKQENTQTEQKLSGKLYLVDLAG | |
| | | SEKVSKTGAEGAVLDEAKNINKSLSALGNVISALAEGSTY | |
| | | VPYRDSKMTRILQDSLGGNCRTTIVICCSPSSYNESETKS | |
| | | TLLFGQRAKTIKNTVCVNVELTAEQWKKKYEKEKEKNKI | |
| | | LRNTIQWLENELNRWRNGETVPIDEQFDKEKANLEAFT | |
| | | VDKDITLTNDKPATAIGVIGNFTDAERRKCEEEIAKLYKQ | |
| | | LDDKDEEINQQSQLVEKLKTQMLDQEELLASTRRDQDN | |
| | | MQAELNRLQAENDASKEEVKEVLQALEELAVNYDQKS | |
| | | QEVEDKTKEYELLSDELNQKSATLASIDAELQKLKEMTN | |
| | | HQKKRAAEMMASLLKDLAEIGIAVGNNDVKQPEGTG | |
| | | MIDEEFTVARLYISKMKSEVKTMVKRCKQLESTQTESNK | |
| | | KMEENEKELAACQLRISQHEAKIKSLTEYLQNVEQKKRQ | |
| | | LEESVDALSEELVQLRAQEKVHEMEKEHLNKVQTANEV | |
| | | KQAVEQQIQSHRETHQKQISSLRDEVEAKAKLITDLQDQ | |
| | | NQKMMLEQERLRVEHEKLKATDQEKSRKLHELTVMQD | |
| | | RREQARQDLKGLEETVAKELQTLHNLRKLFVQDLATRVK | |
| | | KSAEIDSDDTGGSAAQKQKISFLENNLEQLTKVHKQLVR | |
| | | DNADLRCELPKLEKRLRATAERVKALESALKEAKENASR | |
| | | DRKRYQQEVDRIKEAVRSKNMARRGHSAQIAKPIRPGQ | |
| | | HPAASPTHPSAIRGGGAFVQNSQPVAVRGGGG | |
| | | KQV | |
| SEQ ID NO: 7 | Homo Sapiens | MYDNMSTMVYIKEDKLEKLTQDEIISKTKQVIQGLEALK | |
| | Kinesin light | NEHNSILQSLLETLKCLKKDDESNLVEEKSNMIRKSLEML | |
| | chain 1 (KLC1) | ELGLSEAQVMMALSNHLNAVESEKQKLRAQVRRLCQE | |
| | | NQWLRDELANTQQKLQKSEQSVAQLEEEKKHLEFMNQ | |
| | | LKKYDDDISPSEDKDTDSTKEPLDDLFPNDEDDPGQGIQ | |
| | | QQHSSAAAAAQQGGYEIPARLRTLHNLVIQYASQGRYE | |
| | | VAVPLCKQALEDLEKTSGHDHPDVATMLNILALVYRDQ | |
| | | NKYKDAANLLNDALAIREKTLGKDHPAVAATLNNLAVLY | |
| | | GKRGKYKEAEPLCKRALEIREKVLGKDHPDVAKQLNNLA | |
| | | LLCQNQGKYEEVEYYYQRALEIYQTKLGPDDPNVAKTK | |
| | | NNLASCYLKQGKFKQAETLYKEILTRAHEREFGSVDDEN | |
| | | KPIWMHAEEREECKGKQKDGTSFGEYGGWYKACKVDS | |
| | | PTVTTTLKNLGALYRRQGKFEAAETLEEAAMRSRKQGL | |
| | | DNVHKQRVAEVLNDPENMEKRRSRESLNVDVVKYESG | |
| | | PDGGEEVSMSVEWNGGVSGRASFCGKRQQQQWPGR | |
| | | RHR | |
| SEQ ID NO: 8 | Homo Sapiens | MNGQLDLSGKLIIKAQLGEDIRRIPIHNEDITYDELVLM | |
| | Protein TFG | MQRVFRGKLLSNDEVTIKYKDEDGDLITIFDSSDLSFAIQ | |
| | | CSRILKLTLFVNGQPRPLESSQVKYLRRELIELRNKVNRLL | |
| | | DSLEPPGEPGPSTNIPENDTVDGREEKSASDSSGKQSTQ | |
| | | VMAASMSAFDPLKNQDEINKNVMSAFGLTDDQVSGP | |
| | | PSAPAEDRSGTPDSIASSSSAAHPPGVQPQQPPYTGAQ | |
| | | TQAGQIEGQMYQQYQQQAGYGAQQPQAPPQQPQQ | |
| | | YGIQYSASYSQQTGPQQPQQFQGYGQQPTSQAPAPAF | |
| | | SGQPQQLPAQPPQQYQASNYPAQTYTAQTSQPTNYTV | |
| | | APASQPGMAPSQPGAYQPRPGFTSLPGSTMTPPPSGP | |
| | | NPYARNRPPFGQGYTQPGPGYR | |
| SEQ ID NO: 9 | Homo Sapiens | MAAVLQQVLERTELNKLPKSVQNKLEKFLADQQSEIDG | |
| | Nucleoprotein | LKGRHEKFKVESEQQYFEIEKRLSHSQERLVNETRECQSL | |
| | TPR | RLELEKLNNQLKALTEKNKELEIAQDRNIAIQSQFTRTKE | |
| | | ELEAEKRDLIRTNERLSQELEYLTEDVKRLNEKLKESNTTK | |
| | | GELQLKLDELQASDVSVKYREKRLEQEKELLHSQNTWLN | |
| | | TELKTKTDELLALGREKGNEILELKCNLENKKEEVSRLEEQ | |
| | | MNGLKTSNEHLQKHVEDLLTKLKEAKEQQASMEEKFH | |
| | | NELNAHIKLSNLYKSAADDSEAKSNELTRAVEELHKLLKE | |
| | | AGEANKAIQDHLLEVEQSKDQMEKEMLEKIGRLEKELE | |
| | | NANDLLSATKRKGAILSEEELAAMSPTAAAVAKIVKPGM | |
| | | KLTELYNAYVETQDQLLLEKLENKRINKYLDEIVKEVEAK | |
| | | APILKRQREEYERAQKAVASLSVKLEQAMKEIQRLQEDT | |
| | | DKANKQSSVLERDNRRMEIQVKDLSQQIRVLLMELEEA | |
| | | RGNHVIRDEEVSSADISSSSEVISQHLVSYRNIEELQQQN | |
| | | QRLLVALRELGETREREEQETTSSKITELQLKLESALTELE | |
| | | QLRKSRQHQMQLVDSIVRQRDMYRILLSQTTGVAIPLH | |
| | | ASSLDDVSLASTPKRPSTSQTVSTPAPVPVIESTEAIEAKA | |
| | | ALKQLQEIFENYKKEKAENEKIQNEQLEKLQEQVTDLRS | |
| | | QNTKISTQLDFASKRYEMLQDNVEGYRREITSLHERNQK | |
| | | LTATTQKQEQIINTMTQDLRGANEKLAVAEVRAENLKK | |
| | | EKEMLKLSEVRLSQQRESLLAEQRGQNLLLTNLQTIQGIL | |
| | | ERSETETKQRLSSQIEKLEHEISHLKKKLENEVEQRHTLTR | |
| | | NLDVQLLDTKRQLDTETNLHLNTKELLKNAQKEIATLKQ | |
| | | HLSNMEVQVASQSSQRTGKGQPSNKEDVDDLVSQLRQ | |
| | | TEEQVNDLKERLKTSTSNVEQYQAMVTSLEESLNKEKQ | |
| | | VTEEVRKNIEVRLKESAEFQTQLEKKLMEVEKEKQELQD | |
| | | DKRRAIESMEQQLSELKKTLSSVQNEVQEALQRASTALS | |
| | | NEQQARRDCQEQAKIAVEAQNKYERELMLHAADVEAL | |
| | | QAAKEQVSKMASVRQHLEETTQKAESQLLECKASWEE | |
| | | RERMLKDEVSKCVCRCEDLEKQNRLLHDQIEKLSDKVVA | |
| | | SVKEGVQGPLNVSLSEEGKSQEQILEILRFIRREKEIAETR | |
| | | FEVAQVESLRYRQRVELLERELQELQDSLNAEREKVQVT | |
| | | AKTMAQHEELMKKTETMNVVMETNKMLREEKERLEQ | |
| | | DLQQMQAKVRKLELDILPLQEANAELSEKSGMLQAEKK | |
| | | LLEEDVKRWKARNQHLVSQQKDPDTEEYRKLLSEKEVH | |
| | | TKRIQQLTEEIGRLKAEIARSNASLTNNQNLIQSLKEDLN | |
| | | KVRTEKETIQKDLDAKIIDIQEKVKTITQVKKIGRRYKTQY | |
| | | EELKAQQDKVMETSAQSSGDHQEQHVSVQEMQELKE | |
| | | TLNQAETKSKSLESQVENLQKTLSEKETEARNLQEQTVQ | |
| | | LQSELSRLRQDLQDRTTQEEQLRQQITEKEEKTRKAIVA | |
| | | AKSKIAHLAGVKDQLTKENEELKQRNGALDQQKDELDV | |
| | | RITALKSQYEGRISRLERELREHQERHLEQRDEPQEPSNK | |
| | | VPEQQRQITLKTTPASGERGIASTSDPPTANIKPTPVVST | |
| | | PSKVTAAAMAGNKSTPRASIRPMVTPATVTNPTTTPTA | |
| | | TVMPTTQVESQEAMQSEGPVEHVPVFGSTSGSVRSTS | |
| | | PNVQPSISQPILTVQQQTQATAFVQPTQQSHPQIEPAN | |
| | | QELSSNIVEVVQSSPVERPSTSTAVFGTVSATPSSSLPKR | |
| | | TREEEEDSTIEASDQVSDDTVEMPLPKKLKSVTPVGTEEE | |
| | | VMAEESTDGEVETQVYNQDSQDSIGEGVTQGDYTPME | |
| | | DSEETSQSLQIDLGPLQSDQQTTTSSQDGQGKGDDVIVI | |
| | | DSDDEEEDDDENDGEHEDYEEDEEDDDDDEDDTGMG | |
| | | DEGEDSNEGTGSADGNDGYEADDAEGGDGTDPGTET | |
| | | EESMGGGEGNHRAADSQNSGEGNTGAAESSFSQEVSR | |
| | | EQQPSSASERQAPRAPQSPRRPPHPLPPRLTIHAPPQEL | |
| | | GPPVQRIQMTRRQSVGRGLQLTPGIGGMQQHFFDDE | |
| | | DRTVPSTPTLVVPHRTDGFAEAIHSPQVAGVPRFRFGPP | |
| | | EDMPQTSSSHSDLGQLASQGGLGMYETPLFLAHEEESG | |
| | | GRSVPTTPLQVAAPVTVFTESTTSDASEHASQSVPMVT | |
| | | TSTGTLSTTNETATGDDGDEVFVEAESEGISSEAGLEIDS | |
| | | QQEEEPVQASDESDLPSTSQDPPSSSSVDTSSSQPKPFR | |
| | | RVRLQTTLRQGVRGRQFNRQRGVSHAMGGRGGINRG | |
| | | NIN | |
| SEQ ID NO: 10 | Homo Sapiens | MDRMASSMKQVPNPLPKVLSRRGVGAGLEAAERESFE | |
| | Huntington- | RTQTVSINKAINTQEVAVKEKHARTCILGTHHEKGAQTF | |
| | interacting | WSVVNRLPLSSNAVLCWKFCHVFHKLLRDGHPNVLKDS | |
| | protein 1 (HIP-1) | LRYRNELSDMSRMWGHLSEGYGQLCSIYLKLLRTKMEY | |
| | | HTKNPRFPGNLQMSDRQLDEAGESDVNNFFQLTVEMF | |
| | | DYLECELNLFQTVFNSLDMSRSVSVTAAGQCRLAPLIQV | |
| | | ILDCSHLYDYTVKLLFKLHSCLPADTLQGHRDRFMEQFT | |
| | | KLKDLFYRSSNLQYFKRLIQIPQLPENPPNFLRASALSEHI | |
| | | SPVVVIPAEASSPDSEPVLEKDDLMDMDASQQNLFDNK | |
| | | FDDIFGSSFSSDPFNFNSQNGVNKDEKDHLIERLYREISG | |
| | | LKAQLENMKTESQRVVLQLKGHVSELEADLAEQQHLR | |
| | | QQAADDCEFLRAELDELRRQREDTEKAQRSLSEIERKAQ | |
| | | ANEQRYSKLKEKYSELVQNHADLLRKNAEVTKQVSMAR | |
| | | QAQVDLEREKKELEDSLERISDQGQRKTQEQLEVLESLK | |
| | | QELATSQRELQVLQGSLETSAQSEANWAAEFAELEKER | |
| | | DSLVSGAAHREEELSALRKELQDTQLKLASTEESMCQLA | |
| | | KDQRKMLLVGSRKAAEQVIQDALNQLEEPPLISCAGSA | |
| | | DHLLSTVTSISSCIEQLEKSWSQYLACPEDISGLLHSITLL | |
| | | AHLTSDAIAHGATTCLRAPPEPADSLTEACKQYGRETLAYL | |
| | | ASLEEEGSLENADSTAMRNCLSKIKAIGEELLPRGLDIKQ | |
| | | EELGDLVDKEMAATSAAIETATARIEEMLSKSRAGDTGV | |
| | | KLEVNERILGCCTSLMQAIQVLIVASKDLQREIVESGRGT | |
| | | ASPKEFYAKNSRWTEGLISASKAVGWGATVMVDAADL | |
| | | VVQGRGKFEELMVCSHEIAASTAQLVAASKVKADKDSP | |
| | | NLAQLQQASRGVNQATAGVVASTISGKSQIEETDNMD | |
| | | FSSMTLTQIKRQEMDSQVRVLELENELQKERQKLGELRK | |
| | | KHYELAGVAEGWEEGTEASPPTLQEVVTEKE | |
| SEQ ID NO: 11 | Homo Sapiens | MDEQAGPGVFFSNNHPGAGGAKGLGPLAEAAAAGDG | |
| | Striatin | AAAAGAARAQYSLPGILHFLQHEWARFEVERAQWEVE | |
| | | RAELQAQIAFLQGERKGQENLKKIDLVRRIKMLEYALKQE | |
| | | RAKYHKLKYGTELNQGDMKPPSYDSDEGNETEVQPQQ | |
| | | NSQLMWKQGRQLLRQYLQEVGYTDTILDVKSKRVRALL | |
| | | GFSSDVTDREDDKNQDSVVNGTEAEVKETAMIAKSELT | |
| | | DSASVLDNFKFLESAAADFSDEDEDDDVDGREKSVIDTS | |
| | | TIVRKKALPDSGEDRDTKEALKEFDFLVTSEEGDNESRSA | |
| | | GDGTDWEKEDQCLMPEAWNVDQGVITKLKEQYKKER | |
| | | KGKKGVKRPNRSKLQDMLANLRDVDELPSLQPSVGSPS | |
| | | RPSSSRLPEHEINRADEVEALTFPPSSGKSFIMGADEALE | |
| | | SELGLGELAGLTVANEADSLTYDIANNKDALRKTWNPKF | |
| | | TLRSHFDGIRALAFHPIEPVLITASEDHTLKMWNLQKTA | |
| | | PAKKSTSLDVEPIYTFRAHKGPVLCVVMSSNGEQCYSGG | |
| | | TDGLIQGWNTTNPNIDPYDSYDPSVLRGPLLGHTDAV | |
| | | WGLAYSAAHQRLLSCSADGTLRLWNTTEVAPALSVFND | |
| | | TKELGIPASVDLVSSDPSHMVASFSKGYTSIFNMETQQR | |
| | | ILTLESNVDTTANSSCQINRVISHPTLPISITAHEDRHIKF | |
| | | YDNNTGKLIHSMVAHLEAVTSLAVDPNGLYLMSGSHDCS | |
| | | IRLWNLESKTCIQEFTAHRKKFEESIHDVAFHPSKCYIASA | |
| | | GADALAKVFV | |
| SEQ ID NO: 12 | Homo Sapiens | MAQSKRHVYSRTPSGSRMSAEASARPLRVGSRVEVIGK | |
| | dynactin subunit | GHRGTVAYVGATLFATGKWVGVILDEAKGKNDGTVQG | |
| | 1 | RKYFTCDEGHGIFVRQSQIQVFEDGADTTSPETPDSSAS | |
| | | KVLKREGTDTTAKTSKLRGLKPKKAPTARKTTTRRPKPTR | |
| | | PASTGVAGASSSLGPSGSASAGELSSSEPSTPAQTPLAAP | |
| | | IIPTPVLTSPGAVPPLPSPSKEEEGLRAQVRDLEEKLETLR | |
| | | LKRAEDKAKLKELEKHKIQLEQVQEWKSKMQEQQADL | |
| | | QRRLKEARKEAKEALEAKERYMEEMADTADAIEMATLD | |
| | | KEMAEERAESLQQEVEALKERVDELTTDLEILKAEIEEKG | |
| | | SDGAASSYQLKQLEEQNARLKDALVRMRDLSSSEKQEH | |
| | | VKLQKLMEKKNQELEVVRQQRERLQEELSQAESTIDELK | |
| | | EQVDAALGAEEMVEMLTDRNLNLEEKVRELRETVGDLE | |
| | | AMNEMNDELQENARETELELREQLDMAGARVREAQK | |
| | | RVEAAQETVADYQQTIKKYRQLTAHLQDVNRELTNQQ | |
| | | EASVERQQQPPPETFDFKIKFAETKAHAKAIEMELRQM | |
| | | EVAQANRHMSLLTAFMPDSFLRPGGDHDCVLVLLLMP | |
| | | RLICKAELIRKQAQEKFELSENCSERPGLRGAAGEQLSFA | |
| | | AGLVYSLSLLQATLHRYEHALSQCSVDVYKKVGSLYPEM | |
| | | SAHERSLDFLIELLHKDQLDETVNVEPLTKAIKYYQHLYSI | |
| | | HLAEQPEDCTMQLADHIKFTQSALDCMSVEVGRLRAFL | |
| | | QGGQEATDIALLLRDLETSCSDIRQFCKKIRRRMPGTDA | |
| | | PGIPAALAFGPQVSDTLLDCRKHLTWVVAVLQEVAAAA | |
| | | AQLIAPLAENEGLLVAALEELAFKASEQIYGTPSSSPYECL | |
| | | RQSCNILISTMNKLATAMQEGEYDAERPPSKPPPVELRA | |
| | | AALRAEITDAEGLGLKLEDRETVIKELKKSLKIKGEELSEA | |
| | | NVRLSLLEKKLDSAAKDADERIEKVQTRLEETQALLRKKE | |
| | | KEFEETMDALQADIDQLEAEKAELKQRLNSQSKRTIEGL | |
| | | RGPPPSGIATLVSGIAGEEQQRGAIPGQAPGSVPGPGLV | |
| | | KDSPLLLQQISAMRLHISQLQHENSILKGAQMKASLASL | |
| | | PPLHVAKLSHEGPGSELPAGALYRKTSQLLETLNQLSTHT | |
| | | HVVDITRTSPAAKSPSAQLMEQVAQLKSLSDTVEKLKDE | |
| | | VLKETVSQRPGATVPTDFATFPSSAFLRAKEEQQDDTVY | |
| | | MGKVTFSCAAGFGQRHRLVLTQEQLHQLHSRLIS | |
| SEQ ID NO: 13 | Homo Sapiens | MASLTVKAYLLGKEDAAREIRRFSFCCSPEPEAEAEAAA | |
| | sequestosome-1 | GPGPCERLLSRVAALFPALRPGGFQAHYRDEDGDLVAF | |
| | | SSDEELTMAMSYVKDDIFRIYIKEKKECRRDHRPPCAQE | |
| | | APRNMVHPNVICDGCNGPVVGTRYKCSVCPDYDLCSV | |
| | | CEGKGLHRGHTKLAFPSPFGHLSEGFSHSRWLRKVKHG | |
| | | HFGWPGWEMGPPGNWSPRPPRAGEARPGPTAESAS | |
| | | GPSEDPSVNFLKNVGESVAAALSPLGIEVDIDVEHGGKR | |
| | | SRLTPVSPESSSTEEKSSSQPSSCCSDPSKPGGNVEGATQ | |
| | | SLAEQMRKIALESEGRPEEQMESDNCSGGDDDWTHLS | |
| | | SKEVDPSTGELQSLQMPESEGPSSLDPSQEGPTGLKEAA | |
| | | LYPHLPPEADPRLIESLSQMLSMGFSDEGGWLTRLLQTK | |
| | | NYDIGAALDTIQYSKHPPPL | |
| SEQ ID NO: 14 | Homo Sapiens | MEDSMDMDMSPLRPQNYLFGCELKADKDYHFKVDND | |
| | nucleophosmin | ENEHQLSLRTVSLGAGAKDELHIVEAEAMNYEGSPIKVT | |
| | | LATLKMSVQPTVSLGGFEITPPVVLRLKCGSGPVHISGQ | |
| | | HLVAVEEDAESEDEEEEDVKLLSISGKRSAPGGGSKVPQ | |
| | | KKVKLAADEDDDDDDEEDDDEDDDDDDFDDEEAEEKA | |
| | | PVKKSIRDTPAKNAQKSNQNGKDSKPSSTPRSKGQESFK | |
| | | KQEKTPKTPKGPSSVEDIKAKMQASIEKGGSLPKVEAKFI | |
| | | NYVKNCFRMTDQEAIQDLWQWRKSL | |
| SEQ ID NO: 15 | Homo Sapiens | MSRRKQGKPQHLSKREFSPEPLEAILTDDEPDHGPLGAP | |
| | B-cell | EGDHDLLTCGQCQMNFPLGDILIFIEHKRKQCNGSLCLE | |
| | lymphoma/ | KAVDKPPSPSPIEMKKASNPVEVGIQVTPEDDDCLSTSS | |
| | leukemia 11A | RGICPKQEHIADKLLHWRGLSSPRSAHGALIPTPGMSAE | |
| | | YAPQGICKDEPSSYTCTTCKQPFTSAWFLLQHAQNTHG | |
| | | LRIYLESEHGSPLTPRVGIPSGLGAECPSQPPLHGIHIADN | |
| | | NPFNLLRIPGSVSREASGLAEGRFPPTPPLFSPPPRHHLD | |
| | | PHRIERLGAEEMALATHHPSAFDRVLRLNPMAMEPPA | |
| | | MDFSRRLRELAGNTSSPPLSPGRPSPMQRLLQPFQPGS | |
| | | KPPFLATPPLPPLQSAPPPSQPPVKSKSCEFCGKTFKFQS | |
| | | NLVVHRRSHTGEKPYKCNLCDHACTQASKLKRHMKTH | |
| | | MHKSSPMTVKSDDGLSTASSPEPGTSDLVGSASSALKSV | |
| | | VAKFKSENDPNLIPENGDEEEEEDDEEEEEEEEEEEEELT | |
| | | ESERVDYGFGLSLEAARHHENSSRGAVVGVGDESRALP | |
| | | DVMQGMVLSSMQHFSEAFHQVLGEKHKRGHLAEAEG | |
| | | HRDTCDEDSVAGESDRIDDGTVNGRGCSPGESASGGLS | |
| | | KKLLLGSPSSLSPFSKRIKLEKEFDLPPAAMPNTENVYSQ | |
| | | WLAGYAASRQLKDPFLSFGDSRQSPFASSSEHSSENGSL | |
| | | RFSTPPGELDGGISGRSGTGSGGSTPHISGPGPGRPSSK | |
| | | EGRRSDTCEYCGKVFKNCSNLTVHRRSHTGERPYKCELC | |
| | | NYACAQSSKLTRHMKTHGQVGKDVYKCEICKMPFSVYS | |
| | | TLEKHMKKWHSDRVLNNDIKTE | |
| SEQ ID NO: 16 | Homo Sapiens | MVTGGGAAPPGTVTEPLPSVIVLSAGRKMAAAAAAAS | |
| | baculovirai IAP | GPGCSSAAGAGAAGVSEWLVLRDGCMHCDADGLHSL | |
| | repeat-containing | SYHPALNAILAVTSRGTIKVIDGTSGATLQASALSAKPGG | |
| | protein | QVKCQYISAVDKVIFVDDYAVGCRKDLNGILLLDTALQT | |
| | | PVSKQDDVVQLELPVTEAQQLLSACLEKVDISSTEGYDLF | |
| | | ITQLKDGLKNTSHETAANHKVAKWATVTFHLPHHVLKSI | |
| | | ASAIVNELKKINQNVAALPVASSVMDRLSYLLPSARPEL | |
| | | GVGPGRSVDRSLMYSEANRRETFTSWPHVGYRWAQP | |
| | | DPMAQAGFYHQPASSGDDRAMCFTCSVCLVCWEPTD | |
| | | EPWSEHERHSPNCPFVKGEHTQNVPLSVTLATSPAQFP | |
| | | CTDGTDRISCFGSGSCPHFLAAATKRGKICIWDVSKLMK | |
| | | VHLKFEINAYDPAIVQQLILSGDPSSGVDSRRPTLAWLE | |
| | | DSSSCSDIPKLEGDSDDLLEDSDSEEHSRSDSVTGHTSQK | |
| | | EAMEVSLDITALSILQQPEKLQWEIVANVLEDTVKDLEEL | |
| | | GANPCLTNSKSEKTKEKHQEQHNIPFPCLLAGGLLTYKS | |
| | | PATSPISSNSHRSLDGLSRTQGESISEQGSTDNESCTNSE | |
| | | LNSPLVRRTLPVLLLYSIKESDEKAGKIFSQMNNIMSKSL | |
| | | HDDGFTVPQIIEMELDSQEQLLLQDPPVTYIQQFADAA | |
| | | ANLTSPDSEKWNSVFPKPGTLVQCLRLPKFAEEENLCID | |
| | | SITPCADGIHLLVGLRTCPVESLSAINQVEALNNLNKLNS | |
| | | ALCNRRKGELESNLAVVNGANISVIQHESPADVQTPLIIQ | |
| | | PEQRNVSGGYLVLYKMNYATRIVTLEEEPIKIQHIKDPQD | |
| | | TITSLILLPPDILDNREDDCEEPIEDMQLTSKNGFEREKTS | |
| | | DISTLGHLVITTQGGYVKILDLSNFEILAKVEPPKKEGTEE | |
| | | QDTFVSVIYCSGTDRLCACTKGGELHFLQIGGTCDDIDE | |
| | | ADILVDGSLSKGIEPSSEGSKPLSNPSSPGISGVDLLVDQP | |
| | | FTLEILTSLVELTRFETLTPRFSATVPPCWVEVQQEQQQR | |
| | | RHPQHLHQQHHGDAAQHTRTWKLQTDSNSWDEHVF | |
| | | ELVLPKACMVGHVDFKFVLNSNITNIPQIQVTLLKNKAP | |
| | | GLGKVNALNIEVEQNGKPSLVDLNEEMQHMDVEESQC | |
| | | LRLCPFLEDHKEDILCGPVWLASGLDLSGHAGMLTLTSP | |
| | | KLVKGMAGGKYRSFLIHVKAVNERGTEEICNGGMRPVV | |
| | | RLPSLKHQSNKGYSLASLLAKVAAGKEKSSNVKNENTSG | |
| | | TRKSENLRGCDLLQEVSVTIRRFKKTSISKERVQRCAMLQ | |
| | | FSEFHEKLVNTLCRKTDDGQITEHAQSLVLDTLCWLAGV | |
| | | HSNGPGSSKEGNENLLSKTRKFLSDIVRVCFFEAGRSIAH | |
| | | KCARFLALCISNGKCDPCQPAFGPVLLKALLDNMSFLPA | |
| | | ATTGGSVYWYFVLLNYVKDEDLAGCSTACASLLTAVSR | |
| | | QLQDRLTPMEALLQTRYGLYSSPFDPVLFDLEMSGSSCK | |
| | | NVYNSSIGVQSDEIDLSDVLSGNGKVSSCTAAEGSFTSLT | |
| | | GLLEVEPLHFTCVSTSDGTRIERDDAMSSFGVTPAVGGL | |
| | | SSGTVGEASTALSSAAQVALQSLSHAMASAEQQLQVLQ | |
| | | EKQQQLLKLQQQKAKLEAKLHQTTAAAAAAASAVGPV | |
| | | HNSVPSNPVAAPGFFIHPSDVIPPTPKTTPLFMTPPLTPP | |
| | | NEAVSVVINAELAQLFPGSVIDPPAVNLAAHNKNSNKS | |
| | | RMNPLGSGLALAISHASHFLQPPPHQSIIIERMHSGARR | |
| | | FVTLDFGRPILLTDVLIPTCGDLASLSIDIWTLGEEVDGRR | |
| | | LVVATDISTHSLILHDLIPPPVCRFMKITVIGRYGSTNARA | |
| | | KIPLGFYYGHTYILPWESELKLMHDPLKGEGESANQPEI | |
| | | DQHLAMMVALQEDIQCRYNLACHRLETLLQSIDLPPLN | |
| | | SANNAQYFLRKPDKAVEEDSRVFSAYQDCIQLQLQLNL | |
| | | AHNAVQRLKVALGASRKMLSETSNPEDLIQTSSTEQLRT | |
| | | IIRYLLDTLLSLLHASNGHSVPAVLQSTFHAQACEELFKHL | |
| | | CISGTPKIRLHTGLLLVQLCGGERWWGQFLSNVLQELYN | |
| | | SEQLLIFPQDRVFMLLSCIGQRSLSNSGVLESLLNLLDNLL | |
| | | SPLQPQLPMHRRTEGVLDIPMISWVVMLVSRLLDYVAT | |
| | | VEDEAAAAKKPLNGNQWSFINNNLHTQSLNRSSKGSSS | |
| | | LDRLYSRKIRKQLVHHKQQLNLLKAKQKALVEQMEKEKI | |
| | | QSNKGSSYKLLVEQAKLKQATSKHFKDLIRLRRTAEWSR | |
| | | SNLDTEVTTAKESPEIEPLPFTLAHERCISVVQKLVLFLLS | |
| | | MDFTCHADLLLFVCKVLARIANATRPTIHLCEIVNEPQLE | |
| | | RLLLLLVGTDFNRGDISWGGAWAQYSLTCMLQDILAGE | |
| | | LLAPVAAEAMEEGTVGDDVGATAGDSDDSLQQSSVQL | |
| | | LETIDEPLTHDITGAPPLSSLEKDKEIDLELLQDLMEVDID | |
| | | PLDIDLEKDPLAAKVFKPISSTWYDYWGADYGTYNYNPY | |
| | | IGGLGIPVAKPPANTEKNGSQTVSVSVSQALDARLEVGL | |
| | | EQQAELMLKMMSTLEADSILQALTNTSPTLSQSPTGTD | |
| | | DSLLGGLQAANQTSQLIIQLSSVPMLNVCFNKLFSMLQV | |
| | | HHVQLESLLQLWLTLSLNSSSTGNKENGADIFLYNANRI | |
| | | PVISLNQASITSFLTVLAWYPNTLLRTWCLVLHSLTLMTN | |
| | | MQLNSGSSSAIGTQESTAHLLVSDPNLIHVLVKFLSGTSP | |
| | | HGTNQHSPQVGPTATQAMQEFLTRLQVHLSSTCPQIFS | |
| | | EFLLKLIHILSTERGAFQTGQGPLDAQVKLLEFTLEQNFE | |
| | | VVSVSTISAVIESVTFLVHHYITCSDKVMSRSGSDSSVGA | |
| | | RACFGGLFANLIRPGDAKAVCGEMTRDQLMFDLLKLVN | |
| | | ILVQLPLSGNREYSARVSVTTNTTDSVSDEEKVSGGKDG | |
| | | NGSSTSVQGSPAYVADLVLANQQIMSQILSALGLCNSS | |
| | | AMAMIIGASGLHLTKHENFHGGLDAISVGDGLFTILTTL | |
| | | SKKASTVHMMLQPILTYMACGYMGRQGSLATCQLSEP | |
| | | LLWFILRVLDTSDALKAFHDMGGVQLICNNMVTSTRAI | |
| | | VNTARSMVSTIMKFLDSGPNKAVDSTLKTRILASEPDNA | |
| | | EGIHNFAPLGTITSSSPTAQPAEVLLQATPPHRRARSAA | |
| | | WSYIFLPEEAWCDLTIHLPAAVLLKEIHIQPHLASLATCPS | |
| | | SVSVEVSADGVNMLPLSTPVVTSGLTYIKIQLVKAEVASA | |
| | | VCLRLHRPRDASTLGLSQIKLLGLTAFGTTSSATVNNPFL | |
| | | PSEDQVSKTSIGWLRLLHHCLTHISDLEGMMASAAAPT | |
| | | ANLLQTCAALLMSPYCGMHSPNIEVVLVKIGLQSTRIGL | |
| | | KLIDILLRNCAASGSDPTDLNSPLLFGRLNGLSSDSTIDIL | |
| | | YQLGTTQDPGTKDRIQALLKWVSDSARVAAMKRSGRM | |
| | | NYMCPNSSTVEYGLLMPSPSHLHCVAAILWHSYELLVEY | |
| | | DLPALLDQELFELLFNWSMSLPCNMVLKKAVDSLLCSM | |
| | | CHVHPNYFSLLMGWMGITPPPVQCHHRLSMTDDSKK | |
| | | QDLSSSLTDDSKNAQAPLALTESHLATLASSSQSPEAIKQ | |
| | | LLDSGLPSLLVRSLASFCFSHISSSESIAQSIDISQDKLRR | |
| | | HHVPQQCNKMPITADLVAPILRFLTEVGNSHIMKDWLG | |
| | | GSEVNPLWTALLFLLCHSGSTSGSHNLGAQQTSARSASL | |
| | | SSAATTGLTTQQRTAIENATVAFFLQCISCHPNNQKL | |
| | | MAQVLCELFQTSPQRGNLPTSGNISGFIRRLFLQLMLED | |
| | | EKVTMFLQSPCPLYKGRINATSHVIQHPMYGAGHKFRT | |
| | | LHLPVSTTLSDVLDRVSDTPSITAKLISEQKDDKEKKNHE | |
| | | EKEKVKAENGFQDNYSVVVASGLKSQSKRAVSATPPRP | |
| | | PSRRGRTIPDKIGSTSGAEAANKIIIVPVFHLFHKLLAGQ | |
| | | PLPAEMTLAQLLTLLYDRKLPQGYRSIDLTVKLGSRVITD | |
| | | PSLSKTDSYKRLHPEKDHGDLLASCPEDEALTPGDECMD | |
| | | GILDESLLETCPIQSPLQVFAGMGGLALIAERLPMLYPEVI | |
| | | QQVSAPVVTSTTQEKPKDSDQFEWVTIEQSGELVYEAP | |
| | | ETVAAEPPPIKSAVQTMSPIPAHSLAAFGLFLRLPGYAEV | |
| | | LLKERKHAQCLLRLVLGVTDDGEGSHILQSPSANVLPTLP | |
| | | FHVLRSLFSTTPLTTDDGVLLRRMALEIGALHLILVCLSAL | |
| | | SHHSPRVPNSSVNQTEPQVSSSHNPTSTEEQQLYWAK | |
| | | GTGFGTGSTASGWDVEQALTKQRLEEEHVTCLLQVLAS | |
| | | YINPVSSAVNGEAQSSHETRGQNSNALPSVLLELLSQSC | |
| | | LIPAMSSYLRNDSVLDMARHVPLYRALLELLRAIASCAA | |
| | | MVPLLLPLSTENGEEEEEQSECQTSVGTLLAKMKTCVDT | |
| | | YTNRLRSKRENVKTGVKPDASDQEPEGLTLLVPDIQKTA | |
| | | EIVYAATTSLRQANQEKKLGEYSKKAAMKPKPLSVLKSLE | |
| | | EKYVAVMKKLQFDTFEMVSEDEDGKLGFKVNYHYMSQ | |
| | | VKNANDANSAARARRLAQEAVTLSTSLPLSSSSSVFVRC | |
| | | DEERLDIMKVLITGPADTPYANGCFEFDVYFPQDYPSSP | |
| | | PLVNLETTGGHSVRFNPNLYNDGKVCLSILNTWHGRPE | |
| | | EKWNPQTSSFLQVLVSVQSLILVAEPYFNEPGYERSRGT | |
| | | PSGTQSSREYDGNIRQATVKWAMLEQIRNPSPCFKEVI | |
| | | HKHFYLKRVEIMAQCEEWIADIQQYSSDKRVGRTMSH | |
| | | HAAALKRHTAQLREELLKLPCPEGLDPDTDDAPEVCRAT | |
| | | TGAEETLMHDQVKPSSSKELPSDFQL | |
| SEQ ID NO: 17 | 107 VH | EVQLLESGGGLVQPGGSLRLSCAASGFTFSSYAMNWVR | HCo12-BalbC |
| | | QAPGKGLEWVSTTSGSGASTYYADSVKGRFTISRDNSK | Ig1 domain |
| | | NTLYLQMNSLRAEDTAVYYCAKIWIAFDIWGQGTMVT | binding Ab |
| | | VSS | |
| SEQ ID NO: 18 | 107 VL | EIVLTQSPGTLSLSPGERATLSCRASQSVSSSYLAWYQQK | |
| | | PGQAPRLLIYGASSRATGIPDRFSGSGSGTDFTLTISRLEP | |
| | | EDFAVYYCQQYGSSPYTFGQGTKLEIK | |
| SEQ ID NO: 19 | 107 VH CDR1 | GFTFSSYA | |
| SEQ ID NO: 20 | 107 VH CDR2 | TSGSGAST | |
| SEQ ID NO: 21 | 107 VH CDR3 | AKIWIAFDI | |
| SEQ ID NO: 22 | 107 VL CDR1 | QSVSSSY | |
| | 107 VL CDR2 | GAS | |
| SEQ ID NO: 23 | 107 VL CDR3 | QQYGSSPYT | |

### Specific Aspects and Embodiments of the Invention

In a first aspect the present invention provides a conjugate of monomethyl auristatin or a functional analog or derivative thereof and an antibody or antigen-binding fragment thereof capable of binding to human Axl (SEQ ID NO: 1), comprising
a heavy chain variable (VH) region comprising the CDR1, CDR2, and CDR3 sequences of SEQ ID Nos.: 19, 20, and 21, respectively, and
a light chain variable (VL) region comprising the CDR1, CDR2, and CDR3 sequences of SEQ ID Nos.: 22, GAS, and 23, respectively;

for use in treating cancer in a subject, wherein

the cancer is non-small cell lung cancer (NSCLC), and

the conjugate is administered to the subject at a dose of about 1.8-about 2.6 mg/kg body weight once every three weeks or by weekly dosing of about 0.8-about 1.2 mg/kg body weight for three weeks, optionally followed by one treatment-free week.

The present disclosure further comprises the following items:

The conjugate is administered to the subject at a dose of about 2.0-about 2.4 mg/kg body weight once every three weeks or by weekly dosing of about 0.6-about 1.4 mg/kg body weight for three weeks, optionally followed by one treatment-free week.

The conjugate may be administered to the subject at a dose of about 2.2 mg/kg body weight once every three weeks or by weekly dosing of about 1.0 mg/kg body weight for three weeks, optionally followed by one treatment-free week.

The conjugate may be administered to the subject by weekly dosing of about 0.4-1.0 mg/kg body weight.

The conjugate may be administered to the subject by weekly dosing of about 0.6-1.0 mg/kg body weight.

The conjugate may be administered to the subject by weekly dosing of about 0.4-0.8 mg/kg body weight.

The conjugate may be administered to the subject by weekly dosing of about 0.5-0.7 mg/kg body weight.

The conjugate may be administered to the subject by weekly dosing of about 0.6 mg/kg body weight.

The route of administration may in particular intravenous.

The treatment according to the invention may be continued at least until said subject has experienced progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the first dose of the conjugate.

The treatment may be continued until disease progression or unacceptable toxicity.

The non-small cell lung cancer may in particular be an adenocarcinoma.

The non-small cell lung cancer may be characterized by, and/or the subject receiving the treatment may have, one or more sensitizing mutation(s) in the epidermal growth factor receptor (EGFR) amino acid sequence (SEQ ID NO: 3).

The sensitizing mutation in the epidermal growth factor receptor (EGFR) amino acid sequence may be selected from the group consisting of:
i) An in-frame deletion and optionally insertion of one or more amino acids at position 746-751, such as any of the deletions and insertions defined in table 2,
ii) Substitution of a single amino acid at any one of positions 709, 715, 719, 720, 768, 858 and 861 such as any of the deletions and insertions defined in table 3, and
iii) An in-frame duplication and/or insertion selected from the duplications/insertions defined in Table 4;
amino acid numbering referring to the numbering of amino acids in SEQ ID NO: 3.

TABLE 2
In-frame deletions within exon 19 of the human EGFR gene
(Adapted from Shigematsu et al., Clinical and Biological
Features Associated With Epidermal Growth Factor Receptor
Gene Mutations in Lung Cancers, JNCI: Journal of the National
Cancer Institute, Volume 97, Issue 5, 2 Mar. 2005). del =
deletion; ins = insertion.

| Designation | Amino acid change |
|---|---|
| Δ1 | E746-A750 del |
| Δ2 | E746-A750 del |
| Δ3 | L747-T751 del |
| Δ4 | L747-E749 del P ins |
| Δ5 | L747-T750 del P ins |
| Δ6 | L747-S752 del S ins |
| Δ7 | E746-T751 del V ins |
| Δ8 | L747-S752 del |
| Δ9 | E746-T751 del I ins |
| Δ10 | E746-A750 del V ins |
| Δ11 | L747-S752 del Q ins |

TABLE 3
Single nucleotide substitutions and resulting amino acid
changes within exon 21 of the human EGFR gene (Adapted
from Shigematsu et al., Clinical and Biological Features
Associated With Epidermal Growth Factor Receptor Gene
Mutations in Lung Cancers, JNCI: Journal of the National
Cancer Institute, Volume 97, Issue 5, 2 Mar. 2005).

| Designation | Amino acid change |
|---|---|
| M1 | L858R |
| M2 | E709V |
| M3 | I715S |
| M4 | G719C |
| M5 | G719S |
| M6 | G719A |
| M7 | S720F |
| M8 | S768I |
| M9 | L861Q |

TABLE 4
In-frame duplications and/or insertions within exon 20 of the human
EGFR gene (Adapted from Shigematsu et al., Clinical and Biological
Features Associated With Epidermal Growth Factor Receptor Gene
Mutations in Lung Cancers, JNCI: Journal of the National Cancer
Institute, Volume 97, Issue 5, 2 Mar. 2005). ins = insertion.

| Designation | Amino acid change |
|---|---|
| D1 | ASV770-772 ins |
| D2 | H774 ins |
| D3 | G771 ins |
| D4 | CV770-771 ins |
| D5 | NP773-774 ins, H775Y |
| D6 | PH774-775 ins |
| D7 | NPH774-776 ins |
| D8 | HV775-776 ins |

The non-small cell lung cancer may be characterized by, and/or the subject receiving the treatment may have, at least one mutation in the EGFR amino acid sequence selected from L747S, D761Y, T790M, C797S, T854A, such as T790M, C797S, D761Y, and double mutations T790M/D761Y and T790/C797S; amino acid numbering referring to the numbering of amino acids in SEQ ID NO: 3.

The non-small cell lung cancer may be characterized by, and/or the subject receiving the treatment may have, at least one mutation in the EGFR amino acid sequence, which induces or confers resistance of said subject to one or more EGFR tysrosine kinase inhibitors (EGFR-TKIs).

The EGFR-TKI may be a first generation EGFR-TKI, a second generation EGFR-TKI or a third generation EGFR-TKI.

The one or more EGFR-TKIs may be selected from the group consisting of erlotinib, osimertinib, gefintinib, olmutinib, nazartinib and avitinib.

The non-small cell lung cancer may in particular be a cancer which is not characterized by a sensitizing epidermal growth factor receptor (EGFR) mutation. Likewise, the subject receiving the treatment may be a subject that does not have by a sensitizing epidermal growth factor receptor (EGFR) mutation.

The non-small cell lung cancer may be characterized by expression of an epidermal growth factor receptor (EGFR) selected form the group consisting of:
i. a wild-type human EGFR; e.g. a human EFGR that comprises the sequence set forth in SEQ ID NO: 3 or a mature polypeptide thereof; and
ii. a human EGFR which is a variant of the EGFR in item i and which, when compared with the EGFR in item I, does not have any sensitizing mutations.

The the non-small cell lung cancer may be a cancer which is not characterized by a sensitizing epidermal growth factor receptor (EGFR) mutation selected from the group consisting of:
i) An in-frame deletion and optionally insertion of one or more amino acids at position 746-751, such as any of the deletions and insertions defined in table 2,
ii) Substitution of a single amino acid at any one of positions 709, 715, 719, 720, 768, 858 and 861 such as any of the deletions and insertions defined in table 3, and
iii) An In-frame duplication and/or insertion selected from the duplications/insertions defined in Table 4;

amino acid numbering referring to the numbering of amino acids in SEQ ID NO: 3. Likewise, the subject receiving treatment according to the invention may be a subject that does not have such a sensitizing EGFR mutation.

The non-small cell lung cancer may be a cancer which is not characterized by having a mutation in the EGFR amino acid sequence, which induces or confers resistance of said subject to one or more EGFR tysrosine kinase inhibitors (EGFR-TKIs), and/or the subject does not have such a mutation. Likewise, the subject receiving treatment according to the invention may be a subject, that does not have a mutation in the EGFR amino acid sequence, which induces or confers resistance of said subject to one or more EGFR-TKIs.

The non-small cell lung cancer may be a cancer, which is not characterized by a mutation in the EGFR amino acid sequence selected from L747S, D761Y, T790M, C797S, T854A, such as from T790M, C797S, D761Y, and double mutations T790M/D761Y and T790/C797S; amino acid numbering referring to the numbering of amino acids in SEQ ID NO: 3. Likewise, the subject receiving treatment according to the invention may be a subject that does not have any of the said mutations.

The non-small cell lung cancer and/or the subject receiving treatment according to the invention may be characterized by having a mutation in the gene coding for the ALK tyrosine kinase (ALK), which leads to rearrangement of the gene coding for ALK (SEQ ID NO: 4) with a gene coding for a fusion partner, to form a fusion oncogene.

The non-small cell lung cancer may be characterized by, and/or the subject receiving treatment according to the invention may have a mutation in the gene coding the ALK, said mutation leading to rearrangement of the gene coding for ALK with the gene (EML4) coding for Echinoderm microtubule-associated protein-like 4 (EMAPL4) (SEQ ID NO: 5) (and formation of an EML4-ALK fusion oncogene).

The non-small cell lung cancer may be characterized by, and/or the subject receiving treatment according to the invention may have a mutation in the gene coding for the ALK tyrosine kinase (ALK), leading to rearrangement of the gene coding for the ALK with a gene selected from the group consisting of
i. KIF5B coding for Kinesin-1 heavy chain (KINH) (SEQ ID NO: 6),
ii. KLC1 coding for Kinesin light chain 1 (KLC1) (SEQ ID NO: 7),
iii. TFG coding for Protein TFG (SEQ ID NO: 8),
iv. TPR coding for Nucleoprotein TPR (SEQ ID NO: 9),
v. HIP1 coding for Huntington-interacting protein 1 (HIP-1) (SEQ ID NO: 10),
vi. STRN coding for Striatin (SEQ ID NO: 11),
vii. DCTN1 coding for dynactin subunit 1 (SEQ ID NO: 12),
viii. SQSTM1 coding for sequestosome-1 (SEQ ID NO: 13),
ix. NPM1 coding for nucleophosmin (SEQ ID NO: 14),
x. BCL11A coding for B-cell lymphoma/leukemia 11A (SEQ ID NO: 15), and
xi. BIRC6 coding for baculoviral IAP repeat-containing protein (SEQ ID NO: 16);

and formation of the respective fusion oncogene selected from the group consisting of a KIF5B-ALK fusion oncogene, a KLC1-ALK fusion oncogene, a TFG-ALK fusion oncogene, a TPR-ALK fusion oncogene, an HIP1-ALK fusion oncogene, a STRN-ALK fusion oncogene, a DCTN1-ALK fusion oncogene, a SQSTM1-ALK fusion oncogene, a NPM1-ALK fusion oncogene, a BCL11A-ALK fusion oncogene and a BIRC6-ALK fusion oncogene.

The non-small cell lung cancer may be characterized by expression of a wild-type human ALK tyrosine kinase; e.g. a human ALK tyrosine kinase that comprises the sequence set forth in SEQ ID NO: 4 or a mature polypeptide thereof.

The non-small cell lung cancer may be characterized by not having a mutation in the gene coding for the ALK tyrosine kinase (ALK), leading to rearrangement of ALK with fusion partner to form a fusion oncogene and/or the subject does not have such a mutation.

The non-small cell lung cancer may be characterized by not having a mutation in the gene coding for the ALK tyrosine kinase (ALK), leading to rearrangement of the gene (EML4) coding for Echinoderm microtubule-associated protein-like 4 (EMAPL4) (SEQ ID NO: 5) with ALK (SEQ ID NO: 4) and formation of an EML4-ALK fusion oncogene and/or the subject may be a subject that does not have such a mutation.

The non-small cell lung cancer may be characterized by not having a mutation in any gene selected from the group consisting of the gene coding for the ALK tyrosine kinase (ALK), the gene (EML4) coding for Echinoderm microtubule-associated protein-like 4 (EMAPL4) (SEQ ID NO: 5).

The non-small cell lung cancer may be a cancer that is not characterized by a mutation selected from the group consisting of
a sensitizing epidermal growth factor receptor (EGFR) mutation,
a mutation in the gene coding for the ALK tyrosine kinase (ALK), leading to rearrangement of EML4 with ALK and formation of an EML4-ALK fusion oncogene,
a mutation in the EGFR amino acid sequence, which induces or confers resistance of said subject to one or more EGFR tysrosine kinase inhibitors (EGFR-TKIs); and the subject may have been treated with a programmed cell death-1 (PD-1)/programmed cell death-1 (PD-1) inhibitor (e.g. nivolumab, genolimzumab, atezolizumab, durvalumab or avelumab) or with chemotherapy (e.g. chemotherapy comprising platinum, a taxane, pemetrexed and/or gemcitabine) and may have failed with such previous treatment.

The non-small cell lung cancer may be characterized by a mutation selected from the group consisting of
an sensitizing epidermal growth factor receptor (EGFR) mutation,
a mutation in the EGFR amino acid sequence, which induces or confers resistance of said subject to one or more EGFR tysrosine kinase inhibitors (EGFR-TKIs),
a mutation in the gene coding for the ALK tyrosine kinase (ALK), leading to rearrangement of EML4 with ALK and formation of an EML4-ALK fusion oncogene; and

the subject may have been treated with an EGFR inhibitor (e.g. erlotinib, osimertinib, gefintinib, olmutinib, nazartinib and avitinib) or with a PD-1/PD-L1 inhibitor (e.g. nivolumab, genolimzumab, atezolizumab, durvalumab or avelumab) and has failed with such previous treatment.

The antibody may comprise a VH region which is at least 90%, such as at least 95%, such as at least 97%, such as at least 99% identical to SEQ ID No: 17 and a VL region which is at least 90%, such as at least 95%, such as at least 97%, such as at least 99% identical to SEQ ID No: 18.

The antibody may comprise a VH region comprising SEQ ID No: 17 and a VL region comprising SEQ ID No: 18.

The antibody may be a monoclonal antibody or a monoclonal antigen-binding fragment thereof.

The antibody may be a humanized or human antibody.

The antibody may be an IgG1, such as human IgG1, optionally allotype IgG1m(f).

The antibody may be enapotamab.

The conjugate for use according to any one of the preceding items, further comprising a linker between the antibody or antigen-binding fragment and the monomethyl auristatin.

The linker may be a cleavable linker.

MMAE may be linked to the antibody with a linker, which is maleimidocaproyl-valine-citrulline-p-aminobenzyloxycarbonyl (mc-vc-PAB).

The linker may have formula -MC-vc-PAB-, wherein:
a) MC is:

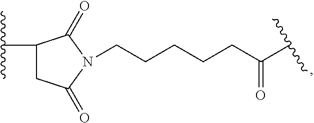 [SMILES: *C(=O)CCCCCN1C(=O)CC(*)C1=O]

b) vc is the dipeptide valine-citrulline, and
c) PAB is:

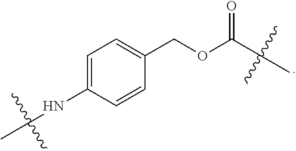 [SMILES: *Nc1ccc(COC(*)=O)cc1]

The linker may be attached to MMAE (vcMMAE), wherein vcMMAE is:

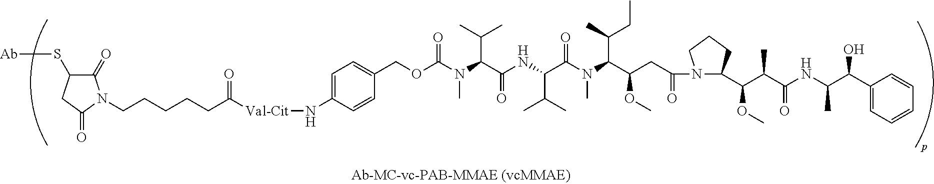

wherein p denotes a number from 1 to 8, S represents a sulphydryl residue of the antibody, and Ab designates the antibody or antigen-binding fragment thereof.

The average value of p in a population of the antibody-drug conjugate may be about 4.

The conjugate may be enapotamab vedotin.

The invention further provides a method of treating a cancer in a subject, the method comprising administering to the subject a conjugate of monomethyl auristatin or a functional analog or derivative thereof and an antibody or antigen-binding fragment thereof capable of binding to human Axl (SEQ ID NO: 1), comprising
a heavy chain variable (VH) region comprising the CDR1, CDR2, and CDR3 sequences of SEQ ID Nos.: 19, 20, and 21, respectively, and
a light chain variable (VL) region comprising the CDR1, CDR2, and CDR3 sequences of SEQ ID Nos.: 22, GAS, and 23, respectively; wherein

the cancer is non-small cell lung cancer (NSCLC), and

the conjugate is administered to the subject at a dose of about 1.8-about 2.6 mg/kg body weight once every three weeks or by weekly dosing of about 0.8-about 1.2 mg/kg body weight for three weeks, optionally followed by one treatment-free week.

The conjugate may be administered to the subject at a dose of about 2.0-about 2.4 mg/kg body weight once every three weeks or by weekly dosing of about 0.6-about 1.4 mg/kg body weight for three weeks, optionally followed by one treatment-free week.

The conjugate may be administered to the subject at a dose of about 2.2 mg/kg body weight once every three weeks or by weekly dosing of about 1.0 mg/kg body weight for three weeks, optionally followed by one treatment-free week.

The conjugate may be administered to the subject by weekly dosing of about 0.4-1.0 mg/kg body weight.

The conjugate may be administered to the subject by weekly dosing of about 0.6-1.0 mg/kg body weight.

The conjugate may be administered to the subject by weekly dosing of about 0.4-0.8 mg/kg body weight.

The conjugate may be administered to the subject by weekly dosing of about 0.5-0.7 mg/kg body weight.

The conjugate may be administered to the subject by weekly dosing of about 0.6 mg/kg body weight.

The route of administration may be intravenous.

The treatment may be continued at least until said subject has experienced progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the first dose of the conjugate.

The treatment may be continued until disease progression or unacceptable toxicity.

The non-small cell lung cancer may be an adenocarcinoma.

The non-small cell lung cancer may be characterized by, and/or the subject receiving treatment according to the invention may have one or more sensitizing mutation(s) in the epidermal growth factor receptor (EGFR) amino acid sequence (SEQ ID NO: 3).

The sensitizing mutation in the epidermal growth factor receptor (EGFR) amino acid sequence may be selected from the group consisting of:
i) An in-frame deletion and optionally insertion of one or more amino acids at position 746-751, such as any of the deletions and insertions defined in table 2,
ii) Substitution of a single amino acid at any one of positions 709, 715, 719, 720, 768, 858 and 861 such as any of the deletions and insertions defined in table 3, and
iii) An In-frame duplication and/or insertion selected from the duplications/insertions defined in Table 4;

amino acid numbering referring to the numbering of amino acids in SEQ ID NO: 3.

The non-small cell lung cancer may be characterized by, and/or the subject receiving treatment according to the invention has, at least one mutation in the EGFR amino acid sequence selected from L747S, D761Y, T790M, C797S, T854A, such as T790M, C797S, D761Y, and double mutations T790M/D761Y and T790/C797S; amino acid numbering referring to the numbering of amino acids in SEQ ID NO: 3.

The non-small cell lung cancer may be characterized by, and/or the subject receiving treatment according to the invention may have, at least one mutation in the EGFR amino acid sequence, which induces or confers resistance of said subject to one or more EGFR tysrosine kinase inhibitors (EGFR-TKIs).

The EGFR-TKI may be a first generation EGFR-TKI, a second generation EGFR-TKI or a third generation EGFR-TKI.

The one or more EGFR-TKIs may be selected from the group consisting of erlotinib, osimertinib, gefintinib, olmutinib, nazartinib and avitinib.

The non-small cell lung cancer may be a cancer that is not characterized by a sensitizing epidermal growth factor receptor (EGFR) mutation. Likewise, the subject may be a subject that does not have a sensitizing epidermal growth factor receptor (EGFR) mutation.

The non-small cell lung cancer may be a cancer that is not characterized by a sensitizing epidermal growth factor receptor (EGFR) mutation selected from the group consisting of:
i) An in-frame deletion and optionally insertion of one or more amino acids at position 746-751, such as any of the deletions and insertions defined in table 2,
ii) Substitution of a single amino acid at any one of positions 709, 715, 719, 720, 768, 858 and 861 such as any of the deletions and insertions defined in table 3, and
iii) An In-frame duplication and/or insertion selected from the duplications/insertions defined in Table 4;

amino acid numbering referring to the numbering of amino acids in SEQ ID NO: 3. Likewise, the subject receiving treatment according to the invention may be a subject that does not have a sensitizing epidermal growth factor receptor (EGFR) mutation selected from the said group.

The non-small cell lung cancer may be a cancer, which is not characterized by having a mutation in the EGFR amino acid sequence, which induces or confers resistance of said subject to one or more EGFR tysrosine kinase inhibitors (EGFR-TKIs). Likewise, the subject receiving treatment according to the invention may be subject that does not have such a mutation.

The non-small cell lung cancer may be a cancer that is not characterized by a mutation in the EGFR amino acid sequence selected from L747S, D761Y, T790M, C797S, T854A, such as from T790M, C797S, D761Y, and double mutations T790M/D761Y and T790/C797S; amino acid numbering referring to the numbering of amino acids in SEQ ID NO: 3. Likewise, the subject receiving treatment according to the invention may be a subject that does not have a mutation in the EGFR amino acid sequence selected from L747S, D761Y, T790M, C797S, T854A, such as from T790M, C797S, D761Y, and double mutations T790M/D761Y and T790/C797S; amino acid numbering referring to the numbering of amino acids in SEQ ID NO: 3.

The non-small cell lung cancer may be characterized by having a mutation in the gene coding for the ALK tyrosine kinase (ALK), which leads to rearrangement of the gene coding for ALK (SEQ ID NO: 4) with a gene coding for a fusion partner, to form a fusion oncogene.

The non-small cell lung cancer may be characterized by a mutation in the gene coding the ALK, said mutation leading to rearrangement of the gene coding for ALK with the gene (EML4) coding for Echinoderm microtubule-associated protein-like 4 (EMAPL4) (SEQ ID NO: 5) (and formation of an EML4-ALK fusion oncogene). Likewise, the subject receiving treatment according to the invention may be a subject that has a mutation in the gene coding the ALK, said mutation leading to rearrangement of the gene coding for ALK with the gene (EML4) coding for Echinoderm microtubule-associated protein-like 4 (EMAPL4) (SEQ ID NO: 5) (and formation of an EML4-ALK fusion oncogene).

The non-small cell lung cancer may be a cancer that is characterized by a mutation in the gene coding for the ALK tyrosine kinase (ALK), leading to rearrangement of the gene coding for the ALK with a gene selected from the group consisting of
i. KIF5B coding for Kinesin-1 heavy chain (KINH) (SEQ ID NO: 6),
ii. KLC1 coding for Kinesin light chain 1 (KLC1) (SEQ ID NO: 7),
iii. TFG coding for Protein TFG (SEQ ID NO: 8),
iv. TPR coding for Nucleoprotein TPR(SEQ ID NO: 9),
v. HIP1 coding for Huntington-interacting protein 1 (HIP-1) (SEQ ID NO: 10),
vi. STRN coding for Striatin (SEQ ID NO: 11),
vii. DCTN1 coding for dynactin subunit 1 (SEQ ID NO: 12),
viii. SQSTM1 coding for sequestosome-1 (SEQ ID NO: 13),
ix. NPM1 coding for nucleophosmin (SEQ ID NO: 14),
x. BCL11A coding for B-cell lymphoma/leukemia 11A (SEQ ID NO: 15), and
xi. BIRC6 coding for baculoviral IAP repeat-containing protein (SEQ ID NO: 16);

and formation of the respective fusion oncogene selected from the group consisting of a KIF5B-ALK fusion oncogene, a KLC1-ALK fusion oncogene, a TFG-ALK fusion oncogene, a TPR-ALK fusion oncogene, an HIP1-ALK fusion oncogene, a STRN-ALK fusion oncogene, a DCTN1-ALK fusion oncogene, a SQSTM1-ALK fusion oncogene, a NPM1-ALK fusion oncogene, a BCL11A-ALK fusion oncogene and a BIRC6-ALK fusion oncogene. Likewise, the subject receiving treatment according to the invention may be a subject that has a mutation as defined above.

The non-small cell lung cancer may be characterized by not having a mutation in the gene coding for the ALK tyrosine kinase (ALK), leading to rearrangement of ALK with fusion partner to form a fusion oncogene. Likewise, the subject receiving treatment according to the invention may be a subject that does not have such a mutation.

The non-small cell lung cancer may be characterized by not having a mutation in the gene coding for the ALK tyrosine kinase (ALK), leading to rearrangement of the gene (EML4) coding for Echinoderm microtubule-associated protein-like 4 (EMAPL4) (SEQ ID NO: 5) with ALK (SEQ ID NO: 4) and formation of an EML4-ALK fusion oncogene. Likewise, the subject receiving treatment according to the invention may be a subject that does not have such a mutation.

The non-small cell lung cancer may be characterized by not having a mutation in any of the genes defined above.

The non-small cell lung cancer may be a cancer that is not characterized by a mutation selected from the group consisting of
an activating epidermal growth factor receptor (EGFR) mutation,
a mutation in the gene coding for the ALK tyrosine kinase (ALK), leading to rearrangement of EML4 with ALK and formation of an EML4-ALK fusion oncogene,
a mutation in the EGFR amino acid sequence, which induces or confers resistance of said subject to one or more EGFR tysrosine kinase inhibitors (EGFR-TKIs); and

the subject may have been treated with a programmed cell death-1 (PD-1)/programmed cell death-1 (PD-1) inhibitor (e.g. nivolumab, genolimzumab, atezolizumab, durvalumab or avelumab) or with chemotherapy (e.g. chemotherapy comprising platinum, a taxane, pemetrexed and/or gemcitabine) and may have failed with such previous treatment.

The non-small cell lung cancer may be characterized by a mutation selected from the group consisting of
an activating epidermal growth factor receptor (EGFR) mutation,
a mutation in the EGFR amino acid sequence, which induces or confers resistance of said subject to one or more EGFR tysrosine kinase inhibitors (EGFR-TKIs),
a mutation in the gene coding for the ALK tyrosine kinase (ALK), leading to rearrangement of EML4 with ALK and formation of an EML4-ALK fusion oncogene; and

the subject may have been treated with an EGFR inhibitor (e.g. erlotinib, osimertinib, gefintinib, olmutinib, nazartinib and avitinib) or with a PD-1/PD-L1 inhibitor (e.g. nivolumab, genolimzumab, atezolizumab, durvalumab or avelumab) and has failed with such previous treatment.

The antibody may comprise a VH region, which is at least 90%, such as at least 95%, such as at least 97%, such as at least 99% identical to SEQ ID No: 17 and a VL region which is at least 90%, such as at least 95%, such as at least 97%, such as at least 99% identical to SEQ ID No: 18.

The antibody may comprise a VH region comprising SEQ ID No: 17 and a VL region comprising SEQ ID No: 18.

The antibody may be a monoclonal antibody or a monoclonal antigen-binding fragment thereof.

The antibody may be a humanized or human antibody.

The antibody may be an IgG1, such as human IgG1, optionally allotype IgG1m(f).

The antibody may be enapotamab.

The conjugate used in the method according to the invention may comprise a linker between the antibody or antigen-binding fragment and the monomethyl auristatin.

The linker may be a cleavable linker.

MMAE may be linked to the antibody with a linker, which is maleimidocaproyl-valine-citrulline-p-aminobenzyloxycarbonyl (mc-vc-PAB).

The linker may have the formula -MC-vc-PAB-, wherein:
a) MC is:

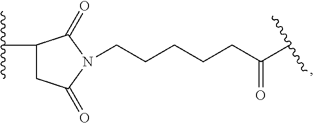 [SMILES: *C(=O)CCCCCN1C(=O)CC(*)C1=O]

b) vc is the dipeptide valine-citrulline, and
c) PAB is:

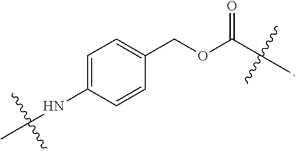 [SMILES: *Nc1ccc(COC(*)=O)cc1]

The linker may be attached to MMAE (vcMMAE), wherein vcMMAE is:

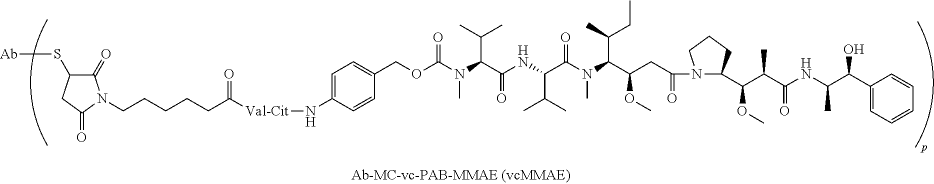

wherein p denotes a number from 1 to 8, S represents a sulphydryl residue of the antibody, and Ab designates the antibody or antigen-binding fragment thereof.

The average value of p in a population of the antibody-drug conjugate my in particular be 4, such as about 4.

The conjugate may be enapotamab vedotin.

The invention further provides a kit comprising a conjugate of monomethyl auristatin or a functional analog or derivative thereof and an antibody or antigen-binding fragment thereof capable of binding to human Axl (SEQ ID NO: 1), comprising
a heavy chain variable (VH) region comprising the CDR1, CDR2, and CDR3 sequences of SEQ ID Nos.: 19, 20, and 21, respectively, and
a light chain variable (VL) region comprising the CDR1, CDR2, and CDR3 sequences of SEQ ID Nos.: 22, GAS, and 23, respectively;

and instructions for using the conjugate as set forth in any one of the preceding items.

The present invention is further illustrated by the following examples which should not be construed as further limiting the scope of the present disclosure.

### EXAMPLES

### Example 1: First-in-Human, Open-Label, Dose-Escalation Trial with Expansion Cohorts to Evaluate Safety of Axl-Specific Antibody-Drug Conjugate (HuMax®-AXL-ADC) in Patients with Solid Tumors

The present study was an open label, multi-center Phase I/IIa safety trial of HuMax AXL ADC in a mixed population of patients with solid tumors known from the literature to overexpress Axl and where the use of systemic tubulin inhibitors is part of Standard of Care (SoC). The trial consisted of two parts; a dose escalation part (phase I, first-in-human (FIH)) and an expansion part (phase IIa).

The dose escalation part consists of two, staggered, arms for identification of the most optimal dosing regimen:
1Q3W: Dosing once every 3 weeks
3Q4W: Weekly dosing for 3 weeks followed by one treatment-free week.

The aim of the expansion part of the study was to provide further data on the safety, tolerability, PK and anti-tumor activity of the selected dose.

HuMax®-AXL-ADC (Enapotamab vedotin) was produced as described previously, e.g. in WO 2016/005593 (PCT/EP2015/065900).

The antibody moiety of HuMax®-AXL-ADC comprises a heavy chain variable (VH) region comprising the CDR1, CDR2, and CDR3 sequences of SEQ ID Nos.: 19, 20, and 21, respectively, and a light chain variable (VL) region comprising the CDR1, CDR2, and CDR3 sequences of SEQ ID Nos.: 22, GAS, and 23, respectively. In brief, monoclonal anti-Axl antibodies were generated by immunization of transgenic mice producing fully human antibodies (Medarax). The antibodies were cloned and expressed a IgG1-kappa. Monoclonal antibodies were conjugated to Monomethyl auristatin E (MMAE) via a cleavable maleimidocaproyl-valyl-citrullinyl-p-aminobenzyloxycarbonyl (mc-val-cit-PABC) type linker.

Inclusion Criteria:

Patients had to meet all of the following inclusion criteria before they will be allowed to participate in the trial:
1. For the dose escalation part: Patients with relapsed or refractory cancer of the ovary, cervix, endometrium, thyroid, non-small cell lung cancer (NSCLC), or melanoma (cutaneous, mucosal, acral or uveal melanoma) who have failed available standard therapy or who are not candidates for standard therapy, and for whom, in the opinion of the investigator, experimental therapy with HuMax-AXL-ADC may be beneficial.
   For the expansion part: Patients with relapsed or refractory, advanced and/or metastatic cancer who were not candidates for standard therapy, and for whom, in the opinion of the investigator, experimental therapy with HuMax-AXL-ADC could be beneficial. The expansion part had a total of seven cohorts or “arms”. Cohorts 1 and 2 included non-small cell lung cancer patients who had failed anticancer therapy as follows:
   Expansion Cohort 1 (NSCLC patients with classical sensitizing EGFR mutations and/or other EGFR resistance mutations targeted by third generation TKIs (e.g. T790M for osimertinib)):
      NSCLC patients after failure of up to 4 prior treatment regimens containing systemic therapy for metastatic disease
         adjuvant and maintenance treatment is considered being part of one treatment regimen.
      Documented progressive disease on or after last prior treatment
      Last prior treatment to enrolment to GCT1021-01 should have been:
         an EGFR inhibitor (e.g. Erlotinib, Osimertinib, etc.),
         or a PD-1/PD-L1 inhibitor,
         or a platinum-based doublet chemotherapy.
   Expansion Cohort 2 (NSCLC patients without sensitizing EGFR mutations or ALK rearrangements)
      NSCLC patients after failure of up to 4 prior treatment regimens containing systemic therapy for metastatic disease.
         adjuvant and maintenance treatment is considered being part of one treatment regimen.
      Documented progressive disease on or after last prior treatment.
      Last prior treatment to enrolment to GCT1021-01 should have been
         a PD-1/PD-L1 inhibitor.
         or a chemotherapy (containing platinum, and/or taxane, and/or pemetrexed, and/or gemcitabine).
   For the following conditions in Expansion Cohorts 1-2, the sponsor medical officer's approval of enrolment was needed:
      if documented progression had not been on measurable disease (i.e. symptomatic progression).
      if last prior treatment to enrolment was a combination therapy which contained an EGFR inhibitor or a PD-1/PD-L1 inhibitor.
      if last prior treatment to enrolment contain chemotherapies other than platinum, and/or taxane, and/or pemetrexed, and/or gemcitabine (only applicable for NSCLC patients without sensitizing EGFR mutations or ALK rearrangements).
2. Patients were required to have measurable disease according to RECIST (Response Evaluation Criteria In Solid Tumors) version 1.1.
   A minimum of one lesion ≥10 mm (or twice the slice thickness if slices are not 5 mm thick) in the longest diameter (LD) from a non-irradiated area
      Lymph nodes lesion 15 mm in the shortest diameter from a non-irradiated area.
      If target lesion(s) are located within previously irradiated area patients can be enrolled if:
         target lesions have not been irradiated within the last 3 months.
         there has been demonstrated progression in the “in field” target lesion and after sponsor acceptance.
   In the dose escalation part, patients with ovarian cancer could be included based on CA 125 positivity according to the Gynecologic Cancer Intergroup Guideline^{1,2}; only if they had a pretreatment sample that was at least twice the upper limit of the reference range and within 2 weeks before starting the treatment.
   Note: Patients were not evaluable by CA 125 if they had received mouse antibodies (unless the assay used had been shown not to be influenced by human anti-mouse antibody) or if there had been medical and/or surgical interference with their peritoneum or pleura during the previous 28 days (e.g. paracentesis).
3. In the dose escalation part all patients were required provide a tumor tissue sample (Formalin Fixed Paraffin Embedded blocks/slides) from archival tissue or fresh biopsy collected before Cycle 1, Visit 1, preferably derived from advanced disease stage. In the expansion part all patients were required to provide a fresh biopsy (aspirates were not acceptable) taken after failure/stop of last prior treatment. Furthermore the latest available tumor tissue sample had to be collected if available.
4. Age 18 years.
5. Have an acceptable renal function defined as:
   Glomerular filtration rate (GFR)≥40 mL/min/1.73 m²—e.g., according to the abbreviated Modification of Diet in Renal Disease (MDRD) equation: GFR=186×(SCr^{-1.154})×(age^{-0.203})(where SCr, the serum creatinine level, is expressed in mg/dL; multiply it by 0.742 if the patient was female; multiply it by 1.212, if the patient was African-American³).
   Not being on dialysis.
6. Have an acceptable liver function defined as:
   Alanine aminotransferase (ALT) and aspartate aminotransferase (AST)≤3 times the ULN; if liver tumor/metastases were present, then ≤5×ULN was allowed.
   Bilirubin≤1.5×ULN, except in patients diagnosed with Gilbert's syndrome, direct bilirubin≤2×ULN.
7. Have an acceptable hematological status defined as:
   Hemoglobin 5.6 mmol/L (˜9 g/dL).
   Absolute neutrophil count (ANC)≥1500/μL (1.5×10⁹/L).
   Platelet count≥100×10⁹/L.
8. Have an Eastern Cooperative Oncology Group (ECOG) performance status of 0 or 1.
9. Life expectancy of at least three months.
10. Patients, both females and males, of childbearing/reproductive potential had to agree to use adequate contraception while included in the trial and for six months after the last infusion of HuMax-AXL-ADC.
11. Patients were required to provide signed informed consent form.

Exclusion Criteria:

If any of the following applied, the patient could not enter the trial:

Hematological
1. Acute deep vein thrombosis or clinically relevant pulmonary embolism, not stable for at least 4 weeks prior to first Investigational Medicinal Product (IMP) administration.

Cardiovascular
2. Have clinically significant cardiac disease, including:
   Onset of unstable angina within six months of signing the Informed Consent Form (ICF).
   Acute myocardial infarction within six months of the signing the ICF.
3. Known congestive heart failure (Grade III or IV as classified by the New York Heart Association); and/or a known decreased cardiac ejection fraction of <45%.A baseline QT interval as corrected by Fridericia's formula (QTcF)>480 msec, or a complete left bundle branch block (defined as a QRS interval≥120 msec in left bundle branch block form).
4. Uncontrolled hypertension defined as systolic blood pressure ≥160 mmHg and/or diastolic blood pressure ≥100 mmHg, despite optimal medical management.

Excluded Medications or Treatment Regimens
5. Have received granulocyte colony stimulating factor (G-CSF) or granulocyte/macrophage colony stimulating factor support 3 weeks prior to first IMP administration.
6. Have received a cumulative dose of corticosteroid ≥150 mg prednisone (or equivalent doses of corticosteroids) within two weeks before the first IMP administration.
7. History of ≥grade 3 allergic reactions to monoclonal antibody therapy as well as known or suspected allergy or intolerance to any agent given in the course of this trial.

Surgery/Procedures
8. Major surgery within four weeks before first IMP administration.

Central Nervous System
9. Any history of intracerebral arteriovenous malformation, cerebral aneurysm, brain metastases or stroke.
   Transient ischemic attack >1 month prior to screening was allowed.
   Patients with central nervous system symptoms were required to undergo a Computed Tomography (CT) scan or Magnetic Resonance Imaging of the brain to exclude new or progressive brain metastases. Spinal cord metastasis was acceptable. However, patients with spinal cord compression were excluded.
   In the expansion cohorts the enrolment of patients with stable brain metastases, i.e. being asymptomatic for the last 14 days prior to treatment initiation, was allowed.
   Symptomatic uncontrolled brain or leptomeningeal metastases. (To be considered “controlled,” central nervous system [CNS] disease were required to have undergone treatment [eg, radiation or chemotherapy] at least 2 weeks prior to first IMP administration. The patient were required not have any new or progressive signs or symptoms related to the CNS disease and were required to take ≤10 mg of prednisone or equivalent per day or no steroids). Patients who had untreated brain metastases and who were not symptomatic could if the investigator felt that treatment of these metastases was not indicated. Patients with spinal cord compression could be considered for enrolment if they had received definitive treatment for this and evidence of clinically stable disease (SD) for 28 days.

Prior Therapy
10. Any anticancer therapy including; small molecules, immunotherapy, chemotherapy monoclonal antibodies or any other experimental drug within five half-lives but maximum four weeks before first infusion. Accepted exceptions were bisphosphonates, denosumab and gonadotropin-releasing hormone agonist or antagonist, which could be continued throughout the trial.
   Toxic effects of prior anti-cancer therapy considered as chronic, such as chemotherapy-induced fatigue, alopecia, or anorexia of ≤grade 2, where no more resolution was expected, did not prevent the patient from participation in the trial.
11. Any prior therapy with a conjugated or unconjugated auristatin derivative/vinca-binding site targeting payload. (Previous treatment with vinca alkaloids was allowed in line with inclusion criterion #1.)
12. Radiotherapy within 14 days prior to first IMP administration (Palliative radiotherapy was allowed).

Other Cancer/Metastases
13. Known past or current malignancy other than inclusion diagnosis, except for:
   Cervical carcinoma of Stage 1B or less.
   Non-invasive basal cell or squamous cell skin carcinoma.
   Non-invasive, superficial bladder cancer.
   Prostate cancer with a current PSA level <0.1 ng/mL.
   Breast cancer in BRCA1 or BRCA2 positive ovarian cancer patients.
   Any curable cancer with a complete response (CR) of >2 years duration.

Other
14. Melanoma patients with an LDH≥3×ULN.
15. Ongoing significant, uncontrolled medical condition including:
   Serious, non-healing wound, skin ulcer (of any grade), or bone fracture.
16. Presence of grade≥2 peripheral neuropathy.
17. Clinically significant active viral, bacterial or fungal infection requiring:
   Intravenous treatment with anti-infective therapy that had been administered less than two weeks prior to first dose, or
   Oral treatment with anti-infective therapy that had been administered less than one week prior to first dose.
   Prophylactic anti-infective therapy, which was given without clinical symptomatic was allowed (e.g. antibiotic prophylaxis prior to dental extraction, etc.).
18. Known human immunodeficiency virus seropositivity.
19. Known history/positive serology for hepatitis B (unless immune due to vaccination or resolved natural infection or unless passive immunization due to immunoglobulin therapy):
   Positive test for antibodies to hepatitis B core antigens (anti-HBc), and
   Negative test for antibodies to hepatitis B surface antigens (anti-HBs).
20. Known positive serology for hepatitis C (unless due to immunoglobulin therapy).
21. Substance abuse, medical, psychological or social conditions that could interfere with the patient's participation in the trial or evaluation of the trial result.
22. History of organ allograft (except for corneal transplant) or autologous or allogeneic bone marrow transplant, or stem cell rescue within 3 months prior to the first dose of IMP.
23. Body weight <40 kg.
24. Women who were pregnant or breast feeding.

Specifically for NSCLC:
25. Pulmonary hemorrhage or hemoptysis >2.5 ml blood within 6 weeks unless cause had been addressed and was medically resolved.
26. History of acute pneumonitis.

Dose Escalation and Mode of Administration:

1Q3W

The 1Q3W dose escalation evaluated HuMax-AXL-ADC at seven main dose levels: 0.3, 0.6, 1.0, 1.5, 2.0, 2.4 and 2.8 mg/kg, and 4 optional intermediate dose levels 1.25, 1.8, 2.2 and 2.6 mg/kg. Further escalation with steps of 0.4 mg/kg and de-escalation by 0.2 mg/kg was allowed, if the MTD had not been declared at a dose level up to 2.8 mg/kg.

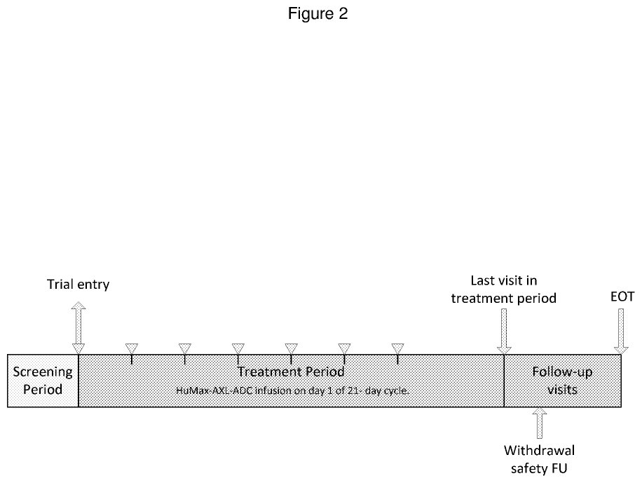

In the 1Q3W dose escalation the patients received 1 infusion of HuMax-AXL-ADC every three weeks as according to FIG. 2.

3Q4W

When a minimum of 8 patients had been treated and evaluated for Dose limiting toxicities (DLTs), the 1.5 mg/kg cohort was declared safe on the 1Q3W arm, and the predicted AUC on the starting dose in 3Q4W arm was below pre-defined limits, the 3Q4W arm was initiated.

The 3Q4W dose escalation was conducted as a standard 3 (+3) design which evaluatedHuMax-AXL-ADC at doses of (0.45), 0.6, 0.8, 1.0, 1.2 and 1.4 mg/kg. The escalation was allowed to continue to higher dose levels with increments up to 20%, if the 1.4 mg/kg was reached without significant safety concerns and it was considered safe to escalate above 1.4 mg/kg, the. The starting dose was expected to be 0.6 mg/kg (a dose level of 0.45 mg/kg could be added) and as an additional precaution, the independent Data Monitoring Committee (DMC) could recommend intermediate dose levels at any step during dose escalation.

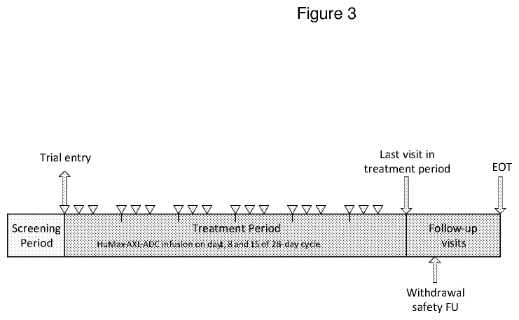

In the 3Q4W dose escalation the patients received weekly dosing for 3 weeks followed by one treatment-free week according to FIG. 3. Patients were treated until disease progression or unacceptable toxicity was observed.

Rationale for Dose Frequency

In the dose escalation part, HuMax-AXL-ADC was administered 1Q3W in the first dose escalation arm and 3Q4W in the second dose escalation arm. The dosing frequency was based on toxicokinetic and toxicology data obtained in cynomolgus monkeys, suggesting adequate recovery of neutrophils, thrombocytes and red blood cell parameters and otherwise an acceptable safety profile. No relevant accumulation of HuMax-AXL-ADC or MMAE between cycles is anticipated.

Treatment Preparation

The dose of HuMax-AXL-ADC for administration was prepared by the site pharmacy using aseptic technique. HuMax-AXL-ADC was supplied to the site/pharmacy as bulk supply cartons. Labelling of the IMP was done in accordance with local standards and regulations.

The Investigational Medicinal Product (IMP) was supplied in vials containing 40 mg of HuMax-AXL-ADC as lyophilized powder. The powder was reconstituted with 4 mL water for injection leading to a 10 mg/mL solution.

The reconstituted HuMax-AXL-ADC was diluted into 0.9% NaCl 100 mL infusion bag according to the dose assigned to the patient.

HuMax-AXL-ADC (lyophilized vials) were stored in a refrigerator at 2° C. to 8° C.

The infusion was required to be completed within 24 hours after the HuMax-AXL-ADC vials have been reconstituted. An in-line filter 0.2 μm must be used for the infusion. The entire 100 mL infusion volume from the prepared infusion bag needs to be administered, no dead volume is provided.

Treatment Administration

HuMax-AXL-ADC was administered as an intravenous infusion. Each patient's dose was calculated based on the patient's weight rounded to the nearest kilogram, i.e., assigned cohort dose in mg/kg×body weight in kg. For patients whose body mass index (BMI) was greater than 30 kg/m², the investigator was required to use a weight that, based on the patient's height, corresponds to a maximum BMI of 30.

The dose was calculated according to the following formula if BMI is greater than 30 kg/m²:

Dose (mg)=x(mg/kg)*30(kg/m2)*height (m)*height(m) (where x denotes the dose level)

HuMax-AXL-ADC was administered over a minimum of 30 minutes and the infusion must be completed within 4 hours. The infusion was complete when the infusion line has been flushed with saline.

In the dose-escalation part, there was a minimum of 2 nights between the first and second patient in each dose cohort to account for any safety concerns in each new dose.

Duration of Treatment:

Dependent on which dose-escalation arm the patient was recruited to, HuMax AXL ADC was administered either 1Q3W or 3Q4W. The patients received treatment with HuMax-AXL-ADC until disease progression or unacceptable toxicity. Patients were followed for 52 weeks after end of treatment. In the expansion part of the trial patients received HuMax AXL ADC at the maximum tolerated dose (MTD) found in either 1Q3W or 3Q4W schedule as recommended by the DMC and confirmed by the internal sponsor safety committee.

Criteria for Evaluation:

Primary Endpoints
Dose Limiting Toxicities (DLTs)
Adverse events (AEs): incidences of AEs, serious adverse events (SAEs), infusion-related AEs, grade 3 AEs, and AEs related to IMP during the trial.

Secondary Endpoints
Safety laboratory parameters (hematology and biochemistry).
PK parameters (clearance, volume of distribution and area-under-the-concentration-time curve [AUC_{0-Clast} and AUC_{0-∞}], maximum concentration [Cₘₐₓ], time of Cₘₐₓ [Tₘₐₓ], pre dose values, and half-life of HuMax-AXL-ADC and free toxin monomethyl auristatin E [MMAE]).
Immunogenicity of HuMax-AXL-ADC (anti-drug antibodies).
Anti-tumor activity measured by tumor shrinkage (based on computerized tomography [CT]-scan evaluations), as well as change in CA 125 in patients with ovarian cancer and change in prostate specific antigen (PSA) in patients with castration-resistant prostate cancer (CRPC).
Objective Response, Progression-Free Survival (PFS), Duration of Response (DoR) and Overall survival (OS).
Axl expression in the tumor biopsy.

Response

Response in solid tumor cancers was assessed in accordance with the RECIST criteria version 1.1¹⁸ and for patients with ovarian cancer according to RECIST 1.1 in combination with CA 125 as defined by the Gynecological Cancer Intergroup².

TABLE 5
Definition of Response (RECIST Criteria v1.1)

| | Category | Criteria |
|---|---|---|
| Based on target | Complete Response | Disappearance of all target lesions. Any pathological lymph |
| lesions | (CR) | nodes must have reduction in short axis to <10 mm. |
| | Partial Response | ≥30% decrease in the sum of the LD of target lesions, |
| | (PR) | taking as reference the baseline sum LD. |
| | Stable Disease | Neither sufficient shrinkage to qualify for PR nor sufficient |
| | (SD) | increase to qualify for PD, taking as reference the smallest |
| | | sum of LDs since the treatment started. |
| | Progressive Disease | ≥20% increase in the sum of the LDs of target lesions, |
| | (PD) | taking as reference the smallest sum of the LDs recorded |
| | | since the treatment started or the appearance of one or |
| | | more new lesions. |
| Based on non- | CR | Disappearance of all non-target lesions and normalization |
| target lesions | | of tumor marker level. All lymph nodes must be non- |
| | | pathological in size (<10 mm short axis). |
| | SD | Persistence of one or more non-target lesion(s) or/and |
| | | maintenance of tumor marker level above the normal |
| | | limits. |
| | PD | Appearance of one or more new lesions and/or unequivocal |
| | | progression of existing non-target lesions. |

Response Evaluation and Reporting of Results

In the dose escalation, response evaluation was performed by the investigator and sponsor. In the expansion, response evaluation was performed by the investigator and sponsor as well as a group of external medical experts. Each patient was assigned one of the following categories:

1) CR,

2) PR,

3) SD,

4) PD, or

5) Not Evaluable

Patients in response categories 1 and 2 were considered responders and patients in response categories 4 and 5 were considered as failing to respond to treatment (disease progression). Patients in response categories 1, 2 and 3 were considered to be in disease control.

Individual patient data listings and summaries of objective response, best overall tumor response (based primarily on confirmed but also on unconfirmed response) and disease control was to be presented.

For patients with ovarian cancer, responses were to be evaluated and reported as per RECIST 1.1¹⁸, CA 125 and the combination of the two sets of response criteria in accordance with the Gynecological Cancer Intergroup definitions².

For patients with prostate cancer, responses were to be evaluated and reported as per RECIST 1.1¹⁸ and PSA according to the Updated Recommendations from the Prostate Cancer Clinical Trials Working Group 3.¹⁹

Progression-Free Survival

PFS is defined as the number of days from Visit 1 in Cycle 1 to first PD or death. Only deaths that occurred within 30 days of the last progression assessment were to be considered in the analysis. If no death was observed within this period, PFS was to be censored at the last progression assessment. PFS was derived for all patients and presented graphically as well as summarized using survival analysis methods: distribution functions were to be estimated using Kaplan-Meier technique and times were to be censored in accordance with Table A in Appendix 3 in the FDA Guidance for Industry: Clinical Trial Endpoints for the Approval of Cancer Drugs and Biologics (2007).

Duration of Response

DoR is defined as the number of days from the first documentation of objective tumor response (CR or PR) to the date of first PD or death. DoR was to be analyzed using the same statistical methodology as PFS.

Overall Survival

Overall survival (OS) is defined as the number of days from Visit 1 in Cycle 1 to death. OS was analyzed using the same statistical methodology as PFS and DoR except that censoring was not applied neither when visits were skipped nor when new anti-cancer therapies were given.

Tumor Shrinkage

Tumor shrinkage (based on CT-scan evaluations) was listed and summarized, per source (radiologist, central read).

Results:

Dose Escalation

Results: 47 patients with NSCLC (n=8), melanoma (n=9), ovarian (n=22), cervical (n=3) and endometrial (n=5) cancer enrolled in ph1 (1Q3W n=32; 3Q4W n=15). Most patients were female (87%), White (94%) and aged <⁶⁵y (66%). Maximum Tolerated Dose (MTD) was 2.2 mg/kg in the 1Q3W arm and 1.0 mg/kg in the 3Q4W arm; Recommended Phase 2 Dose (RP2D) was 2.2 mg/kg for the 1Q3W dosing regime. Enapotamab Vedotin median elimination half-life was 0.9-2.2 days across doses/schedules. In the 47 patients enrolled, there were 6 DLTs (Table). The most common Adverse Effects (any grade; in ≥40% of patients) were fatigue (64%), nausea (57%), constipation (57%), diarrhea (47%), vomiting (45%) and decreased appetite (43%). Three patients in the 1Q3W arm had a partial response (1 NSCLC [2.2 mg/kg dose]; 2 ovarian [1.5 and 2.4 mg/kg dose levels]).

Conclusions: The RP2D of single agent Enapotamab Vedotin in pre-treated patients with solid tumors was 2.2 mg/kg 1Q3W. Enapotamab Vedotin showed encouraging preliminary antitumor activity.

| DLT | Dose, mg/kg (n) |
|---|---|
| 1Q3W | |
| Constipation | 2 (1); 2.2 (1) |
| Vomiting | 2.2 (1) |
| GGT increase | 2.4 (1) |
| 3Q4W | |
| Febrile neutropenia | 1.2 (1) |
| Diarrhea | 1.2 (1) |

Expansion cohort 1 (EGFR mutated NSCLC patients):

Approximately 15 month after opening of cohort 1, 18 patients had been treated with HuMax©-AXL-ADC (Enapotamab vedotin). Of the 18 patients treated
1 patient had a Best Overall Response (BoR) of unconfirmed PR
6 patients had a BoR of SD
6 patients had a BoR of PD
4 patients discontinued prior to first post baseline scan* * 3 patients withdrew consent, 1 patient discontinued due to AE (unrelated pneumonia)
1 patients have not yet reached first post baseline scan

Objective Response Rate (unconfirmed)=5.5%

Disease Control Rate (DCR)=39%

Expansion Cohort 2 (NSCLC Patients without Sensitizing EGFR Mutations or ALK Rearrangements):

Approximately 15 month after opening of cohort 1, 34 patients had been treated with HuMax©-AXL-ADC (Enapotamab vedotin) in expansion. Of the 34 patients treated in expansion:
4 patients had a BoR of confirmed PR
12 patients had a BoR of SD
7 patients had a BoR of PD
8 patients discontinued prior to first post-baseline scan*
3 patients have no post baseline scan at data cut

Objective Response Rate (ORR) (confirmed)=11%

DCR=47%

A total of 36 patients had been treated with 2.2 mg/kg (escalation+expansion). Of the 36 patients:
5 patients had a BoR of confirmed PR
13 patients had a BoR of SD
7 patients had a BoR of PD
8 patients discontinued prior to first post-baseline scan* * 2 patients discontinued due to adverse events (disease progression (unrelated) and fatigue (related), 3 due to death (pneumonia, pulmonary embolism and disease progression (all unrelated)), 1 patient withdrew consent, 1 patient discontinued due to investigators decision and 1 patient discontinued lacking reason.
3 patients have no post baseline scan at data cut

ORR (confirmed)=13.8%

DCR=50%

### Subject Examples

Subject 401

This 71 year old, white female patient was enrolled in the study GEN1021 and signed the informed consent form on the 11^{th} of April 2018 at a site in the UK.

The patient was diagnosed with stage IIIA, non-small cell lung andenocarcinoma (negative for ALK rearrangement) on the 5^{th} of August 2016.

Past cancer treatments included cisplatin plus vinorelbin from August to September 2016, reported with progression during treatment and a best response of progressive disease (PD). The patient received cisplatin plus premetrexed from October 2016 to November 2016 with a best response of partial response (PR) but treatment was discontinued due to toxicity. Patient received Erlotinb from June to August 2017 with best response of PD and last prior treatment before enrolment on GEN 1021 was pembrolizumab from September 2018 to January 2018 with a best response of stable disease (SD). Treatment with pembrolizumab was discontinued due to progression of disease.

Medical history included childhood polio and subdural hematoma, both conditions resolved at the time of enrollment. In addition the patient had peripheral neuropathy, cough and right eye cataract, all conditions ongoing at the time of enrollment. Patient is a non-smoker and was reported with an ECOG of 1 at the time of enrollment.

Patient received the first dose of enapotamab vendotin on C1D1 (20 Apr. 2018).

Treatment emerging events include urinary tract infection (G2, unrelated), creatinine kinase increase (fluctuating between G1 and G2, possibly related), muscle cramps (G1, possibly related), worsening of cough (G2, unrelated) and ALT increase and AST increase (both G1 and unrelated). For none of these events study drug administration was altered. In addition patient experienced the event of dysphonia and left leg weakness, both events reported as G1 and possibly related and due to these events, study drug administration was interrupted.

At screening two target lesions (TLs) were identified in the lungs, one in left lower lobe reported with the longest diameter of 11 mm and one in the right upper lobe reported with the longest diameter of 15 mm. The sum of diameters at screening was 26 mm. In addition, one non-target lesion (NTL) was identified in the lung (site not specified).

At C2D15 (25 May 2018), first post-baseline scan was performed. At that time, TL in the left lower lobe was reported with a diameter of 10 mm and the TL in the upper right lung with a diameter of 12 mm and thus the sum of diameters of 22 mm. As compared to screening, this corresponds to 15% decrease in sum of diameters. The NTL was reported as present (SD) and no new lesions were detected. The overall response assessment was reported as SD according to RECIST 1.1.

At C4D15 (6 Jul. 2018), the second post-baseline scan was performed. At that time, TL in the left lower lobe was reported with a diameter of 8 mm and the TL in the upper right lung with a diameter of 9 mm and thus the sum of diameters of 17 mm. As compared to screening, this corresponds to 34.6% decrease in sum of diameters. The NTL was reported as present (SD) and no new lesions were detected. The overall response assessment was reported as PR according to RECIST 1.1.

At C6D15 (17 Aug. 2018), the third post-baseline scan was performed. At that time, TL in the left lower lobe was reported with a diameter of 5 mm and the TL in the upper right lung with a diameter of 6 mm and thus the sum of diameters of 11 mm. As compared to screening, this corresponds to 57.6% decrease in sum of diameters. The NTL was reported as present (SD) and no new lesions were detected. The overall response assessment was reported as PR according to RECIST 1.1.

To date, patient is ongoing in Cycle 8 as of 21^{st} of September 2018 with a confirmed response of PR as per investigators assessment. Independent review of the tumor assessments has not yet been performed.

Subject 403

This 63 year old, white female patient was enrolled in the study GEN1021 and signed the informed consent form on the 4^{th} of May 2018 at a site in the UK.

The patient was diagnosed with stage IV, non-small cell lung andenocarcinoma (negative for EGFR mutations and ALK rearrangement) on the 19^{th} of January 2017.

Past cancer treatments included carboplatin plus pemetrexed from February 2017 to March 2017, reported with progression during treatment and a best response of PD. The patient was treated with radiotherapy in April 2017, with a best response of PR. Last prior treatment before enrolment on GEN 1021 was pembrolizumab from June 2017 to September 2017 with a best response of PD.

Medical history included cervical intraepithelial neoplasia Dizziness, light headaches and constipation, all of which were resolved at the time of enrollment. Hypertension, neck osteoarthritis, gallstones, postural hypotension, fatigue, cough, intermittent left sided chest pain, anxiety, arthralgia, anorexia and dry skin are reported as ongoing medical conditions at the time of enrollment.

Patient is a past-smoker (47 years) but discontinued smoking in January 2017. Patient was reported with an ECOG of 1 at the time of enrollment.

Patient received the first dose of enapotamab vendotin on C1D1 (15 May 2018).

Treatment emerging events include two episodes of nausea (both Gland possibly related), skin and subcutaneous tissue disorder (G1, not related), constipation (G2, related), two episodes of anorexia (G1, first episode unrelated, second episode possibly related) gastroesophageal reflux (G1, not related), alopecia (G1, related), AST increase (G1, possibly related). For none of the reported events, study treatment administration was changed.

At screening four TLs were identified. The lesions were following: Left axillary nodal mass reported with diameter of 24 mm, right lower lung lobe lesion with diameter of 15 mm, right lower lung lobe lesion with diameter of 13 mm and right iliac lesion with diameter of 36 mm. The sum of diameters at screening was 88 mm. In addition, two NTLs were identified, one in the right middle lobe of the lung and in left supraclavicular fossa lymph node.

At C2D15 (19 Jun. 2018), first post-baseline scan was performed. At that time, the left axillary nodal mass reported with diameter of 14 mm, right lower lung lobe lesion with diameter of 12 mm, right lower lung lobe lesion with diameter of 9 mm and right iliac lesion with diameter of 36 mm. The sum of diameters at C2D15 was 71 mm. As compared to screening, this corresponds to 19.3% decrease in sum of diameters. The NTLs were reported as present (SD) and no new lesions were detected. The overall response assessment was reported as SD according to RECIST 1.1.

At C4D15 (31 Jul. 2018), the second post-baseline scan was performed. At that time the left axillary nodal mass reported with diameter of 10 mm, right lower lung lobe lesion with diameter of 9 mm, right lower lung lobe lesion with diameter of 6 mm and right iliac lesion with diameter of 32 mm. The sum of diameters at C2D15 was 57 mm. As compared to screening, this corresponds to 35.2% decrease in sum of diameters. One of the two NTLs was reported as present whereas the other was reported as absent (SD) and no new lesions were detected. The overall response assessment has not been reported in the eCRF to date.

At C6D15 (11 Sep. 2018), the third post-baseline scan was performed. At that time the left axillary nodal mass reported with diameter of 10 mm, right lower lung lobe lesion with diameter of 9 mm, right lower lung lobe lesion with diameter of 7 mm and right iliac lesion with diameter of 30 mm. The sum of diameters at C2D15 was 56 mm. As compared to screening, this corresponds to 36.4% decrease in sum of diameters. To date, the status of the two NTLs has not been reported in the eCRF and no new lesions were detected. Overall TL assessment has been reported as PR, overall status of NTL has been reported as “not evaluable” but overall response assessment for this time point has not yet been reported.

To date, patient is ongoing in Cycle 7 as of 18^{th} of September 2018 with a response of PR as per investigators assessment. Independent review of the tumor assessments is ongoing but has not yet been completed.

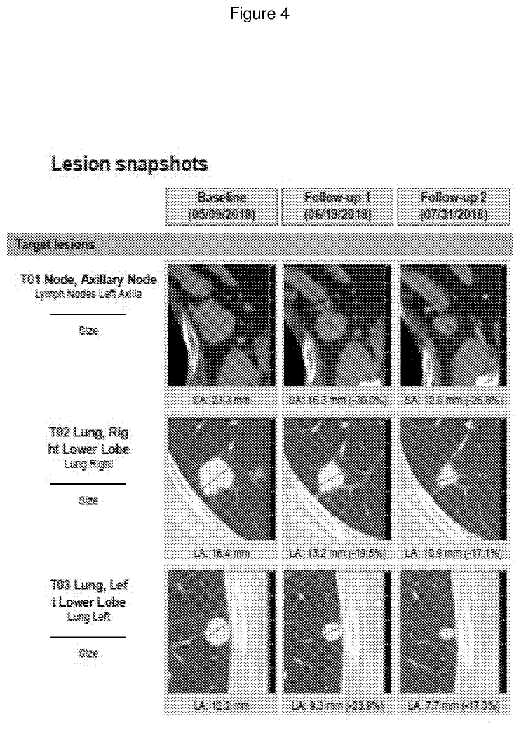

Lesion snapshots are provided in FIG. 4.

Subject 406

This 64 year old, white male patient was enrolled in the study GEN1021 and signed the informed consent form on the 11^{th} of June 2018 at a site in the US.

The patient was diagnosed with stage IV, non-small cell lung andenocarcinoma (negative for EGFR mutations and ALK rearrangement) on the 20^{th} of December 2016.

Past cancer treatments included carboplatin plus pemetrexed from December 2016 to February 2017, reported with progression during treatment and a best response of PD. The patient was treated with duruvalumab plus IPH-2201 (anti-NKG2A) from March 2017 to May 2017 with a best response of PD. The patient was subsequently treated with docetaxel plus ramucirumab from May 2017 to September 2017 with a best response of PD. Patient was treated with gemcitabine from October 2017 to January 2018, best response unknown but patient discontinued treatment due to PD. Patient received palliative radiotherapy in March 2018 (response to treatment not reported).

Medical history included hypertension, hyperlipidemia, fatigue, appetite and weight change, shortness of breath, depression and back pain. All conditions were ongoing at the time of enrollment.

Patient is a past-smoker (32 years) but discontinued smoking in January 2004. Patient was reported with an ECOG of 1 at the time of enrollment.

Patient received the first dose of enapotamab vendotin on C1D1 (20 Jun. 2018).

Treatment emerging events include two episodes of back pain (G2 and G3, both unrelated), neutropenia (G3, possibly related), fatigue (G2, not related), hypotension (G3, not related), hyponatremia (G3, not related), puritis (G1, possibly related), dry skin (G1, possibly related), neuropathy (G1, not related), anorexia (G2, not related), insomnia (G1, not related) and weight loss (G2, possibly related). Drug was interrupted due to G3 back pain but administration was not changed to any of the other events.

At screening two TLs were identified in the lung, a right lung lesion reported with a diameter of 18 mm and a left lung lesion reported with a diameter of 14 mm. The sum of diameters at screening was 32 mm. In addition, one NTL was identified, a bilateral lung lesion.

At C2D15 (8 Aug. 2018), first post-baseline scan was performed. At that time, the right lung lesion reported with a diameter of 8 mm and a left lung lesion reported with a diameter of 9 mm. The sum of diameters at C2D15 was 17 mm. As compared to screening, this corresponds to 46.8% decrease in sum of diameters. The NTL was reported as present (SD) and no new lesions were detected. The overall response assessment was reported as PR according to RECIST 1.1.

To date, patient is ongoing in Cycle 4 as of 18^{st} of September 2018 with a non-confirmed response of PR as per investigators assessment. Independent review of the tumor assessments has not been completed.

1. A conjugate of monomethyl auristatin or a functional analog or derivative thereof and an antibody or antigen-binding fragment thereof capable of binding to human Axl (SEQ ID NO: 1), comprising
a heavy chain variable (VH) region comprising the CDR1, CDR2, and CDR3 sequences of SEQ ID Nos.: 19, 20, and 21, respectively, and
a light chain variable (VL) region comprising the CDR1, CDR2, and CDR3 sequences of SEQ ID Nos.: 22, GAS, and 23, respectively;
for use in treating cancer in a subject, wherein
the cancer is non-small cell lung cancer (NSCLC), and
the conjugate is administered to the subject at a dose of about 1.8-about 2.6 mg/kg body weight once every three weeks or by weekly dosing of about 0.8-about 1.2 mg/kg body weight for three weeks, optionally followed by one treatment-free week.

2. The conjugate for use according to any one of the preceding claims, wherein the conjugate is administered to the subject at a dose of about 2.0-about 2.4 mg/kg body weight once every three weeks or by weekly dosing of about 0.6-about 1.4 mg/kg body weight for three weeks, optionally followed by one treatment-free week.

3. The conjugate for use according to any one of the preceding claims, wherein the conjugate is administered to the subject at a dose of about 2.2 mg/kg body weight once every three weeks or by weekly dosing of about 1.0 mg/kg body weight for three weeks, optionally followed by one treatment-free week.

4. The conjugate for use according to any one of the preceding claims, wherein the conjugate is administered to the subject by weekly dosing of about 0.4-1.0 mg/kg body weight.

5. The conjugate for use according to any one of the preceding claims, wherein the conjugate is administered to the subject by weekly dosing of about 0.6-1.0 mg/kg body weight.

6. The conjugate for use according to any one of the preceding claims, wherein the conjugate is administered to the subject by weekly dosing of about 0.4-0.8 mg/kg body weight.

7. The conjugate for use according to any one of the preceding claims, wherein the conjugate is administered to the subject by weekly dosing of about 0.5-0.7 mg/kg body weight.

8. The conjugate for use according to any one of the preceding claims, wherein the conjugate is administered to the subject by weekly dosing of about 0.6 mg/kg body weight.

9. The conjugate for use according to any one of the preceding claims, wherein the route of administration is intravenous.

10. The conjugate for use according to any one of the preceding claims, wherein treatment is continued at least until said subject has experienced progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the first dose of the conjugate.

11. The conjugate for use according to any one of the preceding claims, wherein treatment is continued until disease progression or unacceptable toxicity.

12. The conjugate for use according to any one of the preceding claims, wherein the non-small cell lung cancer is an adenocarcinoma.

13. The conjugate for use according to any one of the preceding claims, wherein the non-small cell lung cancer is characterized by, and/or the subject has, one or more sensitizing mutation(s) in the epidermal growth factor receptor (EGFR) amino acid sequence (SEQ ID NO: 3).

14. The conjugate for use according to any one of the preceding claims, wherein the sensitizing mutation in the epidermal growth factor receptor (EGFR) amino acid sequence is selected from the group consisting of:
i) An in-frame deletion and optionally insertion of one or more amino acids at position 746-751, such as any of the deletions and insertions defined in table 2,
ii) Substitution of a single amino acid at any one of positions 709, 715, 719, 720, 768, 858 and 861 such as any of the deletions and insertions defined in table 3, and
iii) An In-frame duplication and/or insertion selected from the duplications/insertions defined in Table 4;
amino acid numbering referring to the numbering of amino acids in SEQ ID NO: 3.

15. The conjugate for use according to any one of the preceding claims, wherein the non-small cell lung cancer is characterized by, and/or the subject has, at least one mutation in the EGFR amino acid sequence selected from L747S, D761Y, T790M, C797S, T854A, such as T790M, C797S, D761Y, and double mutations T790M/D761Y and T790/C797S; amino acid numbering referring to the numbering of amino acids in SEQ ID NO: 3.

16. The conjugate for use according to any one of the preceding claims, wherein the non-small cell lung cancer is characterized by, and/or the subject has, at least one mutation in the EGFR amino acid sequence, which induces or confers resistance of said subject to one or more EGFR tysrosine kinase inhibitors (EGFR-TKIs).

17. The conjugate for use according to any one of the preceding claims, wherein the EGFR-TKI is a first generation EGFR-TKI, a second generation EGFR-TKI or a third generation EGFR-TKI.

18. The conjugate for use according to any one of the preceding claims, wherein the one or more EGFR-TKIs is/are selected from the group consisting of erlotinib, osimertinib, gefintinib, olmutinib, nazartinib and avitinib.

19. The conjugate for use according to any one of the preceding claims, wherein the non-small cell lung cancer is not characterized by, and/and the subject does not have, a sensitizing epidermal growth factor receptor (EGFR) mutation.

20. The conjugate for use according to any one of the preceding claims, wherein the non-small cell lung cancer is characterized by expression of an epidermal growth factor receptor (EGFR) selected form the group consisting of:
i. a wild-type human EGFR; e.g. a human EFGR that comprises the sequence set forth in SEQ ID NO: 3 or a mature polypeptide thereof; and
ii. a human EGFR which is a variant of the EGFR in item i and which, when compared with the EGFR in item I, does not have any sensitizing mutations.

21. The conjugate for use according to any one of the preceding claims, wherein the non-small cell lung cancer is not characterized by, and/or the subject does not have, a sensitizing epidermal growth factor receptor (EGFR) mutation selected from the group consisting of:
i) An in-frame deletion and optionally insertion of one or more amino acids at position 746-751, such as any of the deletions and insertions defined in table 2,
ii) Substitution of a single amino acid at any one of positions 709, 715, 719, 720, 768, 858 and 861 such as any of the deletions and insertions defined in table 3, and
iii) An In-frame duplication and/or insertion selected from the duplications/insertions defined in Table 4;
amino acid numbering referring to the numbering of amino acids in SEQ ID NO: 3.

22. The conjugate for use according to any one of the preceding claims, wherein the non-small cell lung cancer is not characterized by having, and/or the subject does not have, a mutation in the EGFR amino acid sequence, which induces or confers resistance of said subject to one or more EGFR tysrosine kinase inhibitors (EGFR-TKIs), and/or the subject does not have such a mutation.

23. The conjugate for use according to any one of the preceding claims, wherein the non-small cell lung cancer is not characterized by, and/or the subject does not have a mutation in the EGFR amino acid sequence selected from L747S, D761Y, T790M, C797S, T854A, such as from T790M, C797S, D761Y, and double mutations T790M/D761Y and T790/C797S; amino acid numbering referring to the numbering of amino acids in SEQ ID NO: 3.

24. The conjugate for use according to any one of the preceding claims, wherein the non-small cell lung cancer is characterized by having a mutation in the gene coding for the ALK tyrosine kinase (ALK), which leads to rearrangement of the gene coding for ALK (SEQ ID NO: 4) with a gene coding for a fusion partner, to form a fusion oncogene.

25. The conjugate for use according to any one of the preceding claims, wherein the non-small cell lung cancer is characterized by, and/or the subject has, a mutation in the gene coding the ALK, said mutation leading to rearrangement of the gene coding for ALK with the gene (EML4) coding for Echinoderm microtubule-associated protein-like 4 (EMAPL4) (SEQ ID NO: 5) (and formation of an EML4-ALK fusion oncogene).

26. The conjugate for use according to any one of the preceding claims, wherein the non-small cell lung cancer is characterized by, and/or the subject has, a mutation in the gene coding for the ALK tyrosine kinase (ALK), leading to rearrangement of the gene coding for the ALK with a gene selected from the group consisting of
i. KIF5B coding for Kinesin-1 heavy chain (KINH) (SEQ ID NO: 6),
ii. KLC1 coding for Kinesin light chain 1 (KLC1) (SEQ ID NO: 7),
iii. TFG coding for Protein TFG (SEQ ID NO: 8),
iv. TPR coding for Nucleoprotein TPR(SEQ ID NO: 9),
v. HIP1 coding for Huntington-interacting protein 1 (HIP-1) (SEQ ID NO: 10),
vi. STRN coding for Striatin (SEQ ID NO: 11),
vii. DCTN1 coding for dynactin subunit 1 (SEQ ID NO: 12),
viii. SQSTM1 coding for sequestosome-1 (SEQ ID NO: 13),
ix. NPM1 coding for nucleophosmin (SEQ ID NO: 14),
x. BCL11A coding for B-cell lymphoma/leukemia 11A (SEQ ID NO: 15), and
xi. BIRC6 coding for baculoviral IAP repeat-containing protein (SEQ ID NO: 16);
and formation of the respective fusion oncogene selected from the group consisting of a KIF5B-ALK fusion oncogene, a KLC1-ALK fusion oncogene, a TFG-ALK fusion oncogene, a TPR-ALK fusion oncogene, an HIP1-ALK fusion oncogene, a STRN-ALK fusion oncogene, a DCTN1-ALK fusion oncogene, a SQSTM1-ALK fusion oncogene, a NPM1-ALK fusion oncogene, a BCL11A-ALK fusion oncogene and a BIRC6-ALK fusion oncogene.

27. The conjugate for use according to any one of the preceding claims, wherein the non-small cell lung cancer is characterized by expression of a wild-type human ALK tyrosine kinase; e.g. a human ALK tyrosine kinase that comprises the sequence set forth in SEQ ID NO: 4 or a mature polypeptide thereof.

28. The conjugate for use according to any one of the preceding claims, wherein the non-small cell lung cancer is characterized by not having a mutation in the gene coding for the ALK tyrosine kinase (ALK), leading to rearrangement of ALK with fusion partner to form a fusion oncogene and/or the subject does not have such a mutation.

29. The conjugate for use according to any one of the preceding claims, wherein the non-small cell lung cancer is characterized by not having a mutation in the gene coding for the ALK tyrosine kinase (ALK), leading to rearrangement of the gene (EML4) coding for Echinoderm microtubule-associated protein-like 4 (EMAPL4) (SEQ ID NO: 5) with ALK (SEQ ID NO: 4) and formation of an EML4-ALK fusion oncogene and/or the subject does not have such a mutation.

30. The conjugate for use according to any one of the preceding claims, wherein the non-small cell lung cancer is characterized by not having a mutation in any of the genes defined in claim 26.

31. The conjugate for use according to any one of the preceding claims, wherein the non-small cell lung cancer is not characterized by a mutation selected from the group consisting of
a sensitizing epidermal growth factor receptor (EGFR) mutation,
a mutation in the gene coding for the ALK tyrosine kinase (ALK), leading to rearrangement of EML4 with ALK and formation of an EML4-ALK fusion oncogene,
a mutation in the EGFR amino acid sequence, which induces or confers resistance of said subject to one or more EGFR tysrosine kinase inhibitors (EGFR-TKIs); and
the subject has been treated with a programmed cell death-1 (PD-1)/programmed cell death-1 (PD-1) inhibitor (e.g. nivolumab, genolimzumab, atezolizumab, durvalumab or avelumab) or with chemotherapy (e.g. chemotherapy comprising platinum, a taxane, pemetrexed and/or gemcitabine) and has failed with such previous treatment.

32. The conjugate for use according to any one of the preceding claims, wherein the non-small cell lung cancer is characterized by a mutation selected from the group consisting of
an sensitizing epidermal growth factor receptor (EGFR) mutation,
a mutation in the EGFR amino acid sequence, which induces or confers resistance of said subject to one or more EGFR tysrosine kinase inhibitors (EGFR-TKIs),
a mutation in the gene coding for the ALK tyrosine kinase (ALK), leading to rearrangement of EML4 with ALK and formation of an EML4-ALK fusion oncogene; and
the subject has been treated with an EGFR inhibitor (e.g. erlotinib, osimertinib, gefintinib, olmutinib, nazartinib and avitinib) or with a PD-1/PD-L1 inhibitor (e.g. nivolumab, genolimzumab, atezolizumab, durvalumab or avelumab) and has failed with such previous treatment.

33. The conjugate for use according to any one of the preceding claims, wherein the antibody comprises a VH region which is at least 90%, such as at least 95%, such as at least 97%, such as at least 99% identical to SEQ ID No: 17 and a VL region which is at least 90%, such as at least 95%, such as at least 97%, such as at least 99% identical to SEQ ID No: 18.

34. The conjugate for use according to any one of the preceding claims, wherein the antibody comprises a VH region comprising SEQ ID No: 17 and a VL region comprising SEQ ID No: 18.

35. The conjugate for use according to any one of the preceding claims, wherein the antibody is a monoclonal antibody or a monoclonal antigen-binding fragment thereof.

36. The conjugate for use according to any one of the preceding claims, wherein the antibody is a humanized or human antibody.

37. The conjugate for use according to any one of the preceding claims, wherein the antibody is an IgG1, such as human IgG1, optionally allotype IgG1m(f).

38. The conjugate for use according to any one of the preceding claims, wherein the antibody is enapotamab.

39. The conjugate for use according to any one of the preceding claims, further comprising a linker between the antibody or antigen-binding fragment and the monomethyl auristatin.

40. The conjugate for use according to any one of the preceding claims, wherein the linker is a cleavable linker.

41. The conjugate for use according to any one of the preceding claims, wherein MMAE is linked to the antibody with a linker, which is maleimidocaproyl-valine-citrulline-p-aminobenzyloxycarbonyl (mc-vc-PAB).

42. The conjugate for use according to any one of the preceding claims, wherein the linker has the formula -MC-vc-PAB-, wherein:
a) MC is: 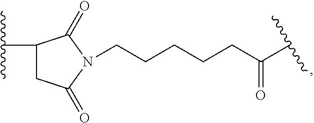 [SMILES: *C(=O)CCCCCN1C(=O)CC(*)C1=O]
b) vc is the dipeptide valine-citrulline, and
c) PAB is:

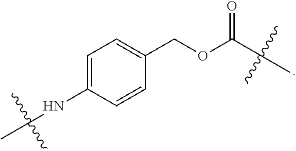 [SMILES: *Nc1ccc(COC(*)=O)cc1]

43. The conjugate for use according to claim 41 or 42, wherein the linker is attached to MMAE (vcMMAE), wherein vcMMAE is: 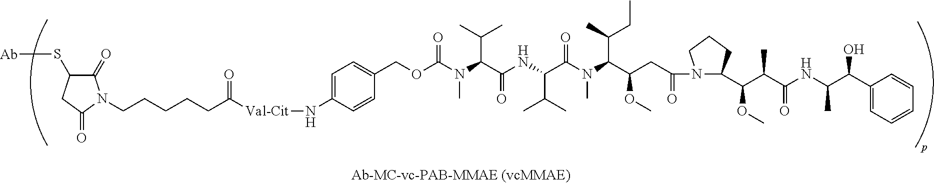
wherein p denotes a number from 1 to 8, S represents a sulphydryl residue of the antibody, and Ab designates the antibody or antigen-binding fragment thereof.

44. The conjugate for use according to any one of claims 41-43, wherein the average value of p in a population of the antibody-drug conjugate is about 4.

45. The conjugate for use according to any one of the preceding claims, wherein the conjugate is enapotamab vedotin.

46. A method of treating a cancer in a subject, the method comprising administering to the subject a conjugate of monomethyl auristatin or a functional analog or derivative thereof and an antibody or antigen-binding fragment thereof capable of binding to human Axl (SEQ ID NO: 1), comprising
a heavy chain variable (VH) region comprising the CDR1, CDR2, and CDR3 sequences of SEQ ID Nos.: 19, 20, and 21, respectively, and
a light chain variable (VL) region comprising the CDR1, CDR2, and CDR3 sequences of SEQ ID Nos.: 22, GAS, and 23, respectively; wherein
the cancer is non-small cell lung cancer (NSCLC), and
the conjugate is administered to the subject at a dose of about 1.8-about 2.6 mg/kg body weight once every three weeks or by weekly dosing of about 0.8-about 1.2 mg/kg body weight for three weeks, optionally followed by one treatment-free week.

47. The method according to claim 46, wherein the conjugate is administered to the subject at a dose of about 2.0-about 2.4 mg/kg body weight once every three weeks or by weekly dosing of about 0.6-about 1.4 mg/kg body weight for three weeks, optionally followed by one treatment-free week.

48. The method according to any one of claims 46-47, wherein the conjugate is administered to the subject at a dose of about 2.2 mg/kg body weight once every three weeks or by weekly dosing of about 1.0 mg/kg body weight for three weeks, optionally followed by one treatment-free week.

49. The method according to any one of claims 46-48, wherein the conjugate is administered to the subject by weekly dosing of about 0.4-1.0 mg/kg body weight.

50. The method according to any one of claims 46-48, wherein the conjugate is administered to the subject by weekly dosing of about 0.6-1.0 mg/kg body weight.

51. The method according to any one of claims 46-48, wherein the conjugate is administered to the subject by weekly dosing of about 0.4-0.8 mg/kg body weight.

52. The method according to any one of claims 46-48, wherein the conjugate is administered to the subject by weekly dosing of about 0.5-0.7 mg/kg body weight.

53. The method according to any one of claims 46-48, wherein the conjugate is administered to the subject by weekly dosing of about 0.6 mg/kg body weight.

54. The method according to any one of claims 46-53, wherein the route of administration is intravenous.

55. The method according to any one of claims 46-54, wherein treatment is continued at least until said subject has experienced progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the first dose of the conjugate.

56. The method according to any one of claims 46-55, wherein treatment is continued until disease progression or unacceptable toxicity.

57. The method according to any one of claims 46-56, wherein the non-small cell lung cancer is an adenocarcinoma.

58. The method according to any one of claims 46-57, wherein the non-small cell lung cancer is characterized by, and/or the subject has, one or more sensitizing mutation(s) in the epidermal growth factor receptor (EGFR) amino acid sequence (SEQ ID NO: 3).

59. The method according to any one of claims 46-58, wherein the sensitizing mutation in the epidermal growth factor receptor (EGFR) amino acid sequence is selected from the group consisting of:
i) An in-frame deletion and optionally insertion of one or more amino acids at position 746-751, such as any of the deletions and insertions defined in table 2,
ii) Substitution of a single amino acid at any one of positions 709, 715, 719, 720, 768, 858 and 861 such as any of the deletions and insertions defined in table 3, and
iii) An In-frame duplication and/or insertion selected from the duplications/insertions defined in Table 4;
amino acid numbering referring to the numbering of amino acids in SEQ ID NO: 3.

60. The method according to any one of claims 46-59, wherein the non-small cell lung cancer is characterized by, and/or the subject has, at least one mutation in the EGFR amino acid sequence selected from L747S, D761Y, T790M, C797S, T854A, such as T790M, C797S, D761Y, and double mutations T790M/D761Y and T790/C797S; amino acid numbering referring to the numbering of amino acids in SEQ ID NO: 3.

61. The method according to any one of claims 46-60, wherein the non-small cell lung cancer is characterized by, and/or the subject has, at least one mutation in the EGFR amino acid sequence, which induces or confers resistance of said subject to one or more EGFR tysrosine kinase inhibitors (EGFR-TKIs).

62. The method according to claim 61, wherein the EGFR-TKI is a first generation EGFR-TKI, a second generation EGFR-TKI or a third generation EGFR-TKI.

63. The method according to any one of claims 61-62, wherein the one or more EGFR-TKIs is/are selected from the group consisting of erlotinib, osimertinib, gefintinib, olmutinib, nazartinib and avitinib.

64. The method according to any one of claims 46-63, wherein the non-small cell lung cancer is not characterized by, and/and the subject does not have, a sensitizing epidermal growth factor receptor (EGFR) mutation.

65. The method according to any one of claims 46-64, wherein the non-small cell lung cancer is not characterized by, and/or the subject does not have, a sensitizing epidermal growth factor receptor (EGFR) mutation selected from the group consisting of:
i) An in-frame deletion and optionally insertion of one or more amino acids at position 746-751, such as any of the deletions and insertions defined in table 2,
ii) Substitution of a single amino acid at any one of positions 709, 715, 719, 720, 768, 858 and 861 such as any of the deletions and insertions defined in table 3, and
iii) An In-frame duplication and/or insertion selected from the duplications/insertions defined in Table 4;
amino acid numbering referring to the numbering of amino acids in SEQ ID NO: 3.

66. The method according to any one of claims 46-65, wherein the non-small cell lung cancer is not characterized by having, and/or the subject does not have, a mutation in the EGFR amino acid sequence, which induces or confers resistance of said subject to one or more EGFR tysrosine kinase inhibitors (EGFR-TKIs), and/or the subject does not have such a mutation.

67. The method according to any one of claims 46-66, wherein the non-small cell lung cancer is not characterized by, and/or the subject does not have a mutation in the EGFR amino acid sequence selected from L747S, D761Y, T790M, C797S, T854A, such as from T790M, C797S, D761Y, and double mutations T790M/D761Y and T790/C797S; amino acid numbering referring to the numbering of amino acids in SEQ ID NO: 3.

68. The method according to any one of claims 46-67, wherein the non-small cell lung cancer is characterized by having a mutation in the gene coding for the ALK tyrosine kinase (ALK), which leads to rearrangement of the gene coding for ALK (SEQ ID NO: 4) with a gene coding for a fusion partner, to form a fusion oncogene.

69. The method according to any one of claims 46-68, wherein the non-small cell lung cancer is characterized by, and/or the subject has, a mutation in the gene coding the ALK, said mutation leading to rearrangement of the gene coding for ALK with the gene (EML4) coding for Echinoderm microtubule-associated protein-like 4 (EMAPL4) (SEQ ID NO: 5) (and formation of an EML4-ALK fusion oncogene).

70. The method according to any one of claims 46-69, wherein the non-small cell lung cancer is characterized by, and/or the subject has, a mutation in the gene coding for the ALK tyrosine kinase (ALK), leading to rearrangement of the gene coding for the ALK with a gene selected from the group consisting of
i. KIF5B coding for Kinesin-1 heavy chain (KINH) (SEQ ID NO: 6),
ii. KLC1 coding for Kinesin light chain 1 (KLC1) (SEQ ID NO: 7),
iii. TFG coding for Protein TFG (SEQ ID NO: 8),
iv. TPR coding for Nucleoprotein TPR(SEQ ID NO: 9),
v. HIP1 coding for Huntington-interacting protein 1 (HIP-1) (SEQ ID NO: 10),
vi. STRN coding for Striatin (SEQ ID NO: 11),
vii. DCTN1 coding for dynactin subunit 1 (SEQ ID NO: 12),
viii. SQSTM1 coding for sequestosome-1 (SEQ ID NO: 13),
ix. NPM1 coding for nucleophosmin (SEQ ID NO: 14),
x. BCL11A coding for B-cell lymphoma/leukemia 11A (SEQ ID NO: 15), and
xi. BIRC6 coding for baculoviral IAP repeat-containing protein (SEQ ID NO: 16);
and formation of the respective fusion oncogene selected from the group consisting of a KIF5B-ALK fusion oncogene, a KLC1-ALK fusion oncogene, a TFG-ALK fusion oncogene, a TPR-ALK fusion oncogene, an HIP1-ALK fusion oncogene, a STRN-ALK fusion oncogene, a DCTN1-ALK fusion oncogene, a SQSTM1-ALK fusion oncogene, a NPM1-ALK fusion oncogene, a BCL11A-ALK fusion oncogene and a BIRC6-ALK fusion oncogene.

71. The method according to any one of claims 46-70, wherein the non-small cell lung cancer is characterized by not having a mutation in the gene coding for the ALK tyrosine kinase (ALK), leading to rearrangement of ALK with fusion partner to form a fusion oncogene and/or the subject does not have such a mutation.

72. The method according to any one of claims 46-71, wherein the non-small cell lung cancer is characterized by not having a mutation in the gene coding for the ALK tyrosine kinase (ALK), leading to rearrangement of the gene (EML4) coding for Echinoderm microtubule-associated protein-like 4 (EMAPL4) (SEQ ID NO: 5) with ALK (SEQ ID NO: 4) and formation of an EML4-ALK fusion oncogene and/or the subject does not have such a mutation.

73. The method according to any one of claims 46-72, wherein the non-small cell lung cancer is characterized by not having a mutation in any of the genes defined in claim 24.

74. The method according to any one of claims 46-73, wherein the non-small cell lung cancer is not characterized by a mutation selected from the group consisting of
an activating epidermal growth factor receptor (EGFR) mutation,
a mutation in the gene coding for the ALK tyrosine kinase (ALK), leading to rearrangement of EML4 with ALK and formation of an EML4-ALK fusion oncogene,
a mutation in the EGFR amino acid sequence, which induces or confers resistance of said subject to one or more EGFR tysrosine kinase inhibitors (EGFR-TKIs); and
the subject has been treated with a programmed cell death-1 (PD-1)/programmed cell death-1 (PD-1) inhibitor (e.g. nivolumab, genolimzumab, atezolizumab, durvalumab or avelumab) or with chemotherapy (e.g. chemotherapy comprising platinum, a taxane, pemetrexed and/or gemcitabine) and has failed with such previous treatment.

75. The method according to any one of claims 46-74, wherein the non-small cell lung cancer is characterized by a mutation selected from the group consisting of
an activating epidermal growth factor receptor (EGFR) mutation,
a mutation in the EGFR amino acid sequence, which induces or confers resistance of said subject to one or more EGFR tysrosine kinase inhibitors (EGFR-TKIs),
a mutation in the gene coding for the ALK tyrosine kinase (ALK), leading to rearrangement of EML4 with ALK and formation of an EML4-ALK fusion oncogene; and
the subject has been treated with an EGFR inhibitor (e.g. erlotinib, osimertinib, gefintinib, olmutinib, nazartinib and avitinib) or with a PD-1/PD-L1 inhibitor (e.g. nivolumab, genolimzumab, atezolizumab, durvalumab or avelumab) and has failed with such previous treatment.

76. The method according to any one of claims 46-75, wherein the antibody comprises a VH region which is at least 90%, such as at least 95%, such as at least 97%, such as at least 99% identical to SEQ ID No: 17 and a VL region which is at least 90%, such as at least 95%, such as at least 97%, such as at least 99% identical to SEQ ID No: 18.

77. The method according to any one of claims 46-76, wherein the antibody comprises a VH region comprising SEQ ID No: 17 and a VL region comprising SEQ ID No: 18.

78. The method according to any one of claims 46-77, wherein the antibody is a monoclonal antibody or a monoclonal antigen-binding fragment thereof.

79. The method according to any one of claims 46-78, wherein the antibody is a humanized or human antibody.

80. The method according to any one of claims 46-79, wherein the antibody is an IgG1, such as human IgG1, optionally allotype IgG1m(f).

81. The method according to any one of claims 46-80, wherein the antibody is enapotamab.

82. The method according to any one of claims 46-81 further comprising a linker between the antibody or antigen-binding fragment and the monomethyl auristatin.

83. The method according to any one of claims 46-82, wherein the linker is a cleavable linker.

84. The method according to any one of claims 46-83, wherein MMAE is linked to the antibody with a linker, which is maleimidocaproyl-valine-citrulline-p-aminobenzyloxycarbonyl (mc-vc-PAB).

85. The method according to any one of claims 46-84, wherein the linker has the formula -MC-vc-PAB-, wherein:
a) MC is: 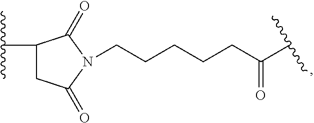 [SMILES: *C(=O)CCCCCN1C(=O)CC(*)C1=O]
b) vc is the dipeptide valine-citrulline, and
c) PAB is:

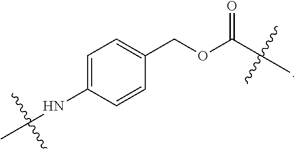 [SMILES: *Nc1ccc(COC(*)=O)cc1]

86. The method according to any one of claims 46-85, wherein the linker is attached to MMAE (vcMMAE), wherein vcMMAE is: 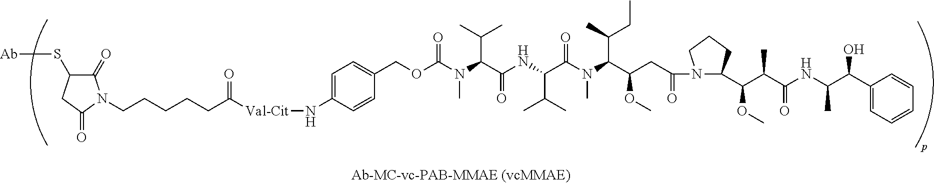
wherein p denotes a number from 1 to 8, S represents a sulphydryl residue of the antibody, and Ab designates the antibody or antigen-binding fragment thereof.

87. The method according to claim 86, wherein the average value of p in a population of the antibody-drug conjugate is about 4.

88. The method according to any one of claims 46-87, wherein the conjugate is enapotamab vedotin.

89. A kit comprising a conjugate of monomethyl auristatin or a functional analog or derivative thereof and an antibody or antigen-binding fragment thereof capable of binding to human Axl (SEQ ID NO: 1), comprising
a heavy chain variable (VH) region comprising the CDR1, CDR2, and CDR3 sequences of SEQ ID Nos.: 19, 20, and 21, respectively, and
a light chain variable (VL) region comprising the CDR1, CDR2, and CDR3 sequences of SEQ ID Nos.: 22, GAS, and 23, respectively;
and instructions for using the conjugate as set forth in any one of the preceding claims.
